(19) United States
(12) Patent Application Publication
(10) Pub. No.: US 2010/0317624 A1
(43) Pub. Date: Dec. 16, 2010

(54) HETEROCYCLIC UREA DERIVATIVES AND METHODS OF USE THEREOF

(75) Inventors: Allison Laura CHOY, Lexington, MA (US); Ann EAKIN, Stow, MA (US); Olga QUIROGA, Watertown, MA (US); Brian SHERER, Nashua, MA (US)

Correspondence Address: ASTRAZENECA R&D BOSTON, 35 GATEHOUSE DRIVE, WALTHAM, MA, 02451-1215, US

(73) Assignee: ASTRAZENECA AB

(21) Appl. No.: 12/794,894

(22) Filed: Jun. 7, 2010

Related U.S. Application Data
(60) Provisional application No. 61/184,914, filed on Jun. 8, 2009.
(60) Provisional application No. 61/285,010, filed on Dec. 9, 2009.

(51) Int. Cl. A61K 31/675; C07D 417/14; A61K 31/4709; C07F 9/6558; A61K 31/496; C07D 487/08; A61K 31/4995; A61K 31/5377; A61P 31/04

(52) U.S. Cl. 514/82; 546/156; 514/312; 546/23; 544/363; 514/253.08; 544/351; 514/249; 544/128; 514/236.8

(57) ABSTRACT

Compounds of formula (IA) and their pharmaceutically acceptable salts are described. Processes for their preparation, pharmaceutical compositions containing them, their use as medicaments and their use in the treatment of bacterial infections are also described.

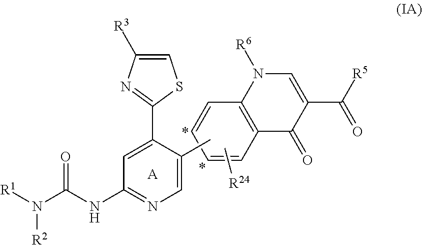

### FIELD OF THE INVENTION

The present invention relates to compounds that demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular, this invention relates to compounds useful for the treatment of bacterial infections in warm-blooded animals such as humans, more particularly to the use of these compounds in the manufacture of medicaments for use in the treatment of bacterial infections in warm-blooded animals such as humans.

### BACKGROUND OF THE INVENTION

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D.C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomerase IV are known in the art. For example, coumarin-containing compounds are described in patent application number WO 99/35155, 5,6-bicyclic heteroaromatic compounds are described in patent application WO 02/060879, and pyrazole compounds are described in patent application WO 01/52845 (US patent U.S. Pat. No. 6,608,087). AstraZeneca has also published certain applications describing anti-bacterial compounds: WO2005/026149, WO2006/087544, WO2006/087548, WO2006/087543, WO2006/092599, WO2006/092608, WO2007/071965. WO2008/020227, WO2008/020222, WO2008/020229, WO2008/068470, WO2008/152418, WO2009/027732, and WO2009/027733.

### SUMMARY OF THE INVENTION

We have discovered a new class of compounds which are useful for inhibiting DNA gyrase and/or topoisomerase IV.

In one embodiment, according to the present invention there is provided a compound of formula (IA):

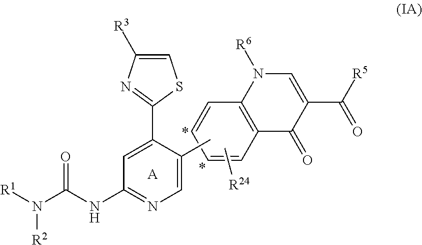

or a pharmaceutically acceptable salt thereof, wherein:

Ring A is attached to one of the carbon atoms indicated by “*”;

R¹ is selected from C₁₋₆alkyl, C₂₋₆alkenyl, C₂₋₆alkynyl or C₃₋₆cycloalkyl; wherein R¹ may be optionally substituted on carbon by one or more R⁷;

R² is selected from hydrogen or C₁₋₆alkyl; wherein said C₁₋₆alkyl may be optionally substituted by one or more groups independently selected from halo, cyano, hydroxy, nitro and amino;

or R¹ and R² together with the nitrogen to which they are attached form a heterocyclyl; wherein said heterocyclyl may be optionally substituted on one or more carbon atoms with one or more R⁸; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;

R³ is a C₁₋₆alkyl or a C₃₋₁₄carbocyclyl; wherein the alkyl or carbocyclyl may be optionally substituted on one or more carbon atoms by one or more R¹⁰;

R⁵ is —OH, a C₁₋₆alkoxy, an N—(C₁₋₆alkyl)amino, or N,N—(C₁₋₆alkyl)₂amino; wherein the C₁₋₆alkoxy, an N—(C₁₋₆alkyl)amino, or N,N—(C₁₋₆alkyl)₂-amino may be optionally substituted on one or more carbon atoms with one or more, independently selected R¹⁴;

R⁶ is selected from the group consisting of hydrogen, C₁₋₁₀alkyl, C₃₋₁₄-carbocyclyl-L-, and heterocycle-L-; wherein R⁶ is optionally substituted on one or more carbon atoms with one or more R¹⁶; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH-moiety that nitrogen may be optionally substituted by a group selected from R¹⁷; provided that one of R⁵ or R⁶ is substituted with phosphonooxy, or R²⁴ is not H;

L is a direct bond or a C₁₋₆alkylene;

R⁷, R⁸, and R¹⁰ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆carbocyclyl, and heterocyclyl; wherein R⁷, R⁸, and R¹⁰, independently of each other may be optionally substituted on one or more carbon by one or more R¹⁹; and wherein if said heterocyclyl contains an —NH— moiety that nitrogen may be optionally substituted by a group selected from R²⁰; and wherein if said heterocyclyl contains an ═N— or a —S— moiety that nitrogen may be optionally substituted by one oxo group and that sulfur may be optionally substituted by one or two oxo groups;

R¹⁴ and R¹⁶ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆-carbocyclyl, heterocyclyl and phosphonooxy; wherein R¹⁴ and R¹⁶ independently of each other may be optionally substituted on one or more carbon by one or more R²¹; and wherein if said heterocyclyl contains an —NH— moiety that nitrogen may be optionally substituted by a group selected from R²²; and wherein if said heterocyclyl contains an ═N— or a —S— moiety that nitrogen may be optionally substituted by one oxo group and that sulfur may be optionally substituted by one or two oxo groups;

R⁹, R¹⁷, R²⁰, and R²², for each occurrence, are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹⁷, R²⁰, and R²², independently of each other, may be optionally substituted on carbon by one or more R²³; and

R¹⁹, R²¹, and R²³, for each occurrence, are independently selected from halo, nitro, cyano, hydroxy, trifluoromethoxy, trifluoromethyl, amino, carboxy, carbamoyl, mercapto, sulphamoyl, C1-6alkyl, C1-6alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

R²⁴ is selected from the group consisting of hydrogen, halo, nitro, cyano, hydroxy, amino, mercapto, heterocyclyl, C₁₋₆alkyl, C₂₋₆alkenyl, C₂₋₆alkynyl, C₁₋₆alkoxy, N—(C₁₋₆alkyl)amino, N,N—(C₁₋₆alkyl)₂-amino, and C₁₋₆alkylsulfanyl; wherein R²⁴ may be optionally substituted on one or more carbon by one or more one or more R²⁵; wherein if said heterocyclyl contains an —NH— moiety that nitrogen may be optionally substituted by C₁₋₆alkyl;

R²⁵ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆carbocyclyl or heterocyclyl; wherein R²⁵ may be optionally substituted on one or more carbon by one or more R²⁶; and wherein if said heterocyclyl contains an —NH— moiety that nitrogen may be optionally substituted by a group selected from R²⁷; and wherein if said heterocyclyl contains an ═N— or a —S— moiety that nitrogen may be optionally substituted by one oxo group and that sulfur may be optionally substituted by one or two oxo groups;

R²⁶ and R²⁸, for each occurrence, are independently selected from halo, nitro, cyano, hydroxy, trifluoromethoxy, trifluoromethyl, amino, carboxy, carbamoyl, mercapto, sulphamoyl, C₁₋₆alkyl, C₁₋₆alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and

R²⁷, for each occurrence, is independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)₂-carbamoyl, benzyl, benzyloxycarbonyl, benzoyl and phenylsulphonyl; wherein R²⁷ may be optionally substituted on carbon by one or more R²⁸.

In another embodiment, according to the present invention there is provided a compound of formula (I):

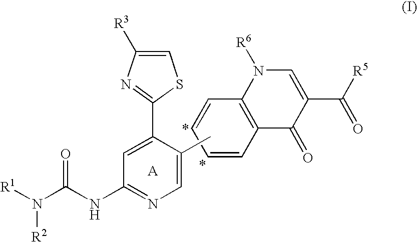

or a pharmaceutically acceptable salt thereof, wherein:

Ring A is attached to one of the carbon atoms indicated by “*”;

R¹ is selected from C₁₋₆alkyl, C₂₋₆alkenyl, C₂₋₆alkynyl or C₃₋₆cycloalkyl; wherein R¹ may be optionally substituted on carbon by one or more R⁷;

R² is selected from hydrogen or C₁₋₆alkyl; wherein said C₁₋₆alkyl may be optionally substituted by one or more groups independently selected from halo, cyano, hydroxy, nitro and amino;

or R¹ and R² together with the nitrogen to which they are attached form a heterocyclyl; wherein said heterocyclyl may be optionally substituted on one or more carbon atoms with one or more R⁸; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;

R³ is a C₁₋₆alkyl or a C₃₋₁₄carbocyclyl; wherein the alkyl or carbocyclyl may be optionally substituted on one or more carbon atoms by one or more R¹⁰;

R⁵ is —OH, a C₁₋₆alkoxy, an N—(C₁₋₆alkyl)amino, or N,N—(C₁₋₆alkyl)₂-amino; wherein the C₁₋₆alkoxy, an N—(C₁₋₆alkyl)amino, or N,N—(C₁₋₆alkyl)₂-amino may be optionally substituted on one or more carbon atoms with one or more, independently selected R¹⁴;

R⁶ is selected from the group consisting of hydrogen, C₁₋₁₀alkyl, C₃₋₁₄carbocyclyl-L-, and heterocycle-L-; wherein R⁶ is optionally substituted on one or more carbon atoms with one or more R¹⁶; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹⁷; provided that one of R⁵ or R⁶ is substituted with phosphonooxy;

L is a direct bond or a C₁₋₆alkylene;

R⁷, R⁸, and R¹⁰ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆carbocyclyl, and heterocyclyl; wherein R⁷, R⁸, and R¹⁰, independently of each other may be optionally substituted on one or more carbon by one or more R¹⁹; and wherein if said heterocyclyl contains an —NH— moiety that nitrogen may be optionally substituted by a group selected from R²⁰; and wherein if said heterocyclyl contains an ═N— or a —S— moiety that nitrogen may be optionally substituted by one oxo group and that sulfur may be optionally substituted by one or two oxo groups;

R¹⁴ and R¹⁶ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆carbocyclyl, heterocyclyl and phosphonooxy; wherein R¹⁴ and R¹⁶ independently of each other may be optionally substituted on one or more carbon by one or more R²¹; and wherein if said heterocyclyl contains an —NH— moiety that nitrogen may be optionally substituted by a group selected from R²²; and wherein if said heterocyclyl contains an ═N— or a —S— moiety that nitrogen may be optionally substituted by one oxo group and that sulfur may be optionally substituted by one or two oxo groups;

R⁹, R¹⁷, R²⁰, and R²², for each occurrence, are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹⁷, R²⁰, and R²², independently of each other, may be optionally substituted on carbon by one or more R²³; and

R¹⁹, R²¹, and R²³, for each occurrence, are independently selected from halo, nitro, cyano, hydroxy, trifluoromethoxy, trifluoromethyl, amino, carboxy, carbamoyl, mercapto, sulphamoyl, C1-6alkyl, C1-6alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl.

In another embodiment, the invention provides pharmaceutical compositions comprising a compound represented by formula (I) or (IA), or a pharmaceutically acceptable salt thereof, and a pharmaceutically acceptable excipient or carrier.

In another embodiment, the invention provides a method of inhibiting bacterial DNA gyrase and/or bacterial topoisomerase IV in a warm-blooded animal in need of such treatment, comprising administering to the animal an effective amount of a compound represented by formula (I) or (IA), or a pharmaceutically acceptable salt thereof. In a particular embodiment, the warm-blooded animal is a human.

In another embodiment, the invention provides a method of producing an antibacterial effect in a warm-blooded animal in need of such treatment, comprising administering to the animal an effective amount of a compound represented by formula (I) or (IA), or a pharmaceutically acceptable salt thereof. In a particular embodiment, the warm-blooded animal is a human.

In another embodiment, the invention provides a method of treating a bacterial infection in a warm-blooded animal in need thereof, comprising administering to the animal an effective amount of a compound represented by formula (I) or (IA), or a pharmaceutically acceptable salt thereof. In a particular embodiment, the warm-blooded animal is a human. In one embodiment, the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci. In a particular embodiment, the warm-blooded animal is a human.

In another embodiment, the invention provides the use of a compound represented by formula (I) or (IA), or a pharmaceutically acceptable salt thereof, for the manufacture of a medicament for use in the production of an antibacterial effect in a warm-blooded animal. In a particular embodiment, the warm-blooded animal is a human.

In another embodiment, the invention provides the use of a compound represented by formula (I) or (IA), or a pharmaceutically acceptable salt thereof, for the manufacture of a medicament for use in inhibition of bacterial DNA gyrase and/or topoisomerase IV in a warm-blooded animal. In a particular embodiment, the warm-blooded animal is a human.

In another embodiment, the invention provides the use of a compound represented by formula (I) or (IA), or a pharmaceutically acceptable salt thereof, for the manufacture of a medicament for use the treatment of a bacterial infection in a warm-blooded animal. In one embodiment, the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis and Vancomycin-Resistant Enterococci. In a particular embodiment, the warm-blooded animal is a human.

In another embodiment, the invention provides a compound represented by formula (I) or (IA), or a pharmaceutically acceptable salt thereof, for use in production of an anti-bacterial effect in a warm-blooded animal.

In another embodiment, the invention provides a compound represented by formula (I) or (IA), or a pharmaceutically acceptable salt thereof, for use in inhibition of bacterial DNA gyrase and/or topoisomerase IV in a warm-blooded animal.

In another embodiment, the invention provides a compound represented by formula (I) or (IA), or a pharmaceutically acceptable salt thereof, for use in the treatment of a bacterial infection in a warm-blooded animal.

In another embodiment, the invention provides a compound represented by formula (I) or (IA), or a pharmaceutically acceptable salt thereof, for use in the treatment of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis or Vancomycin-Resistant Enterococci.

### DETAILED DESCRIPTION OF THE INVENTION

In this specification the term alkyl includes both straight chained and branched saturated hydrocarbon groups. For example, “C₁₋₆alkyl” refers to an alkyl that has from 1 to 6 carbon atom and includes, for example, methyl, ethyl, propyl, isopropyl and t-butyl. However references to individual alkyl groups such as propyl are specific for the straight chain version only unless otherwise indicated (e.g., isopropyl). An analogous convention applies to other generic terms.

As used herein, the term “C₂₋₆akenyl” refers to a straight chained or branched hydrocarbon which has from 2 to 6 carbon atoms and has one or more double bond. Examples of “C₂₋₆alkenyl” are vinyl, allyl and 1-propenyl.

As used herein, the term “C₂₋₆akynyl” refers to a straight chained or branched hydrocarbon which has from 2 to 6 carbon atoms and has one or more triple bond. Examples of “C₂₋₆alkynyl” are ethynyl, 1-propynyl and 2-propynyl.

As used herein, the term “halo” refers to fluoro, chloro, bromo, and iodo.

A “heterocyclyl” is a saturated, partially saturated or unsaturated, mono or bicyclic ring containing 4-14 atoms of which at least one atom is chosen from nitrogen, sulphur or oxygen, which may, unless otherwise specified, be carbon or nitrogen linked, wherein a —CH₂-group can optionally be replaced by a —C(O)— and a ring nitrogen may be optionally substituted with one oxo to form an N-oxide and a ring sulfur may be optionally substituted with one or two oxo groups to form S-oxide(s). In one embodiment of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked. In a further aspect of the invention a “heterocyclyl” is an unsaturated, carbon-linked, monocyclic ring containing 5 or 6 atoms of which at least one atom is chosen from nitrogen, sulphur or oxygen. Examples and suitable values of the term “heterocyclyl” are morpholinyl, piperidyl, pyridinyl, pyranyl, pyrrolyl, pyrazolyl, isothiazolyl, indolyl, quinolinyl, thienyl, 1,3-benzodioxolyl, benzothiazolyl, thiadiazolyl, oxadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, 4,5-dihydro-oxazolyl, pyrimidinyl, pyrazinyl, pyridazinyl, isoxazolyl, thiazolyl, 1H-tetrazolyl, 1H-triazolyl, N-methylpyrrolyl, 4-pyridone, quinolin-4(1H)-one, pyridin-2(1H)-one, imidazo[1,2-a]pyridinyl, 1-isoquinolone, 2-pyrrolidone, 4-thiazolidone, quinoxalinyl, 5,6-dihydro[1,3]thiazolo[4,5-d]pyridazinyl, pyridine-N-oxide and quinoline-N-oxide. Suitable examples of “a nitrogen linked heterocyclyl” are morpholino, piperazin-1-yl, piperidin-1-yl and imidazol-1-yl. In a further aspect of the invention a “heterocyclyl” is a heteroaryl. The term “heteroaryl” refers to an unsaturated and aromatic heterocyclyl. Examples and suitable values for heteroaryl groups include pyridinyl, pyrrolyl, pyrazolyl, isothiazolyl, indolyl, quinolinyl, thienyl, benzothiazolyl, thiadiazolyl, oxadiazolyl, imidazolyl, pyrimidinyl, pyrazinyl, pyridazinyl, isoxazolyl, thiazolyl, 1H-tetrazolyl, 1H-triazolyl, N-methylpyrrolyl, quinolin-4(1H)-one, pyridin-2(1H)-one, imidazo[1,2-a]pyridinyl, 1-isoquinolone, quinoxalinyl, pyridine-N-oxide and quinoline-N-oxide. In a particular embodiment, the heteroaryl is a 5- or 6-membered heteroaryl, for example, pyridinyl, pyrrolyl, pyrazolyl, isothiazolyl, thienyl, thiadiazolyl, oxadiazolyl, imidazolyl, pyrimidinyl, pyrazinyl, pyridazinyl, isoxazolyl, thiazolyl, 1H-tetrazolyl, 1H-triazolyl, N-methylpyrrolyl, and pyridine-N-oxide.

A “carbocyclyl” is a saturated, partially saturated or unsaturated, mono-, bi- or tricyclic carbon ring that contains 3-14 atoms; wherein a —CH₂— group can optionally be replaced by a —C(O)—. In one embodiment, “carbocyclyl” is a monocyclic ring containing 5 or 6 atoms or a bicyclic ring containing 9 or 10 atoms. Examples of carbocyclyls include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. The term carbocyclyl encompasses both cycloalkyl and aryl groups. The term cycloalkyl refers to a carbocyclyl which is completely saturated, for example cyclopropyl, cyclobutyl, cyclopentyl, and cyclohexyl. The term “aryl” refers to a carbocyclyl which is completely unsaturated and is aromatic. A C₆₋₁₄aryl is an aromatic, mono-, bi- or tricyclic carbon ring that contains 6-14 atoms, for example phenyl or naphthenyl.

A “phosphonooxy” is a group having the following structure:

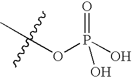

wherein

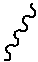

indicates the point of attachment to another moiety. A phosphonooxy group may lose one or two protons, for example when in solution depending on the pH of the solution, to form the following structures which are included in the definition of a phosphonooxy group:

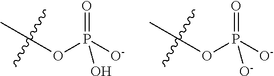

As used herein, a “C₁₋₆alkoxy” refers to a group which has a C₁₋₆alkyl that is attached to another moiety via an oxygen atom. Examples of “C₁₋₆alkoxy” are methoxy, ethoxy and propoxy.

As used herein, a “N—(C₁₋₆alkyl)amino” refers to a group which has a C₁₋₆alkyl that is attached to another moiety via —NH—. Examples of “N—(C₁₋₆alkyl)amino” are methylamino and ethylamino

As used herein, a “N,N—(C₁₋₆ alkyl)₂-amino” refers to a group which has two independently selected C₁₋₆alkyl that are attached to another moiety via a nitrogen atom. Examples of “N,N—(C₁₋₆alkyl)₂-amino” are N,N-dimethylamino, N,N-diethylamino and N-ethyl-N-methylamino

As used herein, a “C₁₋₆alkanoyloxy” refers to a group which has the formula —OC(O)R, wherein R is a C₁₋₆alkyl. An example of “C₁₋₆alkanoyloxy” is acetoxy.

As used herein, a “C₁₋₆alkoxycarbonyl” refers to a group which has the formula —C(O)OR, wherein R is a C₁₋₆alkyl. Examples of “C₁₋₆alkoxycarbonyl” are methoxycarbonyl, ethoxycarbonyl, n- and t-butoxycarbonyl.

As used herein, a “C₁₋₆alkoxycarbonylamino” refers to a group which has the formula —NHC(O)OR, wherein R is a C₁₋₆alkyl. Examples of “C₁₋₆alkoxycarbonylamino” are methoxycarbonylamino, ethoxycarbonylamino, n- and t-butoxycarbonylamino

As used herein, a “C₁₋₆alkanoylamino” refers to a group which has the formula —NHC(O)R, wherein R is a C₁₋₆alkyl. Examples of “C₁₋₆alkanoylamino” are formamido, acetamido and propionylamino

As used herein, a “C₁₋₆alkanoyl” refers to a group which has the formula —C(O)R, wherein R is a C₁₋₆alkyl. Examples of “C₁₋₆alkanoyl” are propionyl and acetyl.

As used herein, a “N—(C₁₋₆alkyl)sulphamoyl” refers to a group which has the formula —S(O)₂NHR, wherein R is a C₁₋₆alkyl. Examples of “N—(C₁₋₆alkyl)sulphamoyl” are N-(methyl)sulphamoyl and N-(ethyl)sulphamoyl.

As used herein, a “N,N—(C₁₋₆alkyl)₂sulphamoyl” refers to a group which has the formula —S(O)₂NR₂, wherein R, for each occurrence, is independently a C₁₋₆alkyl. Examples of “N,N—(C₁₋₆alkyl)₂sulphamoyl” are N,N-(dimethyl)sulphamoyl and N-(methyl)-N-(ethyl)sulphamoyl.

As used herein, a “N—(C₁₋₆alkyl)carbamoyl” refers to a group which has the formula —C(O)NHR, wherein R is a C₁₋₆alkyl. Examples of “N—(C₁₋₆alkyl)carbamoyl” are methylaminocarbonyl and ethylaminocarbonyl.

As used herein, a “N,N—(C₁₋₆alkyl)₂-carbamoyl” refers to a group which has the formula —C(O)NR₂, wherein R, for each occurrence, is independently a C₁₋₆alkyl. Examples of “N,N—(C₁₋₆alkyl)₂-carbamoyl” are dimethylaminocarbonyl and methylethylaminocarbonyl.

As used herein, a “C₁₋₆alkylsulphonylamino” refers to a group which has the formula —S(O)₂NHR, wherein R is a C₁₋₆alkyl. Examples of “C₁₋₆alkylsulphonylamino” are methylsulphonylamino, isopropylsulphonylamino and t-butylsulphonylamino

As used herein, a “C₁₋₆alkylsulphonyl” refers to a group which has the formula —S(O)₂R, wherein R is a C₁₋₆alkyl. Examples of “C₁₋₆alkylsulphonyl” are methylsulphonyl, isopropylsulphonyl and t-butylsulphonyl.

Examples of “C₁₋₆alkylS(O)ₐ wherein a is 0, 1, or 2” are methylthio, ethylthio, methylsulphinyl, ethylsulphinyl, mesyl and ethylsulphonyl.

The term “formula (I) or (IA)”, unless otherwise specified, refers to all embodiments of formula (I) or (IA) including but not limited to the specific examples disclosed herein.

A compound of formula (I) or (IA) may form stable acid or basic salts, and in such cases administration of a compound as a salt may be appropriate, and pharmaceutically acceptable salts may be made by conventional methods such as those described below.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula (I) or (IA), or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that certain compounds of formula (I) or (IA) contain an asymmetrically substituted carbon and/or sulphur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DNA gyrase and/or topoisomerase IV, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DNA gyrase and/or topoisomerase IV by the standard tests described hereinafter.

By way of clarity, compounds of the invention included all isotopes of the atoms present in formula (I) or (IA) and any of the examples or embodiments disclosed herein. For example, H (or hydrogen) represents any isotopic form of hydrogen including ¹H, ²H(D), and ³H(T); C represents any isotopic form of carbon including ¹²C, ¹³C, and ¹⁴C; O represents any isotopic form of oxygen including ¹⁶O, ¹⁷O and ¹⁸O; N represents any isotopic form of nitrogen including ¹³N, ¹⁴N and ¹⁵N; P represents any isotopic form of phosphorous including ³¹P and ³²P; S represents any isotopic form of sulfur including ³²S and ³⁵S; F represents any isotopic form of fluorine including ¹⁹F and ¹⁸F; Cl represents any isotopic form of chlorine including ³⁵Cl, ³⁷Cl and ³⁶Cl; and the like. In a preferred embodiment, compounds represented by formula (I) or (IA) comprises isomers of the atoms therein in their naturally occurring abundance. However, in certain instances, it is desirable to enrich one or more atom in a particular isotope which would normally be present in less abundance. For example, ¹H would normally be present in greater than 99.98% abundance; however, a compound of the invention can be enriched in ²H or ³H at one or more positions where H is present. In particular embodiments of the compounds of formula (I) or (IA), when, for example, hydrogen is enriched in the deuterium isotope, the symbol “D” is used to represent the enrichment in deuterium. In one embodiment, when a compound of the invention is enriched in a radioactive isotope, for example ³H and ¹⁴C, they may be useful in drug and/or substrate tissue distribution assays. It is to be understood that the invention encompasses all such isotopic forms which inhibit DNA gyrase and/or topoisomerase IV.

It is also to be understood that certain compounds of the formula (I) or (IA), and salts thereof can exist in solvated as well as unsolvated forms such as, for example, hydrated forms. It is to be understood that the invention encompasses all such solvated forms which inhibit DNA gyrase and/or topoisomerase IV.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

In one embodiment, the invention provides compounds of formula (IA), or a pharmaceutically acceptable salts thereof, wherein R²⁴ is selected from the group consisting of halo, heterocyclyl, C₁₋₆alkoxy, N—(C₁₋₆alkyl)amino; wherein R²⁴ may be optionally substituted on one or more carbon by one or more one or more R²⁵; wherein if said heterocyclyl contains an —NH— moiety that nitrogen may be optionally substituted by C₁₋₆alkyl. In particular embodiments, R²⁵ is N,N—(C₁₋₆alkyl)₂-amino. In specific embodiments, R²⁴ is fluoro, methoxy, 2-(dimethylamino)ethoxy, 2-(dimethylamino)ethyl, or 4-methylpiperazin-1-yl.

In certain embodiments, the invention provides compounds of formula (IA), or a pharmaceutically acceptable salts thereof, wherein the compounds are represented by the following formula (wherein the variables depicted are as described above for formula (IA))

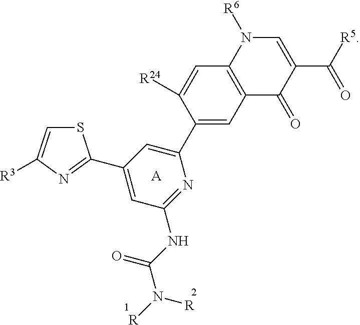

In one embodiment, the invention provides compounds of formula (I) or (IA), or pharmaceutically acceptable salts thereof, wherein R¹ is a C₁₋₆alkyl.

In another embodiment, the invention provides compounds of formula (I) or (IA), or pharmaceutically acceptable salts thereof, wherein R¹ is ethyl.

In another embodiment, the invention provides compounds of formula (I) or (IA), or pharmaceutically acceptable salts thereof, wherein R² is hydrogen.

In another embodiment, the invention provides compounds of formula (I) or (IA), or pharmaceutically acceptable salts thereof, wherein R³ is trifluouromethyl or cyclopropyl.

In another embodiment, the invention provides compounds of formula (I) or (IA), or pharmaceutically acceptable salts thereof, wherein R⁵ is selected from the group consisting of —OH, 2-phosphonooxy-ethylamino, and 3-phosphonooxy-propylamino

In another embodiment, the invention provides compounds of formula (I) or (IA), or pharmaceutically acceptable salts thereof, wherein R⁶ is a C₁₋₆alkyl which is substituted on one or more carbon atoms with one or more independently selected R¹⁶.

In another embodiment, the invention provides compounds of formula (I) or (IA), or pharmaceutically acceptable salts thereof, wherein R¹⁶, for each occurrence, is independently selected from hydroxy, a C₁₋₆alkoxy, a C₃₋₆carbocyclyl, a heterocyclyl, and phosphonooxy.

In another embodiment, the invention provides compounds of formula (I) or (IA), or pharmaceutically acceptable salts thereof, wherein R⁶ is ethyl, cyclopropylmethyl, isopentyl, propyl, 5-methyl-oxadiazol-3-ylmethyl, 1-phosphonooxy-4-methyl-pentan-2-yl, 1,3-dimethoxypropan-2-yl, 3,3-dimethylbutyl, 2-methoxyethyl, 1-phosphonooxy-butan-2-yl, 1-phosphonooxy-3,3-dimethyl-butan-2-yl, 1-phosphonooxy-3-methyl-butan-2-yl, or 1-methoxymethyl-2-methoxy-ethyl.

In another embodiment, the invention provides compounds of formula (I) or (IA), or pharmaceutically acceptable salts thereof, wherein R⁶ is ethyl, cyclopropylmethyl, isopentyl, propyl, 5-methyl-oxadiazol-3-ylmethyl, 1-phosphonooxy-4-methyl-pentan-2-yl, 1-phosphonooxy-butan-2-yl, 1-phosphonooxy-3,3-dimethyl-butan-2-yl, 1-phosphonooxy-3-methyl-butan-2-yl, or 1-methoxymethyl-2-methoxy-ethyl.

In another embodiment, the invention provides compounds of formula (I) or (IA), or pharmaceutically acceptable salts thereof, wherein R⁶ is a C₃₋₁₄carbocyclyl-L- or a heterocycle-L-, wherein the carbocyclyl or the heterocyclyl is substituted on one or more carbon atoms with one or more independently selected R¹⁶.

In another embodiment, the invention provides compounds of formula (I) or (IA), or pharmaceutically acceptable salts thereof, wherein R⁶ is cyclopropylmethyl, 1-ethylpyrrolidin-2-yl)methyl, (1-methyl-1H-imidazol-4-yl)methyl, 2-morpholinopropyl, (2-(diethylamino)ethyl)piperidin-3-yl, cyclohexyl, 5-methyl-oxadiazol-3-ylmethyl, or cyclopropyl.

In another embodiment, the invention provides compounds of formula (I) or (IA), or pharmaceutically acceptable salts thereof, wherein R⁶ is cyclopropylmethyl, 5-methyl-oxadiazol-3-ylmethyl, or cyclopropyl.

In another embodiment, the invention provides compounds of formula (IA), or a pharmaceutically acceptable salts thereof represented by the following formula:

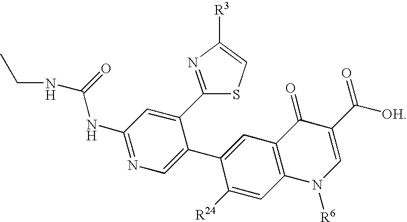

In another embodiment, the invention provides compounds of formula (IA), or a pharmaceutically acceptable salts thereof represented by the following formula:

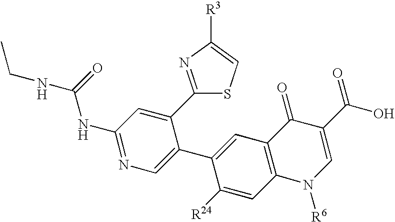

or a pharmaceutically acceptable salt thereof, wherein:

R³ is CF₃, or a phenyl that may be optionally substituted with halogen (e.g., F) or —OCH₃;

R⁶ is C₁₋₆alkyl, or a 4-6 membered heterocyclyl-L-; wherein the C₁₋₆alkyl is optionally substituted on one or more carbon atoms with hydroxy, phosphonooxy, N,N—(C₁₋₆alkyl)₂amino (e.g., dimethylamine or diethylamine) or C₁₋₆alkoxy; and wherein if said heterocyclyl contains an —NH— moiety that nitrogen may be optionally substituted by a group selected from C₁₋₃ alkyl, wherein the C₁₋₃alkyl may be optionally substituted with morpholinyl, or N-methyl-piperazinyl;

R²⁴ is selected from the group consisting of hydrogen, 5-6 membered heterocyclyl, halogen (e.g., F or Cl), or —O—(CH₂)₁₋₂—R₂₅; and wherein if said heterocyclyl contains an —NH— moiety that nitrogen may be optionally substituted by a group selected from C₁₋₃alkyl;

R²⁵ is selected from N—(C₁₋₆alkyl)amino or N,N—(C₁₋₆alkyl)₂-amino (e.g., dimethylamine or diethylamine);

L is a direct bond or a C₁₋₂alkyl. In a certain embodiments, R³ is CF₃ or a phenyl. In a particular embodiment, R³ is CF₃. In a certain embodiments, R²⁴ is N-methylpiperazinyl. In a certain embodiments, R²⁴ is 2-(diethylamino)ethoxy. In a certain embodiments, R²⁴ is 2-(dimethylamino)ethoxy. In a certain embodiments, R²⁴ is hydrogen. In a certain embodiments, R⁶ is ethyl. In a certain embodiments, R⁶ is (1-ethylpyrrolidin-2-yl)methyl. In a certain embodiments, R⁶ is 2-(dimethylamino)ethyl. In a certain embodiments, R⁶ is 1-phosphonooxy-4-methyl-pentan-2-yl.

In a particular embodiment, the present invention provides compounds having a structural formula (I) or (IA), or pharmaceutically acceptable salts thereof, as recited above wherein:

R¹ is a C₁₋₆alkyl;

R² is hydrogen;

R³ is a C₁₋₆alkyl or C₃₋₆carbocyclyl, wherein the alkyl or carbocycyl is optionally substituted with one or more halo;

R⁵ is —OH, a C₁₋₆alkoxy, or an N—(C₁₋₆alkyl)amino, wherein when R⁵ is an alkoxy or a N—(C₁₋₆alkyl)amino it is optionally substituted with phosphonooxy;

R⁶ is a C₁₋₆alkyl, a C₃₋₁₄carbocyclyl-L-, or a heterocyclyl-L-, wherein the alkyl, carbocyclyl-L and heterocyclyl are optionally substituted on one or more carbon atoms with one or more C₁₋₆alkly, C₁₋₆alkoxy, or phosphonooxy, provided that one of R⁵ or R⁶ is substituted with phosphonooxy.

In certain embodiments of formula (IA), one of R⁵ or R⁶ is substituted with phosphonooxy, and R²⁴ is not H.

In certain embodiments of formula (IA), one of R⁵ or R⁶ is not substituted with phosphonooxy, and R²⁴ is not H.

Particular compounds of the invention are the compounds of the Examples, and pharmaceutically acceptable salts thereof, each of which provides a further independent aspect of the invention.

In another embodiment, the invention provides pharmaceutical compositions comprising a pharmaceutically acceptable excipient or carrier and a compound represented by formula (I) or (IA), or a pharmaceutically acceptable salt thereof.

In a further aspect the present invention provides a process for preparing a compound of formula (I) or (IA), or a pharmaceutically-acceptable salt thereof, wherein variable groups in the schemes below are as defined in formula (I) or (IA) unless otherwise specified. In general, the compounds of the invention can be prepared by a palladium catalyzed Suzuki coupling reaction of a boronic ester or acid derivative (i) and a halo derivative (ii), as shown in Schemes I. Typically, the coupling reaction is heated and is carried out in the presence of a base such as Cs₂CO₃.

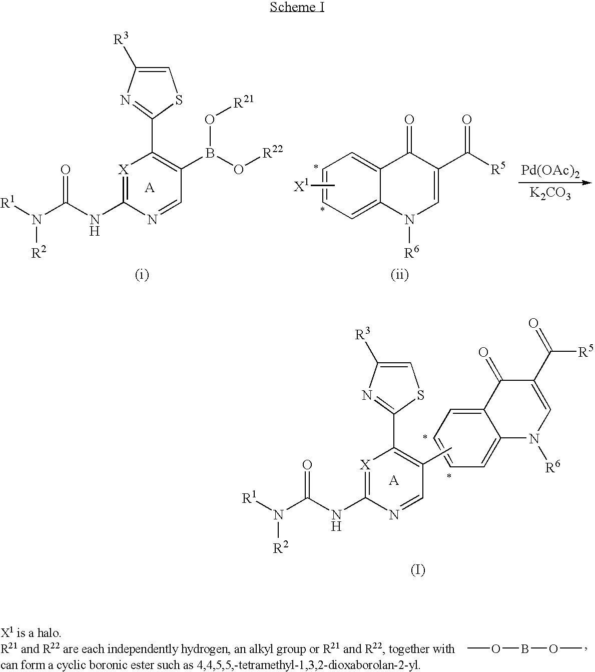

Boronic ester derivatives can be prepared by heating a halo derivative with a diboron compound such as 4,4,4′,4′,5,5,5′,5′-octamethyl-2,2′-bi(1,3,2-dioxaborolane) in the presence of 1,1′-bis(diphenylphosphino)ferrocene-palladium dichloride in an organic solvent.

R⁵ in Intermediate (ii) and (iv) is typically an alkoxy group which is generally stable during the Suzuki coupling reaction. R⁵ can be converted to an —OH group after the coupling reaction by treating the product with a strong base in a protic solvent such as water, an alcohol or a mixture of an alcohol and an organic solvent (e.g. THF/MeOH). R⁵ can be converted to an amino, an N-alkylamino or an N,N-dialkylamino by treating the ester derivative an ammonia, an N-alkylamine or an N,N-dialkylamine in an alcohol.

The urea portion of the compounds of the invention can be prepared from an isocyanate derivative either before or after the Suzuki coupling reaction from an amine derivative. If the Suzuki coupling reaction is preformed before formation of the urea, the amine is protected with an amine protecting group. When forming the urea derivative, the isocyanate derivative (iii) is typically combined with the amine derivative (iv) in an organic solvent and heated, as shown in Scheme II. The solvent can be aqueous, organic or a mixture of an aqueous miscible organic solvent and water.

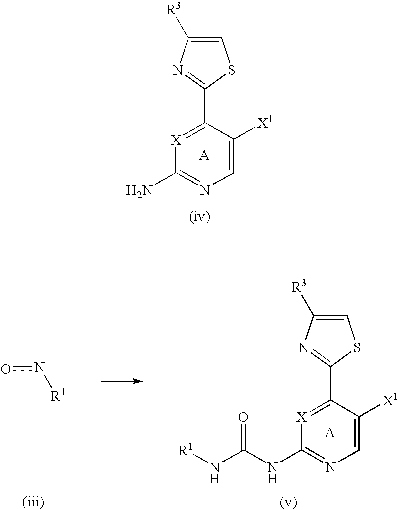

The thiazole ring can be added to ring A using a Suzuki coupling reaction as shown in Scheme III. Although Scheme III shows the coupling reaction of the thiazole ring occurring before the coupling reaction to link ring A to the 1,4-dihydroquinoline ring, the reactions could be preformed in the alternative order. When the thiazolyl group is attached before the coupling reaction to attach 1,4-dihydroquinoline ring, ring A can be brominated by heating it with 1-bromopyrrolidine-2,5-dione to form a substrate for the Suzuki coupling reaction shown in Scheme II.

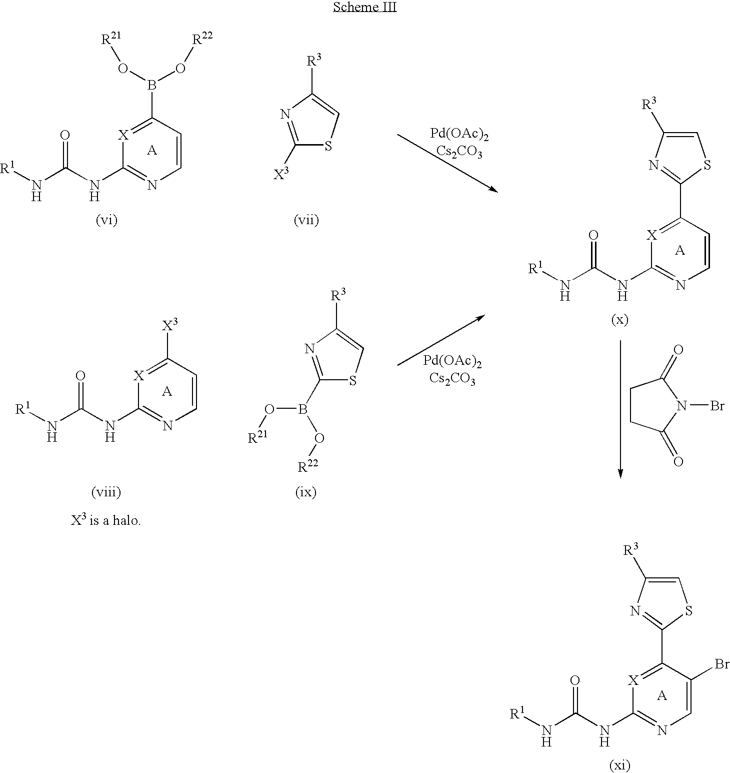

Alternatively, the thiazolyl group can be prepared from an ester derivative either before or after coupling of the 1,4-dihydroquinoline ring to ring A. For example, an ester derivative (xii) can be converted to an amide (xiii) by treating it with a solution of ammonia in an alcohol. The amide derivative (xiii) can then be converted to a thioamide (xiv) by treating the amide with Lawessons reagent. The thioamide (xiv) is then heated with an α-halo-ketone or an α-halo-aldehyde (xv) to form the thiazolyl group (v) (see Scheme IV). Although the thiazole ring is prepared before the Suzuki coupling reaction to attach 1,4-dihydroquinoline ring in Scheme IV, it could also be prepared after the coupling reaction of the ester derivative to the 1,4-dihydroquinoline ring.

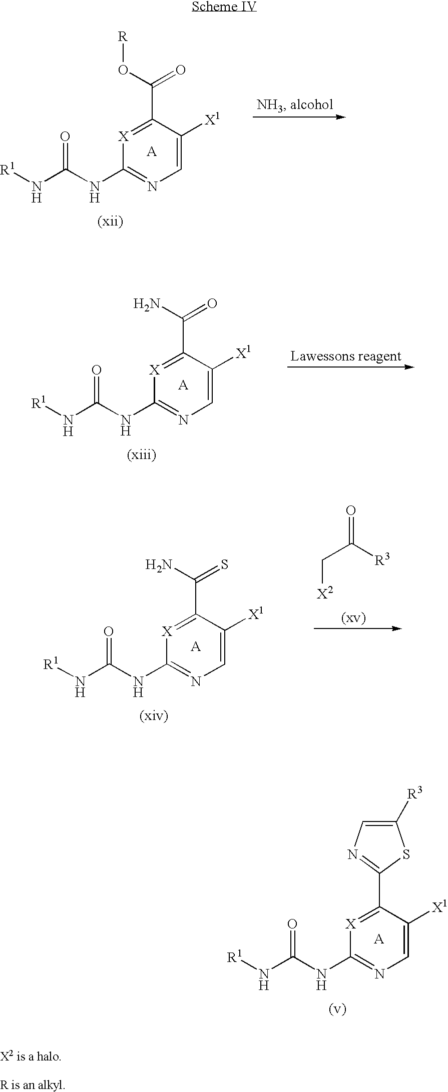

The 1,4-dihydroquinoline derivative (xvi) can be prepared from an alkyl (Z)-3-(dimethylamino)-2-(2-fluoro-5-halobenzoyl)acrylate or an alkyl (Z)-3-(dimethylamino)-2-(2-fluoro-4-halobenzoyl)acrylate (xvii) by heating it with a primary amine (xviii) in the presence of a base as shown in Scheme XII.

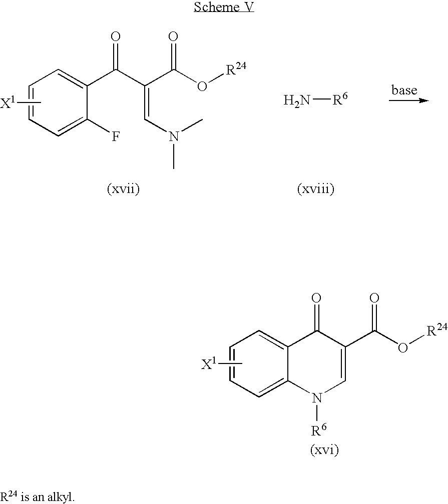

The phosphonooxy substituent on R⁵ or R⁶ can be prepared from a hydroxy substituent by treating the hydroxy derivative (for example (xix)) with a phosphoramidite (xx) in the presence of a weak acid such as tetrazole, followed by oxidation of the resulting alkyl phosphite derivative (xxi) with an oxidizing agent such as hydrogen peroxide to form an alkyl phosphate derivative (xxii) The alkyl groups can be removed by either treating the alkyl phosphate (xxii) with an acid such as HCl at room temperature or heating it with a base such as lithium hydroxide to form a phosphonooxy substituted compound of the invention (xxiii) (see Scheme VI).

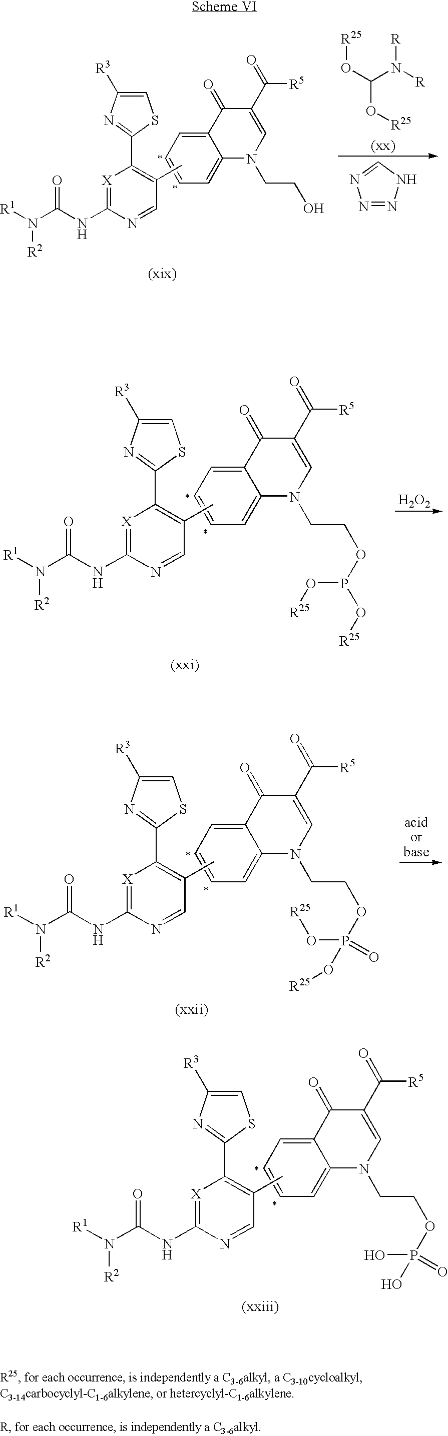

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) or (IA) into another compound of the formula (I) or (IA). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

An example of a suitable protecting group for a phosphonooxy group is an alkyl group such as t-butyl group which can be removed by treatment with an acid such as HCl. Other examples of protecting groups for a phosphonooxy group include an arylmethyl group such as a benzyl group which can be removed by treatment with trimethylsilyl bromide followed by water and/or an alcohol.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon, or for example, an allyl group which may be removed, for example, by use of a palladium catalyst such as palladium acetate.

The protecting groups may be removed at any convenient stage in the synthesis using conventional techniques well known in the chemical art, or they may be removed during a later reaction step or work-up.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

### Enzyme Potency Testing Methods

E. coli GyrB ATPase Inhibition Activity: Compounds can be tested for inhibition of E. coli GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays can be performed in multiwell plates in 30 μl reactions containing: 50 mM Hepes buffer pH 7.5, 75 mM ammonium acetate, 8.0 mM magnesium chloride, 0.5 mM ethylenediaminetetraacetic acid, 5% glycerol, 1 mM 1,4-Dithio-DL-threitol, 200 nM bovine serum albumin, 1.6 μg/ml sheared salmon sperm DNA, 400 pM E. coli GyrA, 400 pM E. coli GyrB, 250 μM ATP, and compound in dimethylsulfoxide. Reactions can be quenched with 30 μl of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values are calculated using dimethylsulfoxide (2%)-containing reactions as 0% inhibition and EDTA-containing (2.4 μM) reactions as 100% inhibition controls. An IC₅₀ measurement of compound potency for each compound can be determined from reactions performed in the presence of 10 different compound concentrations.

E. coli Topoisomerase IV ATPase Inhibition Activity: Compounds can be tested for inhibition of E. coli topoisomerase IV ATPase activity as described above for E. coli GyrB except the 30 μl reactions contained the following: 20 mM TRIS buffer pH 8, 50 mM ammonium acetate, 8 mM magnesium chloride, 5% glycerol, 5 mM 1,4-Dithio-DL-threitol, 0.005% Brij-35, 5 μg/ml sheared salmon sperm DNA, 500 pM E. coli ParC, 500 pM E. coli ParE, 160 μM ATP, and compound in dimethylsulfoxide. An IC₅₀ measurement of compound potency for each compound can be determined from reactions performed in the presence of 10 different compound concentrations.

Many of the compounds of the invention were tested in an assay substantially similar to the assays described above for measuring the inhibition of E. coli GyrB ATPase and E. coli Topoisomerase IV ATPase and had an IC₅₀ values of <200 μM in one or both assays.

S. aureus GyrB ATPase Inhibition Activity: Compounds may be tested for inhibition of S. aureus GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays can be performed in multiwell plates in 30 μl reactions containing: 50 mM Hepes buffer pH 7.5, 75 mM ammonium acetate, 8.0 mM magnesium chloride, 0.5 mM ethylenediaminetetraacetic acid, 5% glycerol, 1.0 mM 1,4-Dithio-DL-threitol, 200 nM bovine serum albumin, 1.0 μg/ml sheared salmon sperm DNA, 250 pM E. coli GyrA, 250 pM S. aureus GyrB, 250 μM ATP, and compound in dimethylsulfoxide. Reactions can be quenched with 30 μl of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values can be calculated using dimethylsulfoxide (2%)-containing reactions as 0% inhibition and EDTA-containing (2.4 μM) reactions as 100% inhibition controls. An IC₅₀ measurement of compound potency for each compound can be determined from reactions performed in the presence of 10 different compound concentrations.

The compounds in the table below were tested in an assay substantially similar to the assay described above for measuring the inhibition of S. aureus GyrB ATPase and were found to have a percent inhibition of S. aureus GyrB ATPase as shown in the table.

| | Percent Inhibition |
|---|---|
| Example | (1 μM) |
| 1 | No data |
| 2 | No data |
| 3 | 108 |
| 4 | No data |
| 5 | 120 |
| 6 | 96 |
| 7 | 111 |
| 8 | 108 |
| 9 | 103 |
| 10 | 12 |
| 11 | 108 |
| 12 | 104 |
| 13 | 99 |
| 14 | 97 |
| 15 | 76 |
| 16 | 100 |
| 17 | 100 |
| 18 | 104 |
| 19 | 110 |
| 20 | 106 |
| 21 | No data |
| 22 | 103 |
| 23 | 100 |
| 24 | 107 |
| 25 | 98 |
| 26 | 108 |
| 27 | 125 |
| 28 | 104 |
| 29 | 93 |
| 30 | 98 |
| 31 | 114 |
| 32 | 104 |
| 33 | 104 |
| 34 | 112 |
| 35 | 108 |
| 36 | 112 |
| 37 | 100 |
| 38 | 105 |
| 39 | 102 |
| 40 | 97 |
| 41 | 107 |
| 42 | 104 |
| 43 | 103 |
| 44 | 98 |
| 45 | 102 |
| 46 | 108 |
| 47 | 110 |
| 48 | 99 |
| 49 | 105 |
| 50 | 106 |
| 51 | 95 |
| 52 | 103 |
| 53 | 46 |
| 54 | 126 |
| 55 | 100 |
| 56 | 101 |

According to a further feature of the invention there is provided a compound of the formula (I) or (IA), or a pharmaceutically-acceptable salt thereof, for use in a method of treatment of the human or animal body by therapy.

In one embodiment, the invention provides a method of treating a bacterial infection in an animal, such as a human, comprising administering to the animal or human an effective amount of a compound of any one of formulas (I) or (IA), or a pharmaceutically acceptable salt thereof.

We have found that compounds of the present invention inhibit bacterial DNA gyrase and/or topoisomerase IV and are therefore of interest for their antibacterial effects. In one aspect of the invention the compounds of the invention inhibit bacterial DNA gyrase and are therefore of interest for their antibacterial effects. In one aspect of the invention, the compounds of the invention inhibit topoisomerase IV and are therefore of interest for their antibacterial effects. In one aspect of the invention, the compounds of the invention inhibit both DNA gyrase and topoisomerase IV and are therefore of interest for their antibacterial effects. Thus, the compounds of the invention are useful in treating or preventing bacterial infections.

In one aspect of the invention an “infection” or “bacterial infection” refers to an infection caused by Acinetobacter baumanii. In one aspect of the invention an “infection” or “bacterial infection” refers to an infection caused by Acinetobacter haemolyticus. In one aspect of the invention an “infection” or “bacterial infection” refers to an infection caused by Acinetobacter junii. In one aspect of the invention an “infection” or “bacterial infection” refers to an infection caused by Acinetobacter johnsonii. In one aspect of the invention an “infection” or “bacterial infection” refers to an infection caused by Acinetobacter lwoffi. In one aspect of the invention an “infection” or “bacterial infection” refers to an infection caused by Bacteroides bivius. In one aspect of the invention an “infection” or “bacterial infection” refers to an infection caused by Bacteroides fragilis. In one aspect of the invention an “infection” or “bacterial infection” refers to an infection caused by Burkholderia cepacia. In one aspect of the invention an “infection” or “bacterial infection” refers to an infection caused by Campylobacter jejuni. In one aspect of the invention an “infection” or “bacterial infection” refers to an infection caused by Chlamydia pneumoniae. In one aspect of the invention an “infection” or “bacterial infection” refers to an infection caused by Chlamydia urealyticus. In one aspect of the invention an “infection” or “bacterial infection” refers to an infection caused by Chlamydophila pneumoniae. In one aspect of the invention an “infection” or “bacterial infection” refers to an infection caused by Clostridium difficile. In one aspect of the invention an “infection” or “bacterial infection” refers to an infection caused by Enterobacter aerogenes. In one aspect of the invention an “infection” or “bacterial infection” refers to an infection caused by Enterobacter cloacae. In one aspect of the invention an “infection” or “bacterial infection” refers to an infection caused by Enterococcus faecalis. In one aspect of the invention an “infection” or “bacterial infection” refers to an infection caused by Enterococcus faecium. In one aspect of the invention an “infection” or “bacterial infection” refers to an infection caused by Escherichia coli. In one aspect of the invention an “infection” or “bacterial infection” refers to an infection caused by Gardnerella vaginalis. In one aspect of the invention an “infection” or “bacterial infection” refers to an infection caused by Haemophilus parainfluenzae. In one aspect of the invention an “infection” or “bacterial infection” refers to an infection caused by Haemophilus influenzae. In one aspect of the invention an “infection” or “bacterial infection” refers to an infection caused by Helicobacter pylori. In one aspect of the invention an “infection” or “bacterial infection” refers to an infection caused by Klebsiella pneumoniae. In one aspect of the invention an “infection” or “bacterial infection” refers to an infection caused by Legionella pneumophila. In one aspect of the invention an “infection” or “bacterial infection” refers to an infection caused by Methicillin-resistant Staphylococcus aureus. In one aspect of the invention an “infection” or “bacterial infection” refers to an infection caused by Methicillin-susceptible Staphylococcus aureus. In one aspect of the invention an “infection” or “bacterial infection” refers to an infection caused by Moraxella catarrhalis. In one aspect of the invention an “infection” or “bacterial infection” refers to an infection caused by Morganella morganii. In one aspect of the invention an “infection” or “bacterial infection” refers to an infection caused by Mycoplasma pneumoniae. In one aspect of the invention an “infection” or “bacterial infection” refers to an infection caused by Neisseria gonorrhoeae. In one aspect of the invention an “infection” or “bacterial infection” refers to an infection caused by Penicillin-resistant Streptococcus pneumoniae. In one aspect of the invention an “infection” or “bacterial infection” refers to an infection caused by Penicillin-susceptible Streptococcus pneumoniae. In one aspect of the invention an “infection” or “bacterial infection” refers to an infection caused by Peptostreptococcus magnus. In one aspect of the invention an “infection” or “bacterial infection” refers to an infection caused by Peptostreptococcus micros. In one aspect of the invention an “infection” or “bacterial infection” refers to an infection caused by Peptostreptococcus anaerobius. In one aspect of the invention an “infection” or “bacterial infection” refers to an infection caused by Peptostreptococcus asaccharolyticus. In one aspect of the invention an “infection” or “bacterial infection” refers to an infection caused by Peptostreptococcus prevotii. In one aspect of the invention an “infection” or “bacterial infection” refers to an infection caused by Peptostreptococcus tetradius. In one aspect of the invention an “infection” or “bacterial infection” refers to an infection caused by Peptostreptococcus vaginalis. In one aspect of the invention an “infection” or “bacterial infection” refers to an infection caused by Proteus mirabilis. In one aspect of the invention an “infection” or “bacterial infection” refers to an infection caused by Pseudomonas aeruginosa. In one aspect of the invention an “infection” or “bacterial infection” refers to an infection caused by Quinolone-Resistant Staphylococcus aureus. In one aspect of the invention an “infection” or “bacterial infection” refers to an infection caused by Quinolone-Resistant Staphylococcus epidermis. In one aspect of the invention an “infection” or “bacterial infection” refers to an infection caused by Salmonella typhi. In one aspect of the invention an “infection” or “bacterial infection” refers to an infection caused by Salmonella paratyphi. In one aspect of the invention an “infection” or “bacterial infection” refers to an infection caused by Salmonella enteritidis. In one aspect of the invention an “infection” or “bacterial infection” refers to an infection caused by Salmonella typhimurium. In one aspect of the invention an “infection” or “bacterial infection” refers to an infection caused by Serratia marcescens. In one aspect of the invention an “infection” or “bacterial infection” refers to an infection caused by Staphylococcus aureus. In one aspect of the invention an “infection” or “bacterial infection” refers to an infection caused by Staphylococcus epidermidis. In one aspect of the invention an “infection” or “bacterial infection” refers to an infection caused by Staphylococcus saprophyticus. In one aspect of the invention an “infection” or “bacterial infection” refers to an infection caused by Streptoccocus agalactiae. In one aspect of the invention an “infection” or “bacterial infection” refers to an infection caused by Streptococcus pneumoniae. In one aspect of the invention an “infection” or “bacterial infection” refers to an infection caused by Streptococcus pyogenes. In one aspect of the invention an “infection” or “bacterial infection” refers to an infection caused by Stenotrophomonas maltophilia. In one aspect of the invention an “infection” or “bacterial infection” refers to an infection caused by Ureaplasma urealyticum. In one aspect of the invention an “infection” or “bacterial infection” refers to an infection caused by Vancomycin-Resistant Enterococcus faecium. In one aspect of the invention an “infection” or “bacterial infection” refers to an infection caused by Vancomycin-Resistant Enterococcus faecalis. In one aspect of the invention an “infection” or “bacterial infection” refers to an infection caused by Vancomycin-Resistant Staphylococcus aureus. In one aspect of the invention an “infection” or “bacterial infection” refers to an infection caused by Vancomycin-Resistant Staphylococcus epidermis. In one aspect of the invention an “infection” or “bacterial infection” refers to an infection caused by Mycobacterium tuberculosis. In one aspect of the invention an “infection” or “bacterial infection” refers to an infection caused by Clostridium perfringens. In one aspect of the invention an “infection” or “bacterial infection” refers to an infection caused by Klebsiella oxytoca. In one aspect of the invention an “infection” or “bacterial infection” refers to an infection caused by Neisseria miningitidis. In one aspect of the invention an “infection” or “bacterial infection” refers to an infection caused by Fusobacterium spp. In one aspect of the invention an “infection” or “bacterial infection” refers to an infection caused by Peptococcus spp. In one aspect of the invention an “infection” or “bacterial infection” refers to an infection caused by Proteus vulgaris. In one aspect of the invention an “infection” or “bacterial infection” refers to an infection caused by Coagulase-negative Staphylococcus (including Staphylococcus lugdunensis, Staphylococcus capitis, Staphylococcus hominis, and Staphylococcus saprophyticus).

In one aspect of the invention an “infection” or “bacterial infection” refers to an infection caused by Acinetobacter spp. In one aspect of the invention an “infection” or “bacterial infection” refers to an infection caused by Bacteroides spp. In one aspect of the invention an “infection” or “bacterial infection” refers to an infection caused by Burkholderia spp. In one aspect of the invention an “infection” or “bacterial infection” refers to an infection caused by Campylobacter spp. In one aspect of the invention an “infection” or “bacterial infection” refers to an infection caused by Chlamydia spp. In one aspect of the invention an “infection” or “bacterial infection” refers to an infection caused by Chlamydophila spp. In one aspect of the invention an “infection” or “bacterial infection” refers to an infection caused by Clostridium spp. In one aspect of the invention an “infection” or “bacterial infection” refers to an infection caused by Enterobacter spp. In one aspect of the invention an “infection” or “bacterial infection” refers to an infection caused by Enterococcus spp. In one aspect of the invention an “infection” or “bacterial infection” refers to an infection caused by Escherichia spp. In one aspect of the invention an “infection” or “bacterial infection” refers to an infection caused by Gardnerella spp. In one aspect of the invention an “infection” or “bacterial infection” refers to an infection caused by Haemophilus spp. In one aspect of the invention an “infection” or “bacterial infection” refers to an infection caused by Helicobacter spp. In one aspect of the invention an “infection” or “bacterial infection” refers to an infection caused by Klebsiella spp. In one aspect of the invention an “infection” or “bacterial infection” refers to an infection caused by Legionella spp. In one aspect of the invention an “infection” or “bacterial infection” refers to an infection caused by Moraxella spp. In one aspect of the invention an “infection” or “bacterial infection” refers to an infection caused by Morganella spp. In one aspect of the invention an “infection” or “bacterial infection” refers to an infection caused by Mycoplasma spp. In one aspect of the invention an “infection” or “bacterial infection” refers to an infection caused by Neisseria spp. In one aspect of the invention an “infection” or “bacterial infection” refers to an infection caused by Peptostreptococcus spp. In one aspect of the invention an “infection” or “bacterial infection” refers to an infection caused by Proteus spp. In one aspect of the invention an “infection” or “bacterial infection” refers to an infection caused by Pseudomonas spp. In one aspect of the invention an “infection” or “bacterial infection” refers to an infection caused by Salmonella spp. In one aspect of the invention an “infection” or “bacterial infection” refers to an infection caused by Serratia spp. In one aspect of the invention an “infection” or “bacterial infection” refers to an infection caused by Staphylococcus spp. In one aspect of the invention an “infection” or “bacterial infection” refers to an infection caused by Streptoccocus spp. In one aspect of the invention an “infection” or “bacterial infection” refers to an infection caused by Stenotrophomonas spp. In one aspect of the invention an “infection” or “bacterial infection” refers to an infection caused by Ureaplasma spp. In one aspect of the invention an “infection” or “bacterial infection” refers to an infection caused by aerobes. In one aspect of the invention an “infection” or “bacterial infection” refers to an infection caused by obligate anaerobes. In one aspect of the invention an “infection” or “bacterial infection” refers to an infection caused by facultative anaerobes. In one aspect of the invention an “infection” or “bacterial infection” refers to an infection caused by gram-positive bacteria. In one aspect of the invention an “infection” or “bacterial infection” refers to an infection caused by gram-negative bacteria. In one aspect of the invention an “infection” or “bacterial infection” refers to an infection caused by gram-variable bacteria. In one aspect of the invention an “infection” or “bacterial infection” refers to an infection caused by atypical respiratory pathogens. In one aspect of the invention an “infection” or “bacterial infection” refers to an infection caused by Enterics. In one aspect of the invention an “infection” or “bacterial infection” refers to an infection caused by Shigella spp. In one aspect of the invention an “infection” or “bacterial infection” refers to an infection caused by Citrobacter.

In one aspect of the invention “infection” or “bacterial infection” refers to a gynecological infection. In one aspect of the invention “infection” or “bacterial infection” refers to a respiratory tract infection (RTI). In one aspect of the invention “infection” or “bacterial infection” refers to a sexually transmitted disease. In one aspect of the invention “infection” or “bacterial infection” refers to a urinary tract infection. In one aspect of the invention “infection” or “bacterial infection” refers to acute exacerbation of chronic bronchitis (ACEB). In one aspect of the invention “infection” or “bacterial infection” refers to acute otitis media. In one aspect of the invention “infection” or “bacterial infection” refers to acute sinusitis. In one aspect of the invention “infection” or “bacterial infection” refers to an infection caused by drug resistant bacteria. In one aspect of the invention “infection” or “bacterial infection” refers to catheter-related sepsis. In one aspect of the invention “infection” or “bacterial infection” refers to chancroid. In one aspect of the invention “infection” or “bacterial infection” refers to chlamydia. In one aspect of the invention “infection” or “bacterial infection” refers to community-acquired pneumonia (CAP). In one aspect of the invention “infection” or “bacterial infection” refers to complicated skin and skin structure infection. In one aspect of the invention “infection” or “bacterial infection” refers to uncomplicated skin and skin structure infection. In one aspect of the invention “infection” or “bacterial infection” refers to endocarditis. In one aspect of the invention “infection” or “bacterial infection” refers to febrile neutropenia. In one aspect of the invention “infection” or “bacterial infection” refers to gonococcal cervicitis. In one aspect of the invention “infection” or “bacterial infection” refers to gonococcal urethritis. In one aspect of the invention “infection” or “bacterial infection” refers to hospital-acquired pneumonia (HAP). In one aspect of the invention “infection” or “bacterial infection” refers to osteomyelitis. In one aspect of the invention “infection” or “bacterial infection” refers to sepsis. In one aspect of the invention “infection” or “bacterial infection” refers to syphilis. In one aspect of the invention “infection” or “bacterial infection” refers to ventilator-associated pneumonia. In one aspect of the invention “infection” or “bacterial infection” refers to intraabdominal infections. In one aspect of the invention “infection” or “bacterial infection” refers to gonorrhoeae. In one aspect of the invention “infection” or “bacterial infection” refers to meningitis. In one aspect of the invention “infection” or “bacterial infection” refers to tetanus. In one aspect of the invention “infection” or “bacterial infection” refers to tuberculosis.

In one embodiment, it is expected that the compounds of the present invention will be useful in treating bacterial infections including, but not limited to community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I) or (IA), or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I) or (IA), or a pharmaceutically acceptable salt thereof as defined hereinbefore.

According to a further feature of the invention there is provided a method of treating a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococciin a warm-blooded animal, such as a human being, in need of such treatment which comprises administering to said animal an effective amount of a compound of formula (I) or (IA), or a pharmaceutically acceptable salt thereof as defined hereinbefore.

A further feature of the present invention is a compound of formula (I) or (IA), and pharmaceutically acceptable salts thereof for use as a medicament. Suitably the medicament is an antibacterial agent.

According to a further aspect of the invention there is provided the use of a compound of formula (I) or (IA), or a pharmaceutically acceptable salt thereof in the manufacture of a medicament for use in the production of an anti-bacterial effect in a warm-blooded animal such as a human being.

According to a further aspect of the invention there is provided the use of a compound of formula (I) or (IA), or a pharmaceutically acceptable salt thereof, in the manufacture of a medicament for use in inhibition of bacterial DNA gyrase and/or topoisomerase IV in a warm-blooded animal such as a human being.

Thus according to a further aspect of the invention there is provided the use of a compound of formula (I) or (IA), or a pharmaceutically acceptable salt thereof, in the manufacture of a medicament for use in the treatment of a bacterial infection in a warm-blooded animal such as a human being.

Thus according to a further aspect of the invention there is provided the use of a compound of formula (I) or (IA), or a pharmaceutically acceptable salt thereof, in the manufacture of a medicament for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in a warm-blooded animal such as a human being.

According to a further aspect of the invention there is provided a compound of formula (I) or (IA), or a pharmaceutically acceptable salt thereof, for use in the production of an anti-bacterial effect in a warm-blooded animal such as a human being.

According to a further aspect of the invention there is provided a compound of formula (I) or (IA), or a pharmaceutically acceptable salt thereof, for use in inhibition of bacterial DNA gyrase and/or topoisomerase IV in a warm-blooded animal such as a human being.

Thus according to a further aspect of the invention there is provided a compound of formula (I) or (IA), or a pharmaceutically acceptable salt thereof, for use in the treatment of a bacterial infection in a warm-blooded animal such as a human being.

Thus according to a further aspect of the invention there is provided a compound of formula (I) or (IA), or a pharmaceutically acceptable salt thereof, for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in a warm-blooded animal such as a human being.

In order to use a compound of the formula (I) or (IA), or a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I) or (IA), or a pharmaceutically-acceptable salt thereof, and a pharmaceutically-acceptable diluent or carrier.

According to a further aspect of the invention there is provided a pharmaceutical composition which comprises a compound of formula (I) or (IA), as defined hereinbefore or a pharmaceutically acceptable salt thereof, in association with a pharmaceutically acceptable excipient or carrier for use in producing an anti-bacterial effect in an warm-blooded animal, such as a human being.

According to a further aspect of the invention there is provided a pharmaceutical composition which comprises a compound of formula (I) or (IA), as defined hereinbefore or a pharmaceutically acceptable salt thereof, in association with a pharmaceutically acceptable excipient or carrier for use in inhibition of bacterial DNA gyrase and/or topoisomerase IV in an warm-blooded animal, such as a human being.

According to a further aspect of the invention there is provided a pharmaceutical composition which comprises a compound of formula (I) or (IA), as defined hereinbefore or a pharmaceutically acceptable salt thereof, in association with a pharmaceutically acceptable excipient or carrier for use in the treatment of a bacterial infection in a warm-blooded animal, such as a human being.

According to a further aspect of the invention there is provided a pharmaceutical composition which comprises a compound of formula (I) or (IA), as defined hereinbefore or a pharmaceutically acceptable salt thereof, in association with a pharmaceutically acceptable excipient or carrier for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in an warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:

i) other antibacterial agents for example macrolides e.g. erythromycin, azithromycin or clarithromycin; quinolones e.g. ciprofloxacin or levofloxacin; B-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or

ii) anti-infective agents for example, an antifungal triazole e.g. or amphotericin; and/or

iii) biological protein therapeutics for example antibodies, cytokines, bactericidal/permeability-increasing protein (BPI) products; and/or

iv) efflux pump inhibitors.

Therefore, in a further aspect of the invention there is provided a compound of the formula (I) or (IA), or a pharmaceutically acceptable salt thereof, and a chemotherapeutic agent selected from:

i) one or more additional antibacterial agents; and/or

ii) one or more anti-infective agents; and/or

iii) biological protein therapeutics for example antibodies, cytokines, bactericidal/permeability-increasing protein (BPI) products; and/or

iv) one or more efflux pump inhibitors.

In another embodiment, the invention relates to a method of treating a bacterial infection in an animal, such as a human, comprising administering to the animal an effective amount of a compound of formula (I) or (IA), or a pharmaceutically acceptable salt thereof, and a chemotherapeutic agent selected from:

i) one or more additional antibacterial agents; and/or

ii) one or more anti-infective agents; and/or

iii) biological protein therapeutics for example antibodies, cytokines, bactericidal/permeability-increasing protein (BPI) products; and/or

iv) one or more efflux pump inhibitors.

As stated above the size of the dose required for the therapeutic or prophylactic treatment of a particular disease state will necessarily be varied depending on the host treated, the route of administration, the severity of the illness being treated, and whether or not an additional chemotherapeutic agent is administered in combination with a compound of the invention. Preferably a daily dose in the range of 1-50 mg/kg is employed. However the daily dose will necessarily be varied depending upon the host treated, the particular route of administration, the severity of the illness being treated, and whether or not an additional chemotherapeutic agent is administered in combination with a compound of the invention. Accordingly the optimum dosage may be determined by the practitioner who is treating any particular patient.

As noted above, one embodiment of the present invention is directed to treating or preventing diseases caused by bacterial infections, wherein the bacteria comprise a GyrB ATPase or topoisomerase IV ATPase enzyme. “Treating a subject with a disease caused by a bacterial infection” includes achieving, partially or substantially, one or more of the following: the reducing or amelioration of the progression, severity and/or duration of the infection, arresting the spread of an infection, ameliorating or improving a clinical symptom or indicator associated with a the infection (such as tissue or serum components), and preventing the reoccurrence of the infection.

As used herein, the terms “preventing a bacterial infection” refer to the reduction in the risk of acquiring the infection, or the reduction or inhibition of the recurrence of the infection. In a preferred embodiment, a compound of the invention is administered as a preventative measure to a patient, preferably a human, before a surgical procedure is preformed on the patient to prevent infection.

As used herein, the term “effective amount” refers to an amount of a compound of this invention for treating or preventing a bacterial infection is an amount which is sufficient to prevent the onset of an infection, reduce or ameliorate the severity, duration, or progression, of an infection, prevent the advancement of an infection, cause the regression of an infection, prevent the recurrence, development, onset or progression of a symptom associated with an infection, or enhance or improve the prophylactic or therapeutic effect(s) of another therapy.

In addition to its use in therapeutic medicine, compounds of formula (I) or (IA), and their pharmaceutically acceptable salts, are also useful as pharmacological tools in the development and standardization of in-vitro and in-vivo test systems for the evaluation of the effects of inhibitors of DNA gyrase and/or topoisomerase IV in laboratory animals such as cats, dogs, rabbits, monkeys, rats and mice, as part of the search for new therapeutic agents.

In the above other, pharmaceutical composition, process, method, use and medicament manufacture features, the alternative and particular embodiments of the compounds of the invention described herein also apply.

### Example

The invention is now illustrated but not limited by the following Example in which unless otherwise stated:—

(i) evaporations were carried out by rotary evaporation in-vacuo and work-up procedures were carried out after removal of residual solids by filtration;
(ii) operations were generally carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable; the structure of the end-products of the invention were generally confirmed by NMR and mass spectral techniques; proton magnetic resonance spectra is quoted and was generally determined in DMSO-d₆ unless otherwise stated using a Bruker DRX-300 spectrometer operating at a field strength of 300 MHz. Chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad; fast-atom bombardment (FAB) mass spectral data were generally obtained using a Platform spectrometer (supplied by Micromass) run in electrospray and, where appropriate, either positive ion data or negative ion data were collected or using Agilent 1100series LC/MSD equipped with Sedex 75ELSD, run in atmospheric pressure chemical ionization mode and, where appropriate, either positive ion data or negative ion data were collected; mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported;
(vi) each intermediate was purified to the standard required for the subsequent stage and was characterized in sufficient detail to confirm that the assigned structure was correct; purity was assessed by high pressure liquid chromatography, thin layer chromatography, or NMR and identity was determined by infra-red spectroscopy (IR), mass spectroscopy or NMR spectroscopy as appropriate;
(vii) the following abbreviations may be used:

ACN is acetonitrile;

CDCl₃ is deuterated chloroform;

DBU is 1,8-diazabicyclo[5.4.0]undec-7-ene;

DCM is dichloromethane;

DIEA is diisopropyl ethylamine;

DMF is N,N-dimethylformamide;

DMSO is dimethylsulfoxide;

EDC is 1-ethyl-3-(3-dimethyllaminopropyl)carbodiimide;

EtOAc is ethyl acetate;

EtOH is ethanol;

HATU is N-[(dimethylamino)-1H,2,3-triazolo[4,5-b-]pyridin-1-ylmethylene]-N-methylmethanaminium hexafluorophosphate N-oxide;

HOBT is 1-hydroxybenzotriazole;

MeOH is methanol;

MS is mass spectroscopy;

RT or rt is room temperature;

SM is starting material;

TBAF is tetra-n-butylammonium fluoride;

TFA is trifluoroacetic acid;

TFAA is trifluoroacetic anhydride;

THF is tetrahydrofuran; and

(viii) temperatures are quoted as ° C.

### Examples 1-2

The compounds in the following table below were synthesized by the general procedure described below.

(S)-3-(di-tert-butoxyphosphoryloxy)propyl 6-(6-(3-ethylureido)-4-(4-(trifluoromethyl)thiazol-2-yl)pyridin-3-yl)-1-(1-hydroxy-3,3-dimethylbutan-2-yl)-4-oxo-1,4-dihydroquinoline-3-carboxylate (Intermediate 1) or (S)-2-(di-tert-butoxyphosphoryloxy)ethyl 6-(6-(3-ethylureido)-4-(4-(trifluoromethyl)thiazol-2-yl)pyridin-3-yl)-1-(1-hydroxy-3,3-dimethylbutan-2-yl)-4-oxo-1,4-dihydroquinoline-3-carboxylate (Intermediate 2) (0.88 mmol) was dissolved in 7 mL of dioxane. Anisole (0.2 mL, 1.83 mmol) was added. A 4 M solution of HCl in dioxane (2.0 mL, 8.00 mmol) was added dropwise via syringe. An additional 3 mL of dioxane was added. The reaction mixture was stirred at RT for 11 h. The volume of the reaction mixture was reduced by two-thirds. Dioxane (10 mL) was added and the volume of the reaction mixture was again reduced by two-thirds. Diethyl ether (18 mL) was added and the resultant solid was collected. The solid was dissolved in a minimal amount of MeOH, then several volumes of water were added to give a white suspension. The suspension was concentrated in vacuo and then dried by lyophilization.

| Ex | Compound | Data | SM |
|---|---|---|---|
| 1 | (S)-3-(phosphonooxy)propyl 6-(6- (3-ethylureido)-4-(4- (trifluoromethyl)thiazol-2- yl)pyridin-3-yl)-1-(1-hydroxy-3,3- dimethylbutan-2-yl)-4-oxo-1,4- dihydroquinoline-3-carboxylate 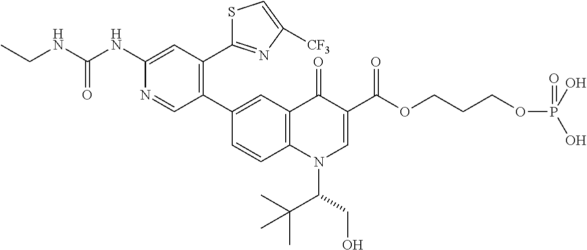 | LC/MS (ES⁺)[(M + H)⁺]: 742 for C₃₁H₃₅F₃N₅O₉PS ¹H NMR (DMSO-d₆): δ9.50 (s, 1H); 8.67 (s, 1H); 8.52 (s, 1H); 8.34 (s, 1H); 8.21 (m, 3H); 7.62 (m, 2H); 4.96 (m, 1H); 4.26 (m, 2H); 4.01 (m, 4H); 3.21 (m, 2H); 1.98 (m, 2H); 1.11 (t, 3H); 0.97 (s, 9H). | Intermediate 1 |
| 2 | (S)-2-(phosphonooxy)ethyl 6-(6- (3-ethylureido)-4-(4- (trifluoromethyl)thiazol-2- yl)pyridin-3-yl)-1-(1-hydroxy-3,3- dimethylbutan-2-yl)-4-oxo-1,4- dihydroquinoline-3-carboxylate 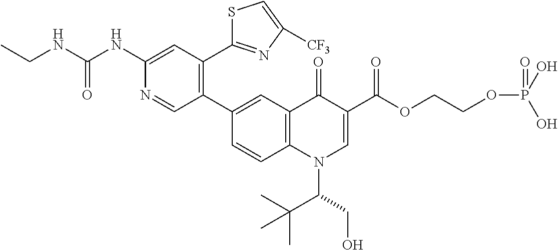 | LC/MS (ES⁺)[(M + H)⁺]: 728 for C₃₀H₃₃F₃N₅O₉PS ¹H NMR (DMSO-d₆): δ9.49 (s, 1H); 8.72 (s, 1H); 8.52 (m, 1H); 8.35 (s, 1H); 8.23 (m, 3H); 7.64 (m, 2H); 4.98 (m, 1H); 4.37 (m, 2H); 4.08 (m, 4H); 3.21 (m, 2H); 1.11 (t, 3H); 0.97 (s, 9H). | Intermediate 2 |

### Example 3

### (S)-1-(3,3-dimethyl-1-(phosphonooxy)butan-2-yl)-6-(6-(3-ethylureido)-4-(4-(trifluoromethyl)thiazol-2-yl)pyridin-3-yl)-4-oxo-1,4-dihydroquinoline-3-carboxylic acid

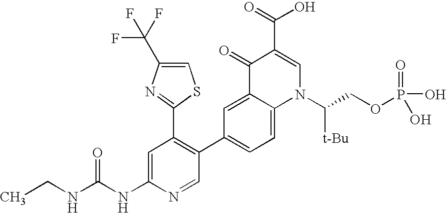

To a solution of 1-((2S)-1-(tert-butoxy(hydroxy)phosphoryloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylic acid (Intermediate 17, 528.4 mg, 0.71 mmol, 1 equiv.) in dichloromethane (6 mL) was added 4 M hydrogen chloride in 1,4-dioxane (1.25 mL, 5.0 mmol, 7 equiv.). The reaction mixture was stirred at room temperature for 1 h. The precipitate was collected by filtration and washed with diethyl ether then dried. The precipitate was purified by reverse phase HPLC(C18, 0-95% acetonitrile/water, gradient) to provide (S)-1-(3,3-dimethyl-1-(phosphonooxy)butan-2-yl)-6-(6-(3-ethylureido)-4-(4-(trifluoromethyl)thiazol-2-yl)pyridin-3-yl)-4-oxo-1,4-dihydroquinoline-3-carboxylic acid as a light yellow solid (333.7 mg, 69%).

Calcd for C₂₈H₂₉F₃N₅O₈PS[M+H]⁺: 683.93.

¹H NMR (d₆-DMSO) δ 9.49 (s, 1H), 8.82 (s, 1H), 8.5 (s, 1H), 8.48 (d, 1H), 8.37 (s, 1H), 8.3 (d, 1H), 8.2 (s, 1H), 7.81 (dd, 1H0, 7.63 (t, 1H), 7.15 (s, 2H), 5.41 (t, 1H), 7.39 (t, 2H), 3.25-3.17 (m, 2H), 1.11 (t, 3H), 0.97 (s, 9H).

### Examples 4-7

The following Examples were prepared according to the procedure described for Example 3 from the indicated starting material.

| Ex | Compound | Data | SM |
|---|---|---|---|
| 4 | (S)-6-(6-(3-ethylureido)-4-(4- (trifluoromethyl)thiazol-2-yl)pyridin-3- yl)-1-(3-methyl-1-(phosphonooxy)butan- 2-yl)-4-oxo-1,4-dihydroquinoline-3- carboxylic acid 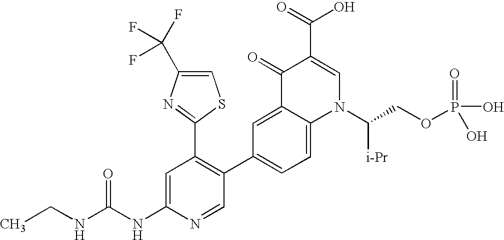 | Calcd for C₂₇H₂₇F₃N₅O₈PS [M + H]⁺: 669.92 ¹H NMR (d₆-DMSO) δ 9.49 (s, 1H), 8.93 (s, 1H), 8.48 (s, 1H), 8.38 (d, 1H), 8.37 (s, 1H), 8.31 (d, 1H), 8.22 (s, 1H), 7.84 (dd, 1H), 7.65 (t, 1H), 7.12 (s, 2H), 5.12-5.08 (m, 1H), 4.3-2.23 (m, 1H), 4.16-4.09 (m, 1H), 3.26-3.17 (m, 2H), 2.45-2.28 (m, 1H), 1.14 (d, 3H), 1.11 (t, 3H), 0.73 (d, 3H) | Intermediate 18 |
| 5 | (S)-6-(6-(3-ethylureido)-4-(4- (trifluoromethyl)thiazol-2-yl)pyridin-3- yl)-4-oxo-1-(1-(phosphonooxy)butan-2- yl)-1,4-dihydroquinoline-3-carboxylic acid 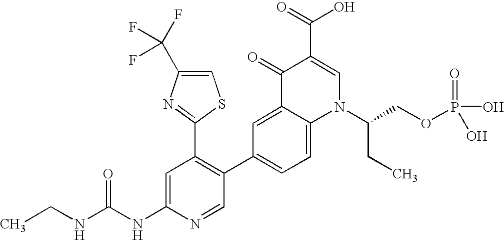 | Calcd for C₂₆H₂₅F₃N₅O₈PS [M + H]⁺: 655.90 ¹H NMR (d₆-DMSO) δ 9.49 (s, 1H), 8.88 (s, 1H), 8.47 (s, 1H), 8.36 (s, 1H), 8.31 (d, 1H), 8.23 (s, 1H), 7.87 (dd, 1H), 7.66 (t, 1H), 7.14 (s, 2H), 5.35-5.29 (m, 1H), 4.2- 4.14 (m, 1H), 4.11-4.04 (m, 1H), 3.26-3.16 (m, 2H), 2.13- 1.93 (m, 2H), 1.11 (t, 3H), 0.86 (t, 3H) | Intermediate 19 |
| 6 | (R)-6-(6-(3-ethylureido)-4-(4- (trifluoromethyl)thiazol-2-yl)pyridin-3- yl)-4-oxo-1-(1-(phosphonooxy)butan-2- yl)-1,4-dihydroquinoline-3-carboxylic acid 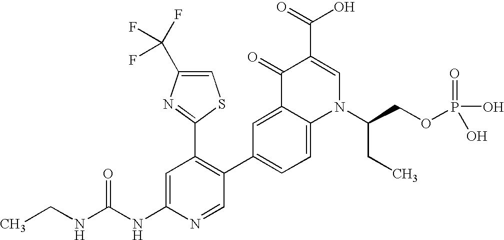 | Calcd for C₂₆H₂₅F₃N₅O₈PS [M + H]⁺: 655.97 ¹H NMR (d₆-DMSO) δ 9.51 (s, 1H), 8.88 (s, 1H), 8.47 (s, 1H), 8.38 (d, 1H), 8.34 (s, 1H), 8.3 (d, 1H), 8.23 (s, 1H), 7.86 (dd, 1H), 7.68 (t, 1H), 7.17 (s, 2H), 5.35-5.26 (m, 1H), 4.19-4.0 (m, 2H), 3.25- 3.19 (m, 2H), 2.12-1.89 (m, 2H), 1.11 (t, 3H), 0.86 (t, 3H) | Intermediate 20 |
| 7 | (5)-6-(6-(3-ethylureido)-4-(4- (trifluoromethyl)thiazol-2-yl)pyridin-3- yl)-1-(4-methyl-1-(phosphonooxy)pentan- 2-yl)-4-oxo-1,4-dihydroquinoline-3- carboxylic acid 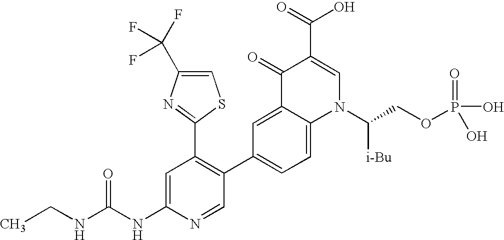 | Calcd for C₂₈H₂₉F₃N₅O₈PS [M + H]⁺: 684.01 ¹H NMR (d₆-DMSO) δ 9.57 (s, 1H), 8.87 (s, 1H), 8.5 (s, 1H), 8.45 (d, 1H), 8.32 (s, 1H), 8.29 (d, 1H), 8.24 (s, 1H), 7.84 (dd, 1H), 7.71 (t, 1H), 5.45-5.4 (m, 1H), 4.05- 4.1 (m, 2H), 3.24-3.18 (m, 2H), 1.99-1.83 (m, 2H), 1.47- 1.4 (m, 1H), 1.1 (t, 3H), 0.9 (d, 3H), 0.83 (d, 3H) | Intermediate 21 |

### Example 8

### 2-(1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxamido)ethyl dihydrogen phosphate hydrochloride

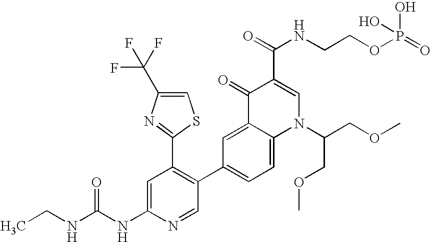

To a solution of 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylic acid (Intermediate 43, 150 mg, 0.25 mmol) in dimethyl formamide (1 mL) was added HATU (124.5 mg, 0.33 mmol) and the dark brown solution stirred at room temperature for 5 min. To this was added a solution of 2-aminoethyl di-tert-butyl phosphate (Intermediate 37, 164.5 mg, 0.65 mmol) in dichloromethane (2 mL) followed by diisopropyl ethyl amine (130.7 μL, 0.75 mmol). Water (5 mL) was added and the mixture was extracted with dichloromethane (4×, 5 mL). The organic layers were combined and washed with saturated sodium bicarbonate, then dried over sodium sulfate and the solvent was evaporated. The residue was purified by Analogix (2×, methanol: dichloromethane) to give 160 mg of clean material plus some impure fractions.

The clean product (˜90 mg, 11 mmol) was deprotected by dissolving it in dichloromethane (2 mL) then adding 4M HCl in dioxane (2 mL). The mixture was stirred for ˜30 min, then the volatiles were removed in vacuo and the residue was dissolved in water and lyophilized to a light yellow solid.

MS (ESP): 729 (MH⁺) for C₂₉H₃₃ClF₃N₆O₉PS ¹H NMR (300 MHz, CD₃OD): δ 1.22 (t, 3H), 3.37 (m, 2H), 3.71 (s, 6H), 3.73 (t, 2H), 3.88 (m, 2H), 3.97 (m, 2H), 4.15 (m, 2H), 5.38 (m, 1H), 7.71 (d, 1H), 7.89 (s, 1H), 8.03 (d, 1H), 8.15 (s, 1H), 8.35 (s, 1H), 8.42 (s, 1H), 9.08 (s, 1H).

### Examples 9-11

The following Examples were prepared according to the procedure described for Example 8 from the starting materials indicated.

| Ex | Compound | Data | SM |
|---|---|---|---|
| 9 | 2-(1-cyclopropyl-6-(6-(3-ethylureido)-4- (4-(trifluoromethyl)thiazol-2-yl)pyridin-3- yl)-4-oxo-1,4-dihydroquinoline-3- carboxamido)ethyl dihydrogen phosphate hydrochloride 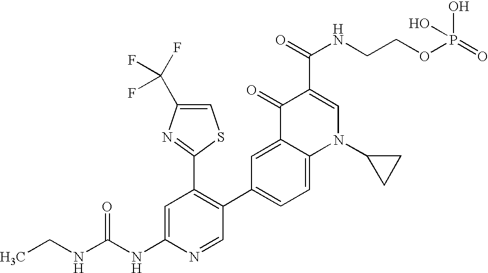 | MS (ESP): 667 (MH⁺) (for C₂₇H₂₇ClF₃N₆O₇PS ¹H NMR (300 MHz, CD₃OD): δ 1.22 (m, 4H), 1.30 (m, 1H), 1.38 (m, 2H), 3.34 (q, 2H), 3.71 (m, 3H), 4.12 (m, 2H), 7.73 (d, 1H), 7.89 (s, 1H), 8.15 (s, 1H), 8.25 (d, 1H), 8.33 (s, 1H), 8.35 (s, 1H), 8.91 (s, 1H). | Intermediate 44 and Intermediate 37 |
| 10 | 3-(1-(1,3-dimethoxypropan-2-y1)-6-(6-(3- ethylureido)-4-(4-(trifluoromethyl)thiazol- 2-yl)pyridin-3-yl)-4-oxo-1,4- dihydroquinoline-3-carboxamido)propyl dihydrogen phosphate hydrochloride 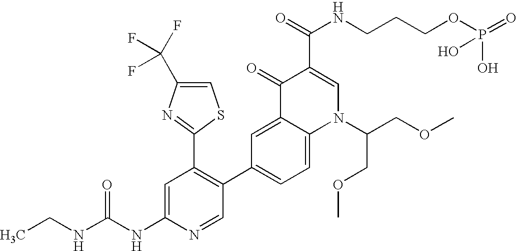 | MS (ESP): 743 (MH⁺) (for C₃₀H₃₅ClF₃N₆O₉P₅ ¹H NMR (300 MHz, CD₃OD): δ 1.22 (t, 3H), 2.02 (m, 2H), 3.34 (s, 6H), 3.37 (m 2H) 3.58 (t, 2H) 3.88 (m, 2H), 3.97 (m, 2H), 4.10 (q, 2H), 7.71 (d, 1H), 7.94 (s, 1H), 8.09 (d, 1H), 8.19 (s 1H), 8.34 (s, 1H), 8.45 (s, 1H), 9.09 (s, 1H). | Intermediate 43 and Intermediate 38 |
| 11 | 3-(1-cyclopropyl-6-(6-(3-ethylureido)-4- (4-(trifluoromethyl)thiazol-2-yl)pyridin-3- yl)-4-oxo-1,4-dihydroquinoline-3- carboxamido)propyl dihydrogen phosphate hydrochloride 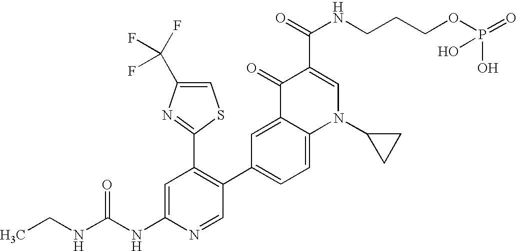 | MS (ESP): 6801 (MH⁺) for C₂₈H₂₉ClF₃N₆O₇P₅ ¹H NMR (300 MHz, CD₃OD): δ 1.23 (m, 5H), 1.40 (m, 2H), 2.00 (m, 2H), 3.35 (m, 2H), 3.56 (t, 2H), 3.79 (m, 1H), 4.09 (q, 2H), 7.84 (d, 1H), 8.11 (s, 1H), 8.31 (s, 1H), 8.35 (s, 1H), 8.38 (d, 1H), 8.49 (s, 1H), 8.93 (s, 1H). | Intermediate 44 and Intermediate 38 |

### Example 12

### (S)-6-(4-(4-cyclopropylthiazol-2-yl)-6-(3-ethylureido)pyridin-3-yl)-1-(4-methyl-1-(phosphonooxy)pentan-2-yl)-4-oxo-1,4-dihydroquinoline-3-carboxylic acid

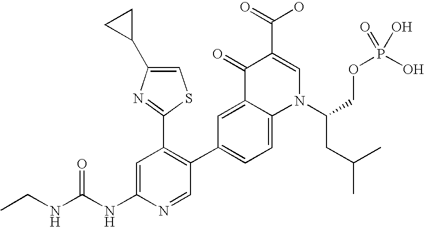

In a 25 mL round-bottom flask, (S)-6-(4-(4-cyclopropylthiazol-2-yl)-6-(3-ethylureido)pyridin-3-yl)-1-(1-(di-tert-butoxyphosphoryloxy)-4-methylpentan-2-yl)-4-oxo-1,4-dihydroquinoline-3-carboxylic acid (Intermediate 48, 0.102 g, 0.13 mmol) was dissolved in 1,4-dioxane (1.5 mL). 4M HCl in dioxane (0.032 mL, 0.93 mmol) was added, and the reaction was stirred at room temperature overnight. The reaction mixture was concentrated under reduced pressure. The resulting solids were triturated in ether. The solid was collected by filtration and washed with ether. Isolation gave 75 mg of the title compound as an off-white solid.

LC/MS (ES⁺)[(M+H)⁺]: 656 for C₃₀H₃₄N₅O₈PS.

¹H NMR (300 MHz, d₆-DMSO): 0.4 (m, 2H), 0.71 (m, 2H), 0.89 (d, 3H), 0.91 (d, 3H), 1.11 (t, 3H), 1.47 (m, 1H), 1.93 (m, 2H), 2.03 (m, 1H), 3.22 (m, 2H), 4.18 (m, 1H), 4.28 (m, 1H), 5.44 (m, 1H), 7.34 (s, 1H), 7.79 (m 1H), 7.82 (m, 1H), 8.08 (s, 1H), 8.26 (m, 1H), 8.29 (s, 1H), 8.32 (m, 1H), 8.89 (s, 1H), 9.43 (s, 1H).

### Example 13

### 3-{[(7-{6[(ethylcarbamoyl)amino]-4-[4-(trifluoromethyl)-1,3-thiazol-2-yl]pyridin-3-yl}-1-[(5-methyl-1,2,4-oxadiazol-3-yl)methyl]-4-oxo-1,4-dihydroquinolin-3-yl)carbonyl]amino}propyl dihydrogen phosphate

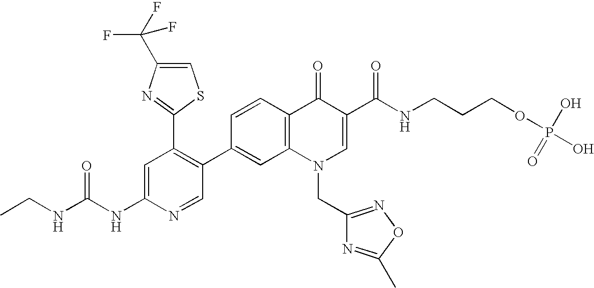

To a stirred solution of 7-{6-[(ethylcarbamoyl)amino]-4-[4-(trifluoromethyl)-1,3-thiazol-2-yl]pyridin-3-yl}-1-[(5-methyl-1,2,4-oxadiazol-3-yl)methyl]-4-oxo-1,4-dihydroquinoline-3-carboxylic acid (Intermediate 53, 0.1 g, 0.166 mmol) in dimethylformamide (4 mL) was added triethylamine (0.07 mL, 0.499 mM) and 2-(1H-7-azabenzotriazol-1-yl)-1,1,3,3-tetramethyl uronium hexafluorophosphate methanaminium (HATU) (0.095 g, 0.249 mM), and the mixture was stirred at room temperature for 10 min. 3-Aminopropyl dihydrogen phosphate (Intermediate 50, 0.038 g, 0.249 mmol) was added to the mixture, and it was stirred at room temperature for 2 h. After the completion of the reaction, the reaction mixture was concentrated to dryness. To the residue, water (10 mL) was added and the mixture was stirred for 10 min. The solid which precipitated out was filtered and dried then taken up in methanol (5 mL) and acetonitrile (2 mL) and stirred for 10 min. The solid was collected by filtration and dried to obtain 0.035 g (28.6%) of 3-{[(7-{6-[(ethylcarbamoyl)amino]-4-[4-(trifluoromethyl)-1,3-thiazol-2-yl]pyridin-3-yl}-1-[(5-methyl-1,2,4-oxadiazol-3-yl)methyl]-4-oxo-1,4-dihydroquinolin-3-yl)carbonyl]amino}propyl dihydrogen phosphate as an off-white solid.

¹H NMR (400 MHz, DMSO-d₆): δ 1.10 (t, 3H), 1.82 (m, 2H), 3.19-3.22 (q, 2H), 3.32-3.42 (m, 2H), 3.85 (m, 2H), 5.93 (s, 2H), 7.44-7.46 (m, 2H), 7.57 (m, 1H), 7.82 (s, 1H), 8.24-8.28 (m, 2H), 8.36-8.41 (m, 2H), 9.07 (s, 1H).

LC-MS: m/z 737.4 (M+H).

### Example 14

### 3-{[(1-ethyl-7-{6-[(ethylcarbamoyl)amino]-4-[4-(trifluoromethyl)-1,3-thiazol-2-yl]pyridin-3-yl}-4-oxo-1,4-dihydroquinolin-3-yl)carbonyl]amino}propyl dihydrogen phosphate

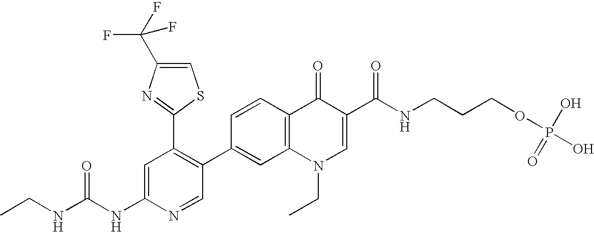

To a stirred solution of 1-ethyl-7-{6-[(ethylcarbamoyl)amino]-4-[4-(trifluoromethyl)-1,3-thiazol-2-yl]pyridin-3-yl}-4-oxo-1,4-dihydroquinoline-3-carboxylic acid (Intermediate 58, 300 mg, 0.56 mmol) in dimethylformamide (20 mL) at room temperature was added triethylamine (0.24 mL, 1.69 mmol) and HATU (436 mg, 1.12 mmol) and the mixture was stirred for 1 h, then 3-aminopropyl dihydrogen phosphate (Intermediate 50, 175 mg, 1.12 mmol) was added. After completion of the reaction, the reaction mixture was concentrated to dryness under reduced pressure, then water (30 mL) was added to the residue and the mixture was stirred for 20 min. The mixture was filtered and the solid which was collected was stirred in methanol (20 mL), filtered and washed with diethyl ether (20 mL) to afford 320 mg, (84.8%) of the crude product. The crude product was purified by preparative HPLC(C18, 0-95% acetonitrile/water/ammonium acetate, gradient) to afford 85 mg (22.5%) of 3-{[(1-ethyl-7-{6-[(ethylcarbamoyl)amino]-4-[4-(trifluoromethyl)-1,3-thiazol-2-yl]pyridin-3-yl}-4-oxo-1,4-dihydroquinolin-3-yl)carbonyl]amino}propyl dihydrogen phosphate.

¹H NMR (400 MHz, DMSO-d₆): δ 1.09 (t, 3H), 1.20 (t, 3H), 1.79 (s, 2H), 3.80 (s, 3H), 4.46 (d, 2H), 7.42 (d, 2H), 7.61 (s, 1H), 7.88 (s, 1H), 8.21 (s, 1H), 8.38 (m, 4H), 8.88 (s, 1H), 9.47 (s, 1H), 9.93 (s, 1H).

LC-MS: m/z 669.4 (M+H).

### Examples 15-17

The following Examples were prepared according to the procedure described for Example 14 from the starting material indicated in the table.

| Ex | Compound | Data | SM |
|---|---|---|---|
| 15 | 2-(1-cyclopropyl-6-(6-(3-ethylureido)-4- (4-(trifluoromethyl)thiazol-2-yl)pyridin- 3-yl)-4-oxo-1,4-dihydroquinoline-3- carboxamido)ethyl dihydrogen phosphate hydrochloride 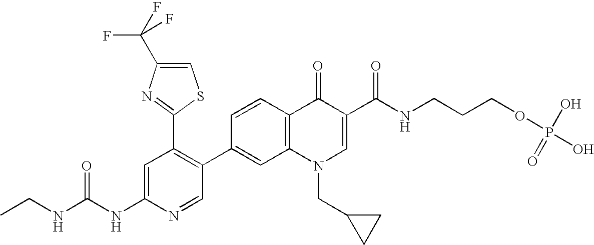 | ¹H NMR (400 MHz, DMSO-d₆): δ 0.37 (dd, 4H), 1.01 (s, 1H), 1.10 (t, 3H), 1.80 (s, 2H), 3.83 (s, 3H), 4.30 (s, 2H), 7.45 (m, 1H), 7.61 (s, 1H), 7.96 (s, 1H), 8.20 (s, 1H), 8.39 (m, 4H), 8.89 (s, 1H), 9.46 (s, 1H), 9.94 (s, 1H). LC-MS: m/z 695.4 (M + H) | Intermediate 61 and Intermediate 50 |
| 16 | 3-({[7-{ 6-[(ethylcarbamoyl)amino]-4-[4- (trifluoromethyl)-1,3-thiazol-2-yl]pyridin- 3-yl}-1-(3-methylbutyl)-4-oxo-1,4- dihydroquinolin-3- yl]carbonyl}amino)propyl dihydrogen phosphate 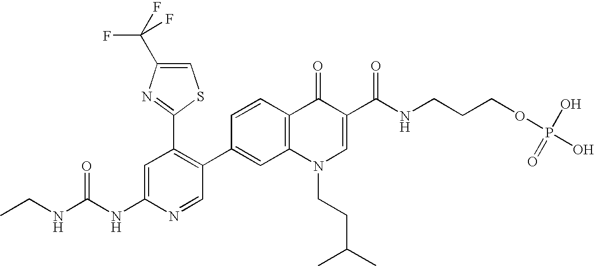 | ¹H NMR (400 MHz, DMSO-d₆) δ 0.80 (d, 6H), 1.10 (m, 6H), 1.39 (s, 4H), 1.79 (s, 3H), 2.97 (s, 2H), 3.17 (d, 4H), 3.82 (s, 3H), 4.39 (s, 2H), 7.48 (m, 3H), 7.76 (s, 1H), 8.23 (s, 1H), 8.36 (m, 3H), 8.85 (s, 1H), 9.46 (s, 1H), 9.92 (s, 1H). LC-MS: m/z 711.4 (M + H) | Intermediate 50 and Intermediate 64 |
| 17 | 3-{[(7-{6-[(ethylcarbamoyl)amino]-4-[4- (trifluoromethyl)-1,3-thiazol-2- yl]pyridin-3-yl}-4-oxo-1-propyl-1,4- dihydroquinolin-3- yl)carbonyl]amino}propyl dihydrogen phosphate 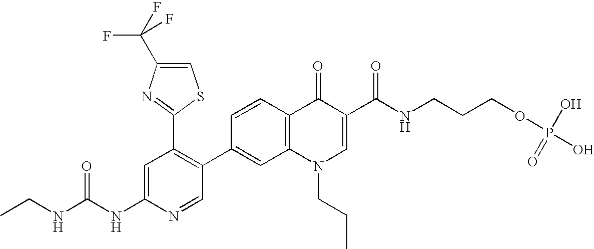 | ¹H NMR (400 MHz, DMSO-d₆): δ 0.77 (s, 2H), 1.11 (d, 3H), 1.53 (s, 2H), 1.81 (s, 2H), 3.02 (s, 1H), 3.41 (m, 4H), 3.83 (s, 4H), 4.36 (s, 2H), 7.47 (d, 2H), 7.83 (s, 1H), 8.20 (s, 1H), 8.38 (m, 3H), 8.85 (s, 1H), 9.47 (s, 1H), 9.94 (s, 1H). LC-MS: m/z 683.4 (M + H). | Intermediate 50 and Intermediate 67 |

### Example 18

### 2-(phosphonooxy)ethyl 1-cyclopropyl-6-(6-(3-ethylureido)-4-(4-(trifluoromethyl)thiazol-2-yl)pyridin-3-yl)-4-oxo-1,4-dihydroquinoline-3-carboxylate hydrochloride

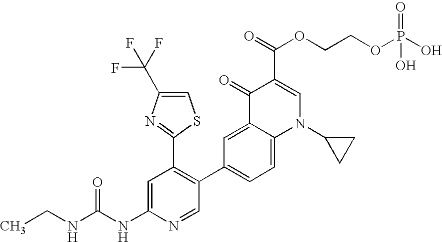

To a suspension of 1-cyclopropyl-6-(6-(3-ethylureido)-4-(4-(trifluoromethyl)thiazol-2-yl)pyridin-3-yl)-4-oxo-1,4-dihydroquinoline-3-carboxylic acid (Intermediate 44, 150 mg, 0.25 mmol) in dimethyl formamide (1 mL) was added HATU (124.3 mg, 0.32 mmol) and mixture became clear solution. After ˜5 min di-tert-butyl 2-hydroxyethyl phosphate (Intermediate 70, 126.8 mg, 0.50 mmol) was added followed by 4-(N,N-dimethylamino)-pyridine (24.4 mg, 0.05 mmol) and diisopropyl ethylamine (130.5 μL, 0.75 mmol). After the reaction was complete, it was filtered (PTFE 4 um filter) and the filtrate was concentrated to a residue. The residue was re-dissolved in dichloromethane (0.5 mL) and treated with excess 4M HCl in dioxane to remove the tert-butyl protecting groups. The reaction was complete in ˜30 min. The volatiles were removed under vacuum and the residue was dissolved in acetonitrile/water and lyophilized to give the product.

MS (ESP): 668 (M+H⁺) for C₂₇H₂₆ClF₃N₅O₈PS.

¹H NMR (300 MHz, DMSO-d₆): δ 1.13 (m, 5H), 1.26 (m, 2H), 3.21 (q, 2H), 3.72 (m, 1H), 4.14 (m, 2H), 4.35 (m, 2H), 7.65 (brs, 1H), 7.79 (d, 1H), 8.14 (s, 1H), 8.18 (d, 1H), 8.24 (s, 1H), 8.32 (s, 1H), 8.49 (s, 1H), 8.65 (s, 1H), 8.94 (s, 1H).

### Example 19

### 2-(phosphonooxy)ethyl 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylate

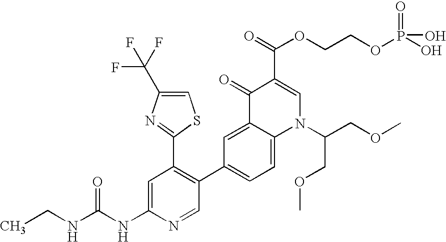

To a solution of 2-(di-tert-butoxyphosphoryloxy)ethyl 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylate (Intermediate 71, 1.2 g, 1.4 mmol) in dichloromethane (30 mL) was added 4M hydrochloric acid in dioxane (2 mL) and the mixture stirred at room temperature for 30 min. The solvent was removed and the product was dissolved in dichloromethane and then concentrated under vacuum twice to remove excess HCl. The residue was then partitioned between dichloromethane and water and the product formed a solid at the interface. The mixture was filtered, and the pale yellow solids dried under vacuum to give 840 mg product.

MS (ESP): 730.2M+H⁺) for C₂₉H₃₁F₃N₅O₁₀PS

¹H NMR (300 MHz, CD₃OD): δ 1.23 (t, 3H), 3.37 (s, 6H), 3.90 (m, 2H), 3.97 (m, 2H), 4.32 (m, 2H), 4.50 (br, 2H), 5.33 (m, 1H), 7.69 (d, 1H), 7.90 (s, 1H), 8.02 (d, 1H), 8.17 (s, 1H), 8.34 (brs, 1H), 8.43 (s, 1H), 9.06 (s, 1H).

### Example 20

### 1-(2-(dimethylamino)ethyl)-6-(6-(3-ethylureido)-4-(4-(trifluoromethyl)thiazol-2-yl)pyridin-3-yl)-7-fluoro-4-oxo-1,4-dihydroquinoline-3-carboxylic acid

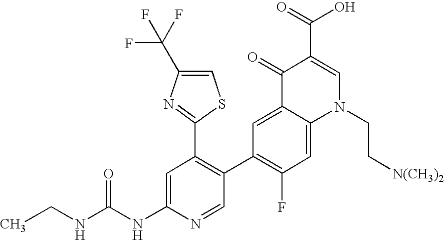

To a solution of palladium (II) acetate (22.95 mg, 0.10 mmol, 0.1 equiv.) and 1,1′-bis(di-t-butylphosphino)ferrocene (48.5 mg, 0.10 mmol, 0.1 equiv.) in acetonitrile (3 mL) was added ethyl 2-(2,4-difluoro-5-iodobenzoyl)-3-(dimethylamino)acrylate (Intermediate 82, 400 mg, 1.02 mmol, 1 equiv.), followed by 6-(3-ethylureido)-4-(4-(trifluoromethyl)thiazole-2-yl)pyridine-3-ylboronic acid (WO2009106885, 372 mg, 1.03 mmol, 1.01 equiv.) and a solution of potassium carbonate (212 mg, 1.53 mmol, 1.5 equiv.) in water (1 mL). The reaction mixture was stirred at 60° C. for 9 h. The reaction mixture was concentrated under reduced pressure and re-suspended in 1,4-dioxane (3 mL). 2 M lithium hydroxide (1.02 mL) was added, and the reaction mixture was stirred at 100° C. for 4 h. The reaction mixture was cooled to room temperature and diluted with water. 1 N HCl was added until pH 3-4 was reached. The precipitate was washed with water and hexanes and dried. The compound was purified (silica gel chromatography) and concentrated to provide 1-(2-(dimethylamino)ethyl)-6-(6-(3-ethylureido)-4-(4-(trifluoromethyl)thiazol-2-yl)pyridin-3-yl)-7-fluoro-4-oxo-1,4-dihydroquinoline-3-carboxylic acid (90.7 mg, 15%).

Calcd for C₂₆H₂₄F₄N₆O₄S [M+H]⁺: 593.16.

H¹ NMR (d₆-DMSO) δ 14.92 (s, 1H), 9.53 (s, 1H), 9.00 (s, 1H), 8.58 (s, 1H), 8.45 (d, 1H), 8.39 (s, 1H), 8.27 (s, 1H), 8.05 (d, 1H), 7.54 (t, 1H), 4.73-4.69 (m, 2H), 3.26-3.17 (m, 2H), 2.49 (s, 6H), 2.40-2.27 (m, 2H), 1.11 (t, 3H).

### Examples 21-27

The following Examples were prepared according to the procedure described for Example 20 from the starting material indicated in the table.

| Ex | Compound | Data | SM |
|---|---|---|---|
| 21 | 6-(6-(3-ethylureido)-4-(4- (trifluoromethyl)thiazol-2-yl)pyridin-3- yl)-7-fluoro-1-((S)-1-hydroxy-3- methylbutan-2-yl)-4-oxo-1,4- dihydroquinoline-3-carboxylic acid 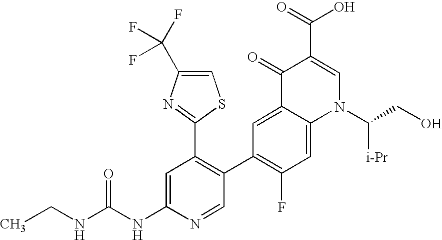 | Calcd for C₂₈H₂₇F₄N₅O₅S. H¹NMR (d₆-DMSO) δ 14.98 (s, 1H), 9.54 (s, 1H), 8.89 (s, 1H), 8.59 (s, 1H), 8.47 (d, 1H), 8.41 (s, 1H), 8.26 (s, 1H), 8.25 (s, 1H), 7.54 (t, 1H), 5.25-5.22 (m, 2H), 5.20- 5.09 (m, 2H), 3.83-3.67 (m, 2H), 3.26-3.17 (m, 2H), 2.02- 1.76 (m, 2H), 1.47-1.42 (m, 1H), 1.11 (t, 3H), 0.92 (d, 3H), 0.87 (d, 3H) | Intermediate 75 and 6-(3- ethylureido)4-(4- (trifluoromethyl) thiazole 2- yl)pyridine- 3-ylboronic acid (WO2009106885) |
| 22 | 1-ethyl-6-(6-(3-ethylureido)-4-(4- (trifluoromethyl)thiazol-2-yl)pyridin-3- yl)-7-(4-methylpiperazin-1-yl)-4-oxo-1,4- dihydroquinoline-3-carboxylic acid 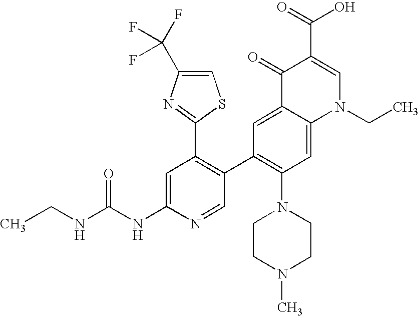 | Calcd for C₂₉H₃₀F₃N₇O₄S [M + H]⁺: 630.21. NMR (d₆-DMSO) δ 15.25 (s, 1H), 9.54 (s, 1H), 9.03 (s, 1H), 9.43 (s, 1H), 8.41 (s, 1H), 8.26 (s, 1H), 8.24 (s, 1H), 7.71 (t, 1H), 7.23 (s, 1H), 4.66-4.63 (m, 2H), 3.26- 3.17 (m, 2H), 2.90-2.85 (m, 2H), 2.84-2.5 (m, 4H), 2.45- 2.32 (m, 2H), 1.43 (t, 3H), 1.11 (t, 3H). | Intermediate 72 and 6-(3- ethylureido)- 4-(4- (trifluoromethyl) thiazole- 2- yl)pyridine- 3-ylboronic acid (WO2009106885) |
| 23 | 7-(2-(dimethylamino)ethoxy)-1-(((S)-1- ethylpyrrolidin-2-yl)methyl)-6-(6-(3- ethylureido)-4-(4-phenylthiazol-2- yl)pyridin-3-yl)-4-oxo-1,4- dihydroquinoline-3-carboxylic acid 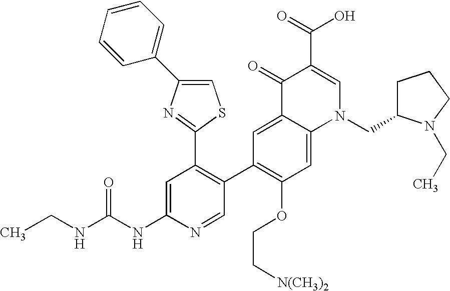 | Calcd for C₃₈H₄₃N₇O₅S [M + H]⁺: 710.35. H¹NMR (d₆-DMSO) δ 15.39 (s, 1H), 9.39 (s, 1H), 8.92 (s, 1H), 8.28 (s, 2H), 8.23 (s, 1H), 8.13 (s, 1H), 8.14 (s, 1H), 7.75-7.70 (m, 2H), 7.39- 7.33 (m, 5H), 4.65-4.61 (m, 1H), 4.41-4.38 (m, 1H), 4.08- 4.00 (m, 2H), 3.26-3.17 (m, 2H), 3.07-3.05 (m, 1H), 3.02- 3.00 (m, 1H), 2.22-2.09 (m, 4H), 1.93 (s, 6H), 1.71-1.68 (m, 2H), 1.55-1.50 (m, 2H), 1.24-1.22 (m, 2H), 1.12 (t, 3H), 0.76 (t, 3H) | Intermediate 81 and 6-(3- ethylureido)- 4-(4- ([phenyl)thiazole- 2-yl)pyridine- 3-ylboronic acid (WO2009106885) |
| 24 | 7-(2-(dimethylamino)ethoxy)-1-(2- (dimethylamino)ethyl)-6-(6-(3- ethylureido)-4-(4-phenylthiazol-2- yl)pyridin-3-yl)-4-oxo-1,4- dihydroquinoline-3-carboxylic acid 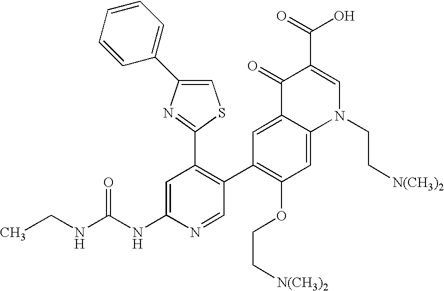 | Calcd for C₃₅H₃₉N₇O₅S [M + H]⁺: 670.25. H¹NMR (d₆-DMSO) δ 15.40 (s, 1H), 9.39 (s, 1H), 8.93 (s, 1H), 8.29 (s, 1H), 8.28 (s, 1H), 8.23 (s, 1H), 8.14 (s, 1H), 8.14 (s, 1H), 7.71-7.68 (m, 2H), 7.39-7.32 (m, 5H), 4.71-4.66 (m, 2H), 4.18-3.92 (m, 2H), 3.26-3.17 (m, 2H), 2.68-2.65 (m, 2H), 2.32-2.27 (m, 2H), 2.19 (s, 6H), 1.98 (s, 6H), 1.13 (t, 3H) | Intermediate 82 and 6-(3- ethylureido)- 4-(4- ([phenyl)thiazole- 2-yl)pyridine- 3-ylboronic acid (WO2009106885) |
| 25 | 7-(2-(dimethylamino)ethoxy)-1-(((S)-1- ethylpyrrolidin-2-yl)methyl)-6-(6-(3- ethylureido)-4-(4-(trifluoromethyl)thiazol- 2-yl)pyridin-3-yl)-4-oxo-1,4- dihydroquinoline-3-carboxylic acid 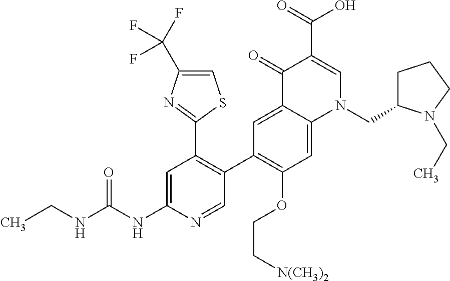 | Calcd for C₃₃H₃₈F₃N₁₇O₅S [M + H]⁺: 702.24. H¹NMR (d₆-DMSO) δ 15.39 (s, 1H), 9.42 (s, 1H), 8.90 (s, 1H), 8.50 (s, 1H), 8.28 (s, 1H), 8.26 (s, 1H), 8.25 (s, 1H), 7.60 (t, 1H), 7.39 (s, 1H), 4.67-4.63 (m, 1H), 4.4 1- 4.38 (m, 1H), 4.08-4.00 (m, 2H), 3.26-3.17 (m, 2H), 3.07- 3.05 (m, 1H), 3.02-3.00 (m, 1H), 2.28-2.12 (m, 4H), 1.98 (s, 6H), 1.71-1.68 (m, 2H), 1.55-1.50 (m, 2H), 1.23 (t, 2H), 1.12 (t, 3H), 0.97 (t, 3H) | Intermediate 81 and 6-(3- ethylureido)4-(4- (trifluoromethyl) thiazole-2- yl)pyridine- 3-ylboronic acid (WO2009106885) |
| 26 | 7-(2-(dimethylamino)ethoxy)-1-(2- (dimethylamino)ethyl)-6-(6-(3- ethylureido)-4-(4-(trifluoromethyl)thiazol- 2-yl)pyridin-3-yl)-4-oxo-1,4- dihydroquinoline-3-carboxylic acid 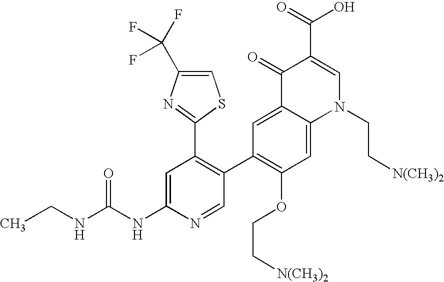 | Calcd for C₃₀H₃₄F₃N₇O₅S [M + H]⁺: 662.24. H¹NMR (d₆-DMSO) δ 9.63 (s, 1H), 9.15 (s, 1H), 8.49 (s, 1H), 8.38 (s, 1H), 8.29 (s, 1H), 8.23 (s, 1H), 7.62 (s, 1H), 7.34 (t, 1H), 5.20-5.14 (m, 2H), 4.83-4.80 (m, 2H), 3.26-3.17 (m, 2H), 3.24-3.20 (m, 2H), 2.92-2.85 (m, 2H), 2.58 (s, 6H), 1.10 (t, 3H) | Intermediate 82 and 6-(3- ethylureido)4-(4- (trifluoromethyl) thiazole-2- yl)pyridine- 3-ylboronic acid (WO2009106885) |
| 27 | 6-(6-(3-ethylureido)-4-(4- (trifluoromethyl)thiazol-2-yl)pyridin-3- yl)-7-fluoro-1-((S)-1-hydroxy-3,3- dimethylbutan-2-yl)-4-oxo-1,4- dihydroquinoline-3-carboxylic acid 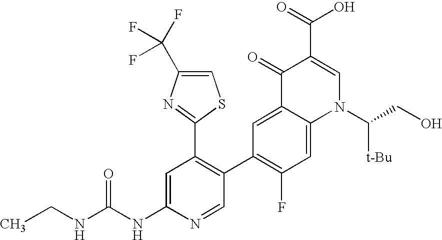 | Calcd for C₂₈H₂₇F₄N₅O₅S [M + H]⁺: 622.05. H¹NMR (d₆-DMSO) δ 9.54 (s, 1H), 8.87 (s, 1H), 8.56 (s, 1H), 8.45 (d, 1H), 8.40 (s, 1H), 8.35 (s, 1H), 8.22 (s, 1H), 7.54 (t, 1H), 5.10-4.99 (m, 1H), 4.16-4.04 (m, 2H), 3.26-3.17 (m, 2H), 1.11 (t, 3H), 0.97 (s, 9H) | Intermediate 76 and 6-(3- ethylureido)- 4-(4- (trifluoromethyl) thiazole-2- yl)pyridine- 3-ylboronic acid (WO2009106885) |

### Example 28

### 6-(6-(3-ethylureido)-4-(4-(trifluoromethyl)thiazol-2-yl)pyridin-3-yl)-1-((S)-1-hydroxy-3-methylbutan-2-yl)-7-methoxy-4-oxo-1,4-dihydroquinoline-3-carboxylic acid

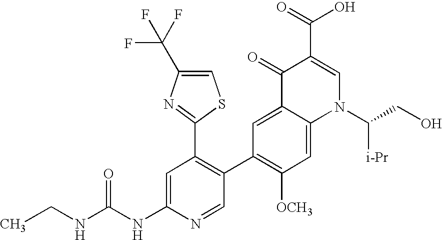

6-(6-(3-ethylureido)-4-(4-(trifluoromethyl)thiazol-2-yl)pyridin-3-yl)-7-fluoro-1-((S)-1-hydroxy-3-methylbutan-2-yl)-4-oxo-1,4-dihydroquinoline-3-carboxylic acid (Example 21) in a solution of 0.5 M sodium methoxide in methanol (4.7 mL, 2.33 mmol, 10 equiv.) was stirred at 65° C. for 8 h. The reaction mixture was cooled to room temperature and concentrated under reduced pressure. The residue was re-suspended in water, and 1 N HCl was added until pH 3-4 was reached. The aqueous layer was extracted with dichloromethane (3×10 mL), and the organics were concentrated. The compound was purified (HPLC) and concentrated to provide 6-(6-(3-ethylureido)-4-(4-(trifluoromethyl)thiazol-2-yl)pyridin-3-yl)-1-((S)-1-hydroxy-3-methylbutan-2-yl)-7-methoxy-4-oxo-1,4-dihydroquinoline-3-carboxylic acid as a solid (52.6 mg, 36%).

Calcd for C₂₉H₃₀F₃N₅O₆S [M+H]⁺: 634.18.

NMR (d₆-DMSO) δ 15.35 (s, 1H), 9.47 (s, 1H), 8.89 (s, 1H), 8.51 (s, 1H), 8.29 (s, 1H), 8.28 (s, 1H), 8.21 (d, 1H), 7.64 (t, 1H), 7.48 (s, 1H), 5.33-5.18 (m, 2H), 3.95-3.74 (m, 2H), 3.66-3.61 (m, 2H), 3.26-3.17 (m, 2H), 2.05-1.73 (m, 2H), 1.56-1.36 (m, 1H), 1.11 (t, 3H), 0.93 (d, 3H), 0.89 (d, 3H).

### Example 29

### 1-[2-(Dimethylamino)ethyl]-6-{6-[(ethylcarbamoyl)amino]-4-[4-(trifluoromethyl)-1,3-thiazol-2-yl]pyridin-3-yl}-N,N-dimethyl-4-oxo-1,4-dihydroquinoline-3-carboxamide

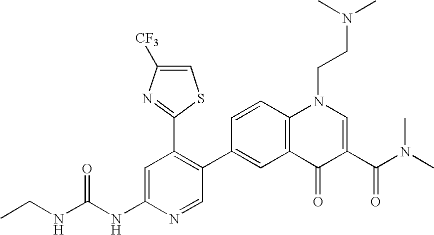

In a round bottomed flask 1-[2-(dimethylamino)ethyl]-6-iodo-N,N-dimethyl-4-oxo-1,4-dihydroquinoline-3-carboxamide (Intermediate 95) (0.12 g, 0.290 mmol) and Intermediate 12 (0.128 g, 0.298 mmol) and cesium carbonate (0.188 g, 0.581 mmol) were suspended in 1,4-dioxane:water (5:1) (12 mL) and argon was purged for 30 min. To the above reaction mixture was added tetrakis(triphenylphosphine) palladium (40.2 mg, 0.0348 mmol) under argon atmosphere and the resulting reaction mixture was heated at 80° C. for 1 h. After the completion of the reaction, the reaction mixture was filtered through a bed of Celite. The filtrate was concentrated to dryness and purified by silica gel flash column chromatography (0-10% methanol in dichloromethane) to obtain 0.07 g of the title compound 1-[2-(dimethylamino)ethyl]-6-{6-[(ethylcarbamoyl)amino]-4-[4-(trifluoromethyl)-1,3-thiazol-2-yl]pyridin-3-yl}-N,N-dimethyl-4-oxo-1,4-dihydroquinoline-3-carboxamide in a 40% yield.

LC-MS: m/z 601(M+H)

¹H NMR (400 MHz, DMSO-d₆): δ 1.12 (t, 3H), 2.21 (s, 6H), 2.62 (s, 3H), 2.84 (s, 3H), 2.92 (m, 2H), 3.21 (q, 2H), 4.41 (d, 2H), 7.62 (d, 1H), 7.82 (d, 1H), 8.18 (s, 1H), 8.21 (m, 2H), 8.38 (s, 1H), 8.48 (s, 1H), 9.42 (s, 1H).

### Example 30-31

The following Examples were prepared according to the procedure described for Example 29 from the starting material indicated in the table.

| Ex | Compound | Data | SM |
|---|---|---|---|
| 30 | 6-{6-[(Ethylcarbamoyl)amino]-4-[4- (trifluoromethyl)-1,3-thiazol-2-yl]pyridin- 3-yl}-N,N-dimethyl-1-[2-(4- methylpiperazin-1-yl)ethyl]-4-oxo-1,4- dihydroquinoline-3-carboxamide 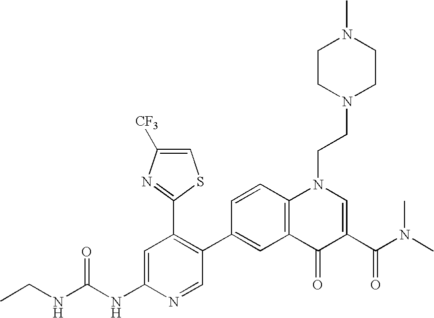 | ¹H NMR (400 MHz, DMSO- d₆): δ 1.1 (t, 3H), 2.20 (s, 6H), 2.61-2.67 (m, 2H), 2.88 (s, 3H), 2.96 (s, 3H), 3.17- 3.22 (m, 2H), 4.41-4.44 (t, 2H), 7.63-7.67 (d, 1H ), 7.83- 7.85 (d, 1H), 8.13-8.14 (d, 1H), 8.18-8.22 (d, 2H), 8.34 (s, 1H), 8.48 (s, 1H), 9.44 (s, 1H). LC-MS: m/z 656 (M + H) | Intermediate 12 and 96 |
| 31 | 1-(1,4-diazabicyclo[2.2.2]oct-2-ylmethyl)- 6-{6-[(ethylcarbamoyl)amino]-4-[4- (trifluoromethyl)-1,3-thiazol-2-yl]pyridin- 3-yl}-N,N-dimethyl-4-oxo-1,4- dihydroquinoline-3-carboxamide 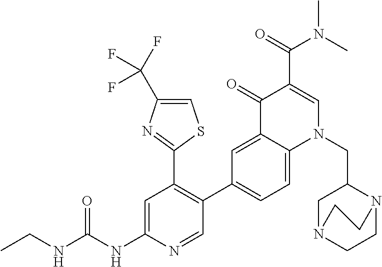 | ¹H NMR (400 MHz, DMSO- d₆₎ δ 1.09 (t, 3H), 2.28 (t, 1H) 2.51 (m, 5H), 2.87 (s, 3H), 3.19 (m, 7H), 4.36 (m, 2H), 7.66 (m, 2H), 7.90 (d, 1H), 8.11 (d, 2H), 8.22 (s, 1H), 8.33 (s, 1H), 8.84 (s, 1H), 9.45 (s, 1H) LC-MS: m/z 655.4 (M + H) | Intermediate 12 and 99 |

### Example 32

### 6-{6-[(Ethylcarbamoyl)amino]-4-[4-(trifluoromethyl)-1,3-thiazol-2-yl]pyridin-3-yl}-N,N-dimethyl-4-oxo-1-[(3R)-piperidin-3-ylmethyl]-1,4-dihydroquinoline-3-carboxamide

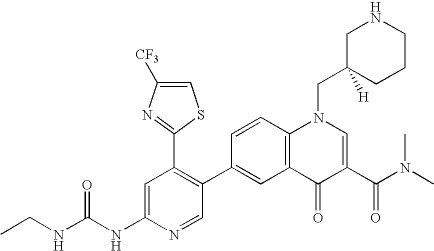

To a stirred solution of tert-butyl (3S)-3-{[3-(dimethylcarbamoyl)-6-{6-[(ethylcarbamoyl)amino]-4-[4-(trifluoromethyl)-1,3-thiazol-2-yl]pyridin-3-yl}-4-oxoquinolin-1(4H)-yl]methyl}piperidine-1-carboxylate (Intermediate 98) (0.28 g, 0.38 mmol) in dichloromethane (40 mL), was added trifluoroacetic acid (0.3 mL, 3.84 mmol) at 0° C. The reaction mixture was stirred at room temperature for 16 h. The reaction mixture was quenched with sodium bicarbonate solution (30 mL). The layers were separated and organic layer was dried and concentrated under reduced pressure. The crude product was triturated with diethyl ether (30 mL) to obtain 0.075 g of the title compound 6-{6-[(ethylcarbamoyl)amino]-4-[4-(trifluoromethyl)-1,3-thiazol-2-yl]pyridin-3-yl}-N,N-dimethyl-4-oxo-1-[(3R)-piperidin-3-ylmethyl]-1,4-dihydroquinoline-3-carboxamide in a 31% yield.

LC-MS: m/z 628(M+H)

¹H NMR (400 MHz, CD₃OD): δ 1.22 (m, 6H), 1.71 (m, 2H), 2.18 (s, 1H), 2.42 (m, 2H), 3.02 (m, 8H), 3.21 (q, 2H), 3.38 (m, 2H), 4.25 (m, 2H), 7.65 (d, 1H), 7.85 (d, 2H), 8.19 (s, 2H), 8.39 (s, 2H).

### Example 33-34

The following Examples were prepared according to the procedure described for Example 32 from the starting material indicated in the table.

| Ex | Compound | Data | SM |
|---|---|---|---|
| 33 | 6-{6-[(Ethylcarbamoyl)amino]-4-[4- | ¹H NMR (400 MHz, CD₃0D): | Intermediate |
| | (trifluoromethyl)-1,3-thiazol-2-yl]pyridin- | δ 1.21 (m, 6H), 1.72 (m, 2H), | 97 and HCl |
| | 3-yl}-N,N-dimethyl-4-oxo-1-[(3S)- | 2.19 (s, 1H) 2.42 (m, 2H), | |
| | piperidin-3-ylmethyl]-1,4- | 3.02 (m, 8H), 3.21 (q, 2H), | |
| | dihydroquinoline-3-carboxamide | 3.34 (m, 2H), 4.24 (m, 2H), | |
| | 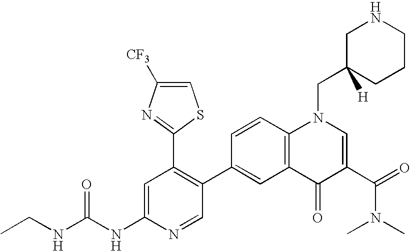 | 7.70 (d, 1H), 7.90 (d, 2H), 8.19 (s, 2H), 8.39 (s, 2H). LC-MS: m/z 414 (M + H) | |
| 34 | 1-(2-Aminoethyl)-6-{6- | ¹H NMR (400 MHz, DMSO- | Intermediate |
| | [(ethylcarbamoyl)amino]-4-[4- | d₆): 1.09-1.13 (t, 3H), 3.20- | 107 |
| | (trifluoromethyl)-1,3-thiazol-2-yl]pyridin- | 3.27 (m, 4H), 4.74 (m, 2H), | |
| | 3-yl}-4-oxo-1,4-dihydroquinoline-3- | 7.61 (s, 1H), 7.87-7.89 (d, | |
| | carboxylic acid | 1H), 8.09-8.11 (d, 1H), 8.22 | |
| | 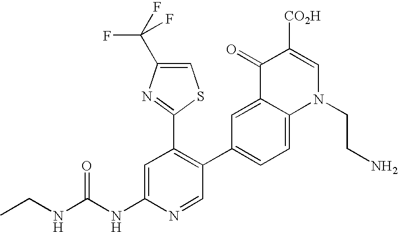 | (s, 1H), 8.33-8.36 (m, 2H), 8.55 (s, 1H), 9.01 (s, 1H), 9.50 (s, 1H) LC-MS: m/z 547.43 (M + H). | |

### Example 35

### 6-{6-[(ethylcarbamoyl)amino]-4-[4-(trifluoromethyl)-1,3-thiazol-2-yl]pyridin-3-yl}-1-[(1R,5S,6s)-3-methyl-3-azabicyclo[3.1.0]hex-6-yl]-4-oxo-1,4-dihydroquinoline-3-carboxylic acid

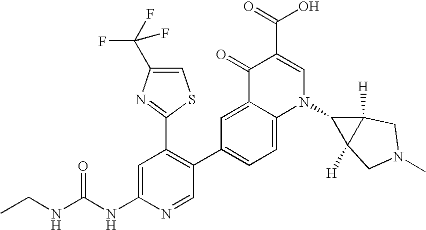

To a stirred solution of ethyl 6-{6-[(ethylcarbamoyl)amino]-4-[4-(trifluoromethyl)-1,3-thiazol-2-yl]pyridin-3-yl}-1-[(1R,5S,6s)-3-methyl-3-azabicyclo[3.1.0]hex-6-yl]-4-oxo-1,4-dihydro quinoline-3-carboxylate (Intermediate 106) (250 mg, 0.39 mmol) in ethanol (30 mL) was added 10% potassium hydroxide (2.5 mL) at room temperature. The reaction mixture was heated to 70° C. for 2 h. After completion of the reaction, the reaction mixture was cooled to room temperature. The organic solvent was concentrated under reduced pressure. The residue was diluted with water and acidified with 2N HCl (pH ˜5-6) to obtain solid, which was filtered, washed with water and dried to afford 220 mg of the title compound 6-{6-[(ethylcarbamoyl)amino]-4-[4-(trifluoromethyl)-1,3-thiazol-2-yl]pyridin-3-yl}-1-[(1R,5S,6s)-3-methyl-3-azabicyclo[3.1.0]hex-6-yl]-4-oxo-1,4-dihydroquinoline-3-carboxylic acid (Example 35) as off-white solid in a 86% yield.

¹H NMR (400 MHz, DMSO-d₆): δ 1.11 (m, 3H), 2.81 (m, 5H), 3.21 (m, 2H), 3.50 (m, 2H), 4.04 (m, 2H), 4.49 (m, 1H), 7.70 (s, 1H), 7.92 (d, 1H), 8.10 (m, 1H), 8.29 (m, 3H), 8.52 (s, 1H), 8.94 (s, 1H), 9.54 (s, 1H), 11.52 (brs, 1H).

LC-MS: m/z 599.39 (M+H)

### Example 36

### (R)-6-(6-(3-ethylureido)-4-(4-(trifluoromethyl)thiazol-2-yl)pyridin-3-yl)-1-((1-(2-(4-methylpiperazin-1-yl)ethyl)pyrrolidin-3-yl)methyl)-4-oxo-1,4-dihydroquinoline-3-carboxylic acid

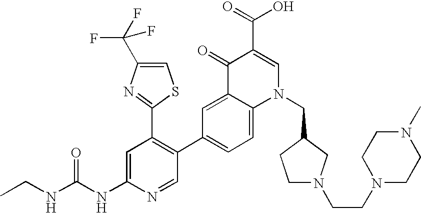

Compound (R)-ethyl 6-(6-(3-ethylureido)-4-(4-(trifluoromethyl)thiazol-2-yl)pyridin-3-yl)-1-((1-(2-(4-methylpiperazin-1-yl)ethyl)pyrrolidin-3-yl)methyl)-4-oxo-1,4-dihydroquinoline-3-carboxylate (Intermediate 121) (0.459 mmol) was dissolved in methanol (10 mL) and tetrahydrofuran (10 mL). To this solution was added 24 wt % sodium hydroxide (1 mL) and the reaction was stirred at room temperature until complete. The solution was then concentrated to remove the organic solvents. Water (10 mL) was added and the aqueous layer was acidified to pH 2-3 with 1N hydrochloric acid. The solution was concentrated, triturated with water, and filtered. The solid was re-dissolved in methanol and concentrated to give light yellow solid product. The filtrates, which contained impure product, were purified by prep HPLC.

MS (ESP): 713.1 (MH⁺) for C₃₄H₃₉F₃N₈O₄S. ¹H NMR (300 MHz, CD₃OD): δ ppm 1.21 (t, 3H), 1.2-1.4 (bm, 3H), 2.0-2.2 (br, 1H), 2.2-2.4 (br, 1H), 2.4-2.6 (bm, 2H), 2.92 (m, 2H), 2.92 (s, 3H), 3.1-3.8 (12H), 4.78 (br, 2H), 7.84 (dd, 1H), 7.89 (s, 1H), 8.12 (b, 1H), 8.22 (s, 1H), 8.36 (s, 1H), 8.44 (d, 1H), 9.10 (s, 1H)

¹⁹F NMR (300 MHz, CD₃OD): δ ppm-65.88

### Example 37

The following Examples were prepared according to the procedure described for Example 36 from the starting material indicated in the table.

| Ex | Compound | Data | SM |
|---|---|---|---|
| 37 | (S)-6-(6-(3-ethylureido)-4-(4- | MS (ESP): 713.4 (MH⁺) for | Intermediate |
| | (trifluoromethyl)thiazol-2-yl)pyridin-3- | C₃₄H₃₉F₃N₈O₄S | 122 and |
| | yl)-1-((1-(2-(4-methylpiperazin-1- | ¹H NMR (300 MHz, CD₃OD): δ | NaOH |
| | yl)ethyl)pyrrolidin-3-yl)methyl)-4-oxo- | ppm 1.23 (t, 3H), 1.85 (br, 1H), | |
| | 1,4-dihydroquinoline-3-carboxylic acid | 2.1-2.3 (br, 2H), 2.6-2.85 (bm, | |
| | 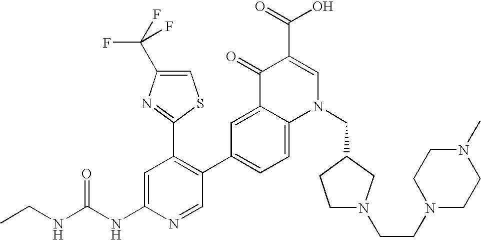 | 6H), 2.7 (s, 3H), 2.9-3.2 (10H), 3.35 (q, 2H), 4.5-4.7 (br, 2H), 7.81 (d, 1H), 7.87 (s, 1H), 8.04 (d, 1H), 8.20 (s, 1H), 8.37 (s, 1H), 8.44 (s, 1H), 9.01 (s, 1H) ¹⁹F NMR (300 MHz, CD₃OD): δ ppm −65.91 | |

### Example 38

### Acetoxymethyl 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylate

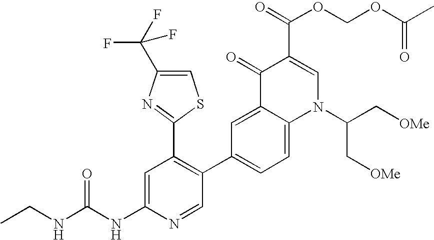

To a solution of 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylic acid (Intermediate 43) (0.33 mmol) in dimethyl formamide (1-2 mL) was added potassium carbonate (0.66 mmol) and the solution stirred for 30 min. Then bromomethyl acetate (0.66 mmol) was added and the solution stirred overnight. The reaction was diluted with ethyl acetate and sodium bicarbonate solution. The layers were separated; the organic layer was dried over sodium sulfate, and evaporated. The crude product was purified by Analogix (heptane/ethyl acetate).

MS (ESP): 678.3 (M+H⁺) for C₃₀H₃₀F₃N₅O₈S

¹H NMR (300 MHz, CD₃OD): δ ppm 9.02 (s, 1H), 8.40 (d, 1H), 8.35 (s br, 1H), 8.15 (s, 1H), 7.98 (m, 1H), 7.88 (s, 1H), 7.70 (dd, 1H), 5.94 (s, 2H), 5.30 (m, 1H), 3.91 (m, 4H), 3.36 (m, 8H), 2.14 (s. 3H), 1.23 (t, 3H).

### Example 39-44

The following Examples were prepared according to the procedure described for Example 38 from the starting material indicated in the table.

| Ex | Compound | Data | SM |
|---|---|---|---|
| 39 | Pivaloyloxymethyl 1-(1,3- | MS (ESP): 720.3 | Intermediate |
| | dimethoxypropan-2-yl)-6-(6-(3- | (M + H⁺) for | 43 and |
| | ethylureido)-4-(4- | C₃₃H₃₆F₃N₅O₈S | iodomethyl |
| | (trifluoromethyl)thiazol-2-yl)pyridin-3- | ¹H NMR (300 MHz, | pivalate |
| | yl)-4-oxo-1,4-dihydroquinoline-3- | dmso-d₆): δ ppm 9.45 (s, | |
| | carboxylate | 1H), 8.73 (s, 1H), 8.51 | |
| | 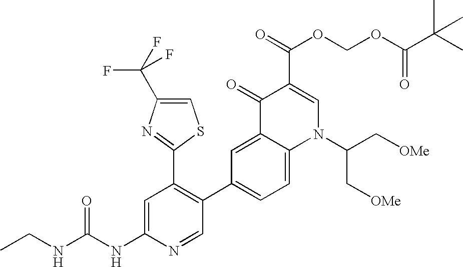 | (s, 1H), 8.33 (s, 1H), 8.21 (s, 1H), 8.18 (d, 1H), 8.08 (d, 2H), 7.71 (m, 1H), 7.64 (m, 1H), 5.89 (s, 2H), 5.40 (m, 1H), 3.80 (m, 4H), 3.26 (s, 6H), 3.20 (q, 2H), 1.17 (s. 9H), 1.11 (t, 3H) | |
| 40 | (5-methyl-2-oxo-1,3-dioxo1-4-yl)methyl | MS (ESP): 718.2 | Intermediate |
| | 1-(1,3-dimethoxypropan-2-yl)-6-(6-(3- | (M + H⁺) for | 43 and 4- |
| | ethylureido)-4-(4- | C₃₂H₃₀F₃N₅O₉S | (chloromethyl)- |
| | (trifluoromethyl)thiazol-2-yl)pyridin-3- | ¹H NMR (300 MHz, | 5-methyl-1,3- |
| | yl)-4-oxo-1,4-dihydroquinoline-3- | dmso-d₆): δ ppm 9.46 (s, | dioxol-2-one |
| | carboxylate | 1H), 8.77 (s, 1H), 8.51 | |
| | 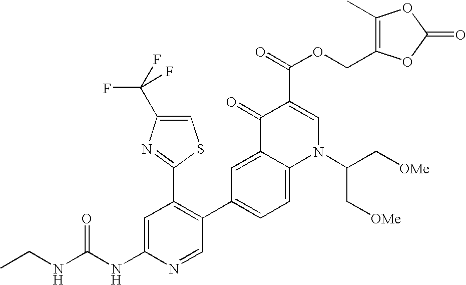 | (d, 1H), 8.33 (d, 1H), 8.22 (d, 1H), 8.17 (d, 1H), 8.07 (d, 1H), 7.70 (dd, 1H), 7.65 (m, 1H), 5.37 (m, 1H), 5.14 (s, 2H), 3.88 (m, 4H), 3.27 (s, 6H), 3.21 (q, 2H), 2.23 (s, 3H), 1.11 (t, 3H) | |
| 41 | 2-acetoxyethyl 1-(1,3-dimethoxypropan- | MS (ESP): 692.1 | Intermediate |
| | 2-yl)-6-(6-(3-ethylureido)-4-(4- | (M + H⁺) for | 43, thionyl |
| | (trifluoromethyl)thiazol-2-yl)pyridin-3- | C₃₁H₃₂F₃N₅O₈S | chloride, |
| | yl)-4-oxo-1,4-dihydroquinoline-3- | ¹H NMR (300 MHz, | bromoethanol |
| | carboxylate | CD₃OD): δ ppm 8.97 (s, | and acetic |
| | 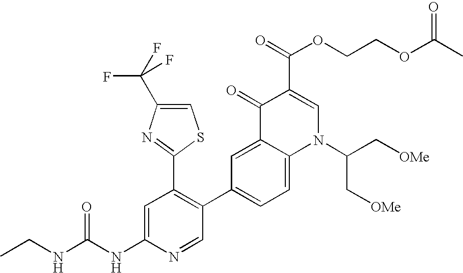 | 1H), 8.41 (s, 1H), 8.40 (s br, 1H), 8.15 (s, 1H), 7.99 (m, 1H), 7.88 (s, 1H), 7.67 (dd, 1H), 5.31 (m, 1H), 4.46 (m, 4H), 3.91 (m, 4H), 3.36 (m, 8H), 2.08 (s. 3H), 1.23 (t, 3H) | acid |
| 42 | 2-(propionyloxy)ethyl 1-(1,3- | MS (ESP): 706.2 | Intermediate |
| | dimethoxypropan-2-yl)-6-(6-(3- | (M + H⁺) for | 43, thionyl |
| | ethylureido)-4-(4- | C₃₂H₃₄F₃N₅O₈S | chloride, |
| | (trifluoromethyl)thiazol-2-yl)pyridin-3- | ¹H NMR (300 MHz, | bromoethanol |
| | yl)-4-oxo-1,4-dihydroquinoline-3- | dmso-d₆): δ ppm 9.47 (s, | and |
| | carboxylate | 1H), 8.70 (s, 1H), 8.51 | propionic |
| | 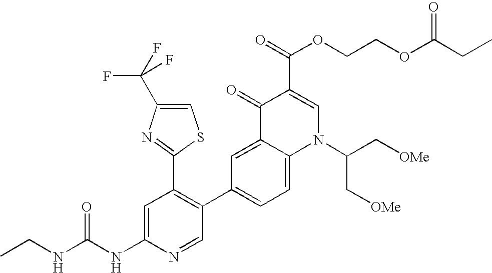 | (d, 1H), 8.33 (d, 1H), 8.22 (s, 1H), 8.17 (d, 1H), 8.07 (d, 2H), 7.69 (dd, 1H), 7.64 (m, 1H), 5.37 (m, 1H), 4.39 (m, 2H), 4.35 (m, 2H), 3.80 (m, 4H), 3.27 (s, 6H), 3.21 (q, 2H), 2.34 (q, 2H), 1.11 (t, 3H), 1.03 (t, 3H) | acid |
| 43 | 2-(isobutyryloxy)ethyl 1-(1,3- | MS (ESP): 720.0 (M + H⁺) for | Intermediate |
| | dimethoxypropan-2-yl)-6-(6-(3- | C₃₃H₃₆F₃N₅O₈S | 43, thionyl |
| | ethylureido)-4-(4-(trifluoromethyl)thiazol- | ¹H NMR (300 MHz, dmso- | chloride, |
| | 2-yl)pyridin-3-yl)-4-oxo-1,4- | d₆): δ ppm 9.47 (s, 1H), 8.70 | bromoethanol |
| | dihydroquinoline-3-carboxylate | (s, 1H), 8.51 (d, 1H), 8.33 (d, | and |
| | 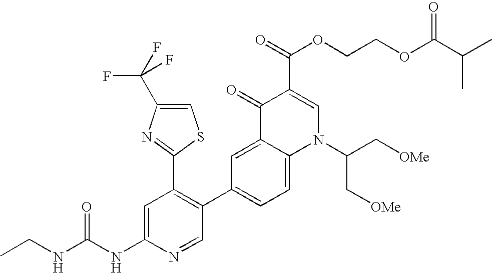 | 1H), 8.21 (d, 1H), 8.17 (d, 1H), 8.07 (d, 2H), 7.69 (dd, 1H), 7.64 (m, 1H), 5.37 (m, 1H), 4.39 (m, 2H), 4.34 (m, 2H), 3.89 (m, 4H), 3.27 (s, 6H), 3.20 (q, 2H), 2.56 (m, 1H), 1.10 (m, 9H) | isobutyric acid |
| 44 | 2-(pivaloyloxy)ethyl 1-(1,3- | MS (ESP): 734.4 (M + H⁺) for | Intermediate |
| | dimethoxypropan-2-yl)-6-(6-(3- | C₃₄H₃₈F₃N₅O₈S | 43, thionyl |
| | ethylureido)-4-(4-(trifluoromethyl)thiazol- | ¹H NMR (300 MHz, dmso- | chloride, |
| | 2-yl)pyridin-3-yl)-4-oxo-1,4- | d₆): δ ppm 9.46 (s, 1H), 8.70 | bromoethanol |
| | dihydroquinoline-3-carboxylate | (s, 1H), 8.51 (d, 1H), 8.33 (d, | and pivalic |
| | 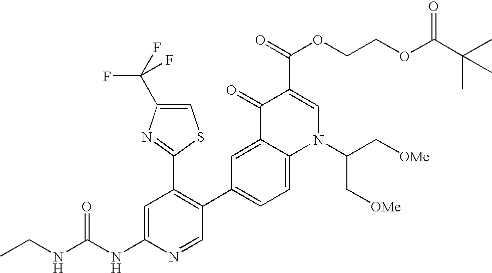 | 1H), 8.21 (d, 1H), 8.17 (d, 1H), 8.07 (d, 2H), 7.69 (dd, 1H), 7.64 (m, 1H), 5.37 (m, 1H), 4.39 (m, 2H), 4.34 (m, 2H), 3.89 (m, 4H), 3.26 (s, 6H), 3.21 (q, 2H), 1.14 (s, 9H), 1.11 (t, 3H) | acid |

### Examples 45-52

The following examples were prepared by the procedure described in Example 29 from the indicated starting material.

| Ex | Compound | Data | SM |
|---|---|---|---|
| 45 | (R)-7-(2-(diethylamino)ethoxy)-1- | Calcd for C₄₀H₄₇N₇O₅S [M + H]⁺: | W02009106885 |
| | ((1-ethylpyrrolidin-2-yl)methyl)-6- | 738.4. | & |
| | (6-(3-ethylureido)-4-(4- | H¹NMR (d₆-DMSO) δ 15.26 (s, | Intermediate |
| | phenylthiazol-2-yl)pyridin-3-yl)-4- | 1H), 9.41 (s, 1H), 8.38 (s, 1H), | 123 |
| | oxo-1,4-dihydroquinoline-3- | 8.33 (s, 1H), 8.26 (s, 1H), 8.17 (s, | |
| | carboxylic acid | 1H), 7.79-7.75 (m, 2H), 7.61 (t, | |
| | 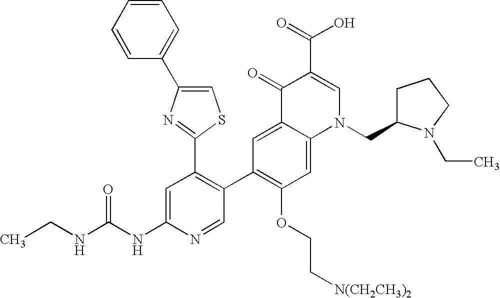 | 1H), 7.47-7.33 (m, 5H), 4.73 (m, 1H), 4.51-4.28 (m, 4H), 3.26-3.21 (m, 4H), 2.95-2.9 (m, 4H), 2.27- 1.98 (m, 2H), 1.95-1.90 (m, 2H), 1.75-1.72 (m, 2H), 1.56-1.52 (m, 2H), 1.12 (t, 3H), 0.95 (t, 6H), 0.77 (t, 3H). | |
| 46 | 7-(2-(diethylamino)ethoxy)-1-(2- | Calcd for C₃₇H₄₃N₇O₅S [M + H]⁺: | W02009106885 |
| | (dimethylamino)ethyl)-6-(6-(3- | 698.31. | & |
| | ethylureido)-4-(4-phenylthiazol-2- | H¹NMR (d₆-DMSO) δ 15.33 (s, | Intermediate |
| | yl)pyridin-3-yl)-4-oxo-1,4- | 1H), 9.4 (s, 1H), 8.95 (s, 1H), 8.33 | 82 |
| | dihydroquinoline-3-carboxylic acid | (s, 1H), 8.3 (s, 1H), 8.24 (s, 1H), | |
| | 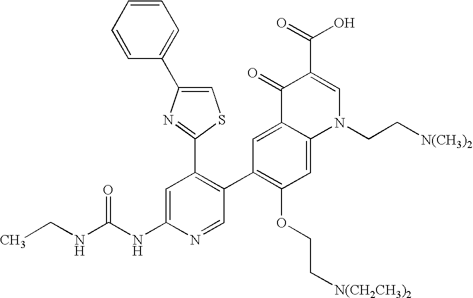 | 8.15 (s, 1H), 8.13 (s, 1H), 7.72 (t, 1H), 7.72-7.68 (m, 2H), 7.38-7.29 (m, 3H), 4.73 (m, 2H), 4.35-4.3 (m, 2H), 4.08-3.98 (m, 2H), 3.28-3.19 (m, 4H), 2.94-2.81 (m, 2H), 2.73- 2.68 (m, 2H), 2.24 (s, 6H), 1.12 (t, 3H), 0.83 (t, 6H). | |
| 47 | 7-(2-(dimethylamino)ethoxy)-1- | Calcd for C₂₈H₂₉F₃N₆O₅S [M + | Intermediate |
| | ethyl-6-(6-(3-ethylureido)-4-(4- | H]⁺: 619.24. | 12 & |
| | (trifluoromethyl)thiazol-2- | H¹NMR (d₆-DMSO) δ 10.73 (s, | Intermediate |
| | yl)pyridin-3-yl)-4-oxo-1,4- | 1H), 9.48 (s, 1H), 9.06 (s, 1H), | 124 |
| | dihydroquinoline-3-carboxylic acid | 8.47 (s, 1H), 8.33 (s, 1H), 8.29 (s, | |
| | 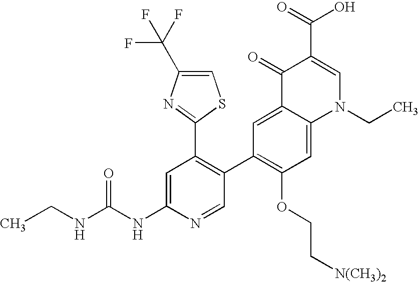 | 1H), 8.26 (s, 1H), 7.62 (t, 1H), 7.41 (s, 1H), 4.73-4.62 (m, 3H), 3.65- 3.61 (m, 2H), 3.26-3.18 (m, 4H), 2.55 (s, 6H), 1.46 (t, 3H), 1.11 (t, 3H). | |
| 48 | 1-(2-(dimethylamino)ethyl)-6-(6-(3- | Calcd for C₃₇H₄₁N₇O₆S [M + H]⁺: | Intermediate |
| | ethylureido)-4-(4-phenylthiazol-2- | 712.3. | 127 & |
| | yl)pyridin-3-yl)-7-(2- | H¹NMR (d₆-DMSO) δ 15.37 (s, | W020091068852 |
| | morpholinoethoxy)-4-oxo-1,4- | 1H), 9.4 (s, 1H), 8.96 (d, 1H), 8.54 | |
| | dihydroquinoline-3-carboxylic acid | (s, 1H), 8.31 (s, 1H), 8.3 (s, 1H), | |
| | 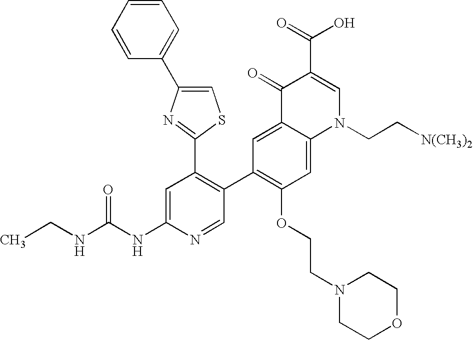 | 8.21 (s, 1H), 8.13 (s, 1H), 8.06- 8.03 (m, 1H), 7.73-7.7 (m, 2H), 7.49 (s, 1H), 7.36-7.3 (m, 3H), 4.78-4.72 (m, 2H), 4.2-4.1 (m, 2H), 3.36-3.33 (m, 2H), 3.27-3.19 (m, 2H), 2.87-2.82 (m, 4H), 2.35-2.28 (m, 8H), 2.16-2.13 (m, 4H), 1.13 (t, 3H). | |
| 49 | (S)-7-(2-(diethylamino)ethoxy)-1- | Calcd for C₄₀H₄₇N₇O₅S [M + H]⁺: | Intermediate |
| | ((1-ethylpyrrolidin-2-yl)methyl)-6- | 738.3. | 81 & |
| | (6-(3-ethylureido)-4-(4- | H¹NMR (d₆-DMSO) δ 15.38 (s, | W02009106885 |
| | phenylthiazol-2-yl)pyridin-3-yl)-4- | 1H), 9.4 (s, 1H), 8.92 (s, 1H), 8.29 | |
| | oxo-1,4-dihydroquinoline-3- | (s, 1H), 8.28 (s, 1H), 8.23 (s, 1H), | |
| | carboxylic acid | 8.13 (s, 1H), 7.76-7.72 (m, 3H), | |
| | 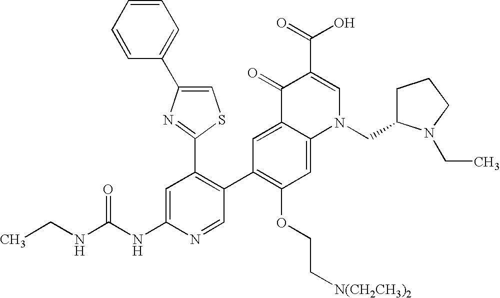 | 7.37-7.31 (m, 3H), 4.66-4.61 (m, 1H), 4.42-4.38 (m, 2H), 3.26-3.17 (m, 2H), 3.1-3.06 (m, 1H), 2.97- 2.92 (m, 1H), 2.45-2.41 (m, 2H), 2.27-2.19 (m, 4H), 2.19-2.12 (m, 2H), 1.89-1.85 (m, 2H), 1.7-1.65 (m, 2H), 1.55-1.51 (m, 2H), 1.12 (t, 3H), 0.72 (t, 3H), 0.7 (t, 6H). | |
| 50 | 6-(6-(3-ethylureido)-4-(4- | Calcd for C₂₈H₂₆F₄N₆O₅S [M + | Intermediate |
| | (trifluoromethyl)thiazol-2- | H]⁺: 635.31. | 128 & 12 |
| | yl)pyridin-3-yl)-7-fluoro-1-(2- | H¹NMR (d₆-DMSO) δ 14.94 (s, | |
| | morpholinoethyl)-4-oxo-1,4- | 1H), 9.54 (s, 1H), 8.95 (s, 1H), | |
| | dihydroquinoline-3-carboxylic acid | 8.57 (s, 1H), 8.44 (s, 1H), 8.4 (s, | |
| | 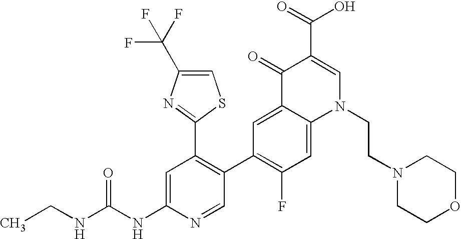 | 1H), 8.27 (s, 1H), 8.06 (d, 1H), 7.55 (s, 1H), 4.89-4.84 (m, 2H), 3.5-3.45 (m, 2H), 3.26-3.17 (m, 2H), 2.65-2.61 (m, 2H), 2.45-2.41 (m, 4H), 1.11 (t, 3H). | |
| 51 | 1-(2-(dimethylamino)propyl)-6-(6- | Calcd for C₂₇H₂₆F₄N₆O₄S [M + | Intermediate |
| | (3-ethylureido)-4-(4- | H]⁺: 607.29. | 129 & 12 |
| | (trifluoromethyl)thiazol-2- | H¹NMR (d₆-DMSO) δ 15.0 (s, | |
| | yl)pyridin-3-yl)-7-fluoro-4-oxo-1,4- | 1H), 9.55 (s, 1H), 8.85 (s, 1H), | |
| | dihydroquinoline-3-carboxylic acid | 8.59 (s, 1H), 8.45 (s, 1H), 8.41 (s, | |
| | 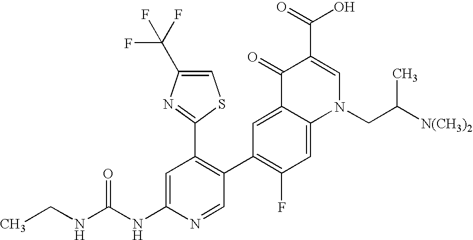 | 1H), 8.27 (s, 1H), 8.05 (d, 1H), 7.54 (t, 1H), 4.56-4.42 (m, 1H), 3.26-3.17 (m, 2H), 2.96-2.76 (m, 2H), 2.13 (s, 6H), 1.11 (t, 3H). 0.99 (d, 3H). | |
| 52 | (S)-6-(6-(3-ethylureido)-4-(4- | Calcd for C₂₈H₂₇F₄N₅O₅S [M + | Intermediate |
| | (trifluoromethyl)thiazol-2- | H]⁺: 622.06. | 130 & 12 |
| | yl)pyridin-3-yl)-7-fluoro-1-(1- | H¹NMR (d₆-DMSO) δ 14.96 (s, | |
| | hydroxy-4-methylpentan-2-yl)-4- | 1H), 9.53 (s, 1H), 8.89 (s, 1H), | |
| | oxo-1,4-dihydroquinoline-3- | 8.58 (s, 1H), 8.47 (d, 1H), 8.41 (s, | |
| | carboxylic acid | 1H), 8.28 (s, 1H), 8.26 (s, 1H), | |
| | 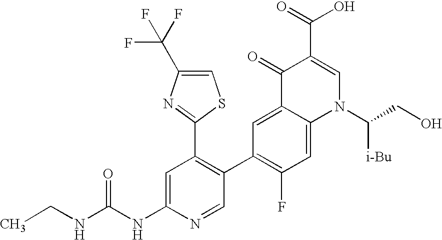 | 7.54 (t, 1H), 5.18-5.08 (m, 1H), 3.82-3.69 (m, 2H), 3.26-3.17 (m, 2H), 1.99-1.91 (m, 1H), 1.85-1.82 (m, 1H), 1.78-1.42 (m, 1H), 1.12 (t, 3H), 0.92 (d, 3H), 0.87 (d, 3H). | |

### Example 53

### 6-(4-(4-((R)-1-amino-2-methylpropyl)thiazol-2-yl)-6-(3-ethylureido)pyridin-3-yl)-1-((S)-1-hydroxy-3,3-dimethylbutan-2-yl)-4-oxo-1,4-dihydroquinoline-3-carboxylic acid

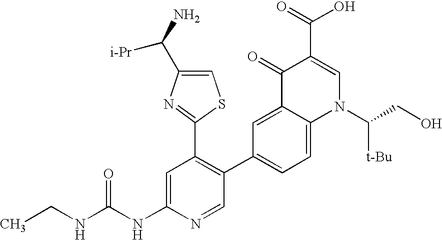

6-(4-(4-((R)-1-(benzyloxycarbonylamino)-2-methylpropyl)thiazol-2-yl)-6-(3-ethylureido)pyridin-3-yl)-1-((S)-1-hydroxy-3,3-dimethylbutan-2-yl)-4-oxo-1,4-dihydroquinoline-3-carboxylic acid (Intermediate 131) (344 mg, 0.46 mmol, 1 equiv.) and hydrogen bromide, 33% solution in acetic acid (0.925 mL, 4.64 mmol, 10 equiv.) were stirred at 23° C. for 1 h. The reaction was poured into water. 2N NaOH was added until pH 10 was reached followed by 2N HCl until pH 6 was reached and the solution was concentrated. The compound was purified via reverse phase C18 silica gel chromatography and concentrated to provide 6-(4-(4-((R)-1-amino-2-methylpropyl)thiazol-2-yl)-6-(3-ethylureido)pyridin-3-yl)-1-((S)-1-hydroxy-3,3-dimethylbutan-2-yl)-4-oxo-1,4-dihydroquinoline-3-carboxylic acid (24.9 mg, 9%).

Calcd for C₃₁H₂₈N₆O₅S [M+H]⁺: 607.26. H′NMR (d₆-DMSO) δ 9.41 (s, 1H), 8.86 (s, 1H), 8.42 (d, 1H), 8.33 (s, 1H), 8.17 (d, 1H), 8.03 (s, 1H), 7.85 (dd, 1H), 7.69 (t, 1H), 7.44 (s, 1H), 5.18-5.14 (m, 2H), 4.11-4.07 (m, 2H), 3.55 (d, 1H), 3.26-3.17 (m, 2H), 1.57-1.51 (m, 1H), 1.11 (t, 3H), 0.99 (s, 9H), 0.59 (d, 3H), 0.51 (d, 3H).

### Example 54

The following Examples were prepared according to the procedure described for Intermediate 8 from the starting material indicated in the table.

| Ex | Compound | Data | SM |
|---|---|---|---|
| 54 | 7-chloro-1-cyclopropyl-6-(6-(3- | MS (ES) (M + H)⁺: 578 for | Intermediate |
| | ethylureido)-4-(4-(trifluoromethyl)thiazol- | C₂₅H₁₉ClF₃N₅O₄S | 135 |
| | 2-yl)pyridin-3-yl)-4-oxo-1,4- | H¹NMR (d₆-DMSO) δ 14.66 | |
| | dihydroquinoline-3-carboxylic | (br. s., 1 H), 9.52 (br. s., 1 H), | |
| | acid | 8.80 (s, 1 H), 8.50 (s, 1 H), | |
| | 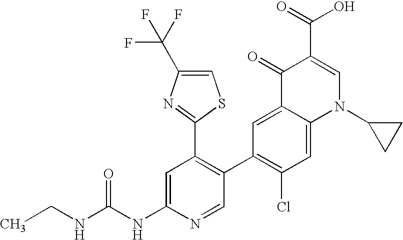 | 8.23-8.46 (m, 4 H), 7.61 (br. s., 1 H), 3.88 (br. s., 1 H), 3.16-3.25 (m, 2 H), 1.34 (d, 3 H), 1.12 (t, 4 H). | |

### Example 55

### 1-(2-(dimethylamino)ethyl)-7-(2-(dimethylamino)ethylamino)-6-(6-(3-ethylureido)-4-(4-(trifluoromethyl)thiazol-2-yl)pyridin-3-yl)-4-oxo-1,4-dihydroquinoline-3-carboxylic acid

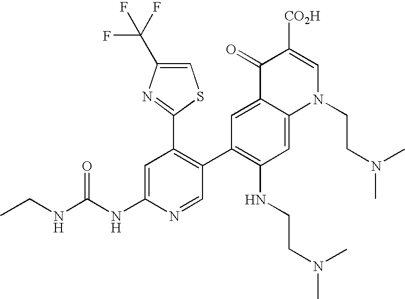

6-(3-ethylureido)-4-(4-(trifluoromethyl)thiazol-2-yl)pyridin-3-ylboronic acid (Intermediate 12) (250 mg, 0.69 mmol), 1-(2-(dimethylamino)ethyl)-7-(2-(dimethylamino)ethylamino)-6-iodo-4-oxo-1,4-dihydroquinoline-3-carboxylic acid (Intermediate 140) (328 mg, 0.69 mmol) and PdC12(PPh₃)₂ (48.7 mg, 0.07 mmol) were suspended in a 4:1 dioxane/water mixture containing potassium carbonate (165 mg, 1.04 mmol). The suspension was degassed and purged with nitrogen, and then heated to 100° C. for 2 hours. The progress of the reaction was monitored by LC/MS. The reaction was cooled to room temperature, concentrated to dryness and purified by silica gel flash column chromatography (5-50% CH₂Cl₂/MeOH). Isolation gave 70 mg of the title compound.

LC/MS (ES⁺)_{[}(M+H)⁺]: 661 for C₃₀H₃₅F₃N₈O₄S. ¹H NMR (300 MHz, d₆-DMSO): 1.12 (t, 3H), 1.99 (s, 6H), 2.16 (m, 2H), 2.23 (s, 6H), 2.68 (t, 2H), 3.18 (m, 2H), 3.22 (m, 2H), 4.54 (m, 2H), 5.72 (m, 1H), 6.67 (s, 1H), 7.89 (t, 1H), 7.98 (s, 1H), 8.18 (s, 1H), 8.38 (s, 1H), 8.43 (s, 1H), 8.70 (s, 1H).

### Example 56

### 6-(6-(3-Ethylureido)-4-(4-(trifluoromethyl)thiazol-2-yl)pyridin-3-yl)-1-(2-morpholinopropyl)-4-oxo-1,4-dihydroquinoline-3-carboxylic acid

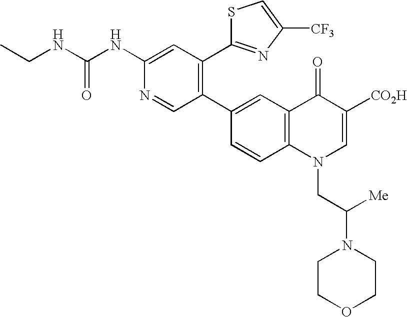

The 6-(3-ethylureido)-4-(4-(trifluoromethyl)thiazol-2-yl)pyridin-3-ylboronic acid (Intermediate 12) (0.115 g, 0.32 mmol) and ethyl 6-iodo-1-(2-morpholinopropyl)-4-oxo-1,4-dihydroquinoline-3-carboxylate, hydrochloride salt (Intermediate 141) (0.150 g, 0.32 mmol) were combined. A solution of Cs₂CO₃ (0.177 g, 0.54 mmol) in water (0.5 mL) was added. Tetrakis(triphenylphosphine)palladium(0) (0.037 g, 0.03 mmol) was added as a solution in dioxane (2 mL). The reaction mixture was heated at 100° C. for 4 h. A 2 M aq solution of LiOH (0.4 mL, 0.8 mmol) was added and the reaction mixture was heated at 100° C. for an additional 2 h. After cooling to RT, the mixture was filtered through celite, concentrated in vacuo and acidified with 1 N aq HCl. The resultant solid was collected, washed with water, and purified by reverse phase HPLC.

LC/MS (ES⁺)[(M+H)⁺]: 631 for C₂₉H₂₉F₃N₆O₅S

¹H NMR (DMSO-d₆): δ 15.10 (s, 1H); 9.46 (s, 1H); 8.85 (s, 1H); 8.52 (s, 1H); 8.38 (s, ¹H); 8.30 (m, 1H); 8.21 (s, 1H); 8.13 (d, 1H); 7.81 (dd, 1H); 7.59 (m, 1H); 4.65 (m, 1H); 4.43 (m, 1H); 3.43 (m, 4H); 3.21 (m, 2H); 2.80 (m, 3H); 2.11 (m, 2H); 1.11 (t, 3H); 1.08 (d, 3H).

### Intermediates 1-2

The compounds in the following table below were synthesized by the general procedure described below.

(S)-3-(di-tert-butoxyphosphoryloxy)propyl 1-(1-(tert-butyldimethylsilyloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylate (Intermediate 3) or (S)-2-(di-tert-butoxyphosphoryloxy)ethyl 1-(1-(tert-butyldimethylsilyloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylate (Intermediate 4) (1.40 mmol) was dissolved in THF (20 mL). A 1.0 M solution of TBAF (2.094 mL, 2.09 mmol) in THF was added. The reaction mixture was stirred at RT. After 3.5 h, the volume of the reaction mixture was reduced by half in vacuo. Another 1.5 eq of 1.0 M TBAF in THF was added. Upon completion of the reaction as determined by LCMS, saturated NH₄Cl and CH₂Cl₂ were added. The layers were separated and the organic layer was washed with water, dried over Na₂SO₄, and concentrated in vacuo to a residue which was purified by silica gel chromatography.

| Int | Compound | Data | SM |
|---|---|---|---|
| 1 | (S)-3-(di-tert- | LC/MS (ES⁺)[(M + H)⁺]: 854 | Inter- |
| | butoxyphosphoryloxy)propyl 6-(6- | for C₃₉H₅₁F₃N₅O₉PS | mediate |
| | (3-ethylureido)-4-(4- | ¹H NMR (DMSO-d₆): δ 9.44 | 3 |
| | (trifluoromethyl)thiazol-2- | (s, 1H); 8.66 (s, 1H); 8.52 (m, | |
| | yl)pyridin-3-yl)-1-(1-hydroxy-3,3- | 1H); 8.34 (s, 1H); 8.20 (m, | |
| | dimethylbutan-2-yl)-4-oxo-1,4- | 3H); 7.62 (m, 2H); 5.08 (t, | |
| | dihydroquinoline-3-carboxylate | 1H); 4.96 (t, 1H); 4.26 (t, 2H); | |
| | 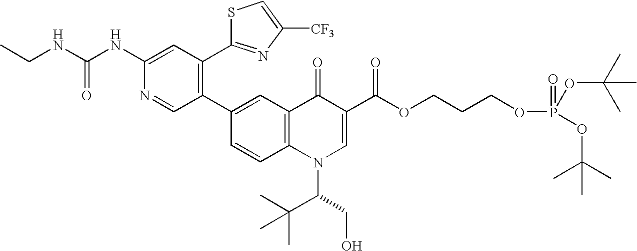 | 4.04 (m, 4H); 3.21 (m, 2H); 2.00 (m, 2H); 1.38 (br s, 18H); 1.11 (t, 3H); 0.97 (br s, 9H). | |
| 2 | (S)-2-(di-tert- | LC/MS (ES⁺)[(M + H)⁺]: 840 | Inter- |
| | butoxyphosphoryloxy)ethyl 6-(6- | for C₃₈H₄₉F₃N₅O₉PS | mediate |
| | (3-ethylureido)-4-(4- | ¹H NMR (DMSO-d₆): δ 9.44 | 4 |
| | (trifluoromethyl)thiazol-2- | (s, 1H); 8.68 (s, 1H); 8.52 (m, | |
| | yl)pyridin-3-yl)-1-(1-hydroxy-3,3- | 1H); 8.35 (s, 1H); 8.21 (m, | |
| | dimethylbutan-2-yl)-4-oxo-1,4- | 2H); 8.17 (d, 1H); 7.63 (m, | |
| | dihydroquinoline-3-carboxylate | 2H); 5.07 (t, 1H); 4.95 (t, 1H); | |
| | 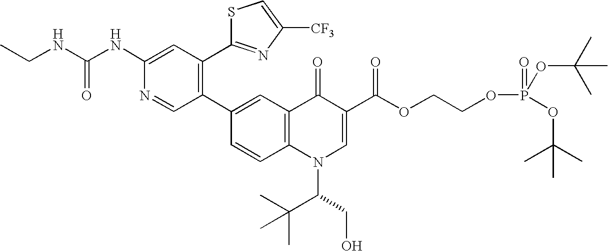 | 4.39 (m, 2H); 4.14 (m, 2H); 4.04 (m, 2H); 3.21 (m, 2H); 1.39 (m, 18H); 1.11 (t, 3H); 0.97 (br s, 9H). | |

### Intermediates 3-4

The compounds in the following table were synthesized by the general procedure described below.

(S)-3-hydroxypropyl 1-(1-(tert-butyldimethylsilyloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylate (Intermediate 5) or (S)-2-hydroxyethyl 1-(1-(tert-butyldimethylsilyloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylate (Intermediate 6, 1.40 mmol) was dissolved in DMF (7 mL). A 0.45 M solution of tetrazole (6.21 mL, 2.79 mmol) in acetonitrile was added, followed by the addition of di-tert-butyl diethylphosphoramidite (0.894 mL, 3.21 mmol). The reaction was stirred at RT for 3.5 h, then cooled to 0° C. in an ice/salt bath. A 30 wt % aqueous solution of hydrogen peroxide (0.307 mL, 3.00 mmol) was added. The cold bath was removed and the reaction mixture was stirred for 2 h. An additional 0.03 mL of 30 wt % aq hydrogen peroxide was added. After stirring at RT for 1 h, the reaction mixture was cooled to 0° C. and 0.76 M aq sodium metabisulfite (6.5 mL, 4.94 mmol) solution was added. The ice bath was removed and the reaction mixture was stirred at RT overnight. Water and EtOAc were added and the layers separated. The organic layer was washed with water. The aqueous layer was then back-extracted with EtOAc. The combined organic layers were dried over Na₂SO₄ and then concentrated in vacuo to give the product.

| Int | Compound | Data | SM |
|---|---|---|---|
| 3 | (S)-3-(di-tert- | LC/MS (ES⁺)[(M + H)⁺]: 968 | Inter- |
| | butoxyphosphoryloxy)propyl 1-(1- | for C₄₅H₆₅F₃N₅O₉PSSi | mediate |
| | (tert-butyldimethylsilyloxy)-3,3- | ¹H NMR (DMSO-d₆): δ 9.44 | 5 |
| | dimethylbutan-2-yl)-6-(6-(3- | (s, 1H); 8.64 (m, 1H); 8.54 (m, | |
| | ethylureido)-4-(4- | 1H); 8.33 (s, 1H); 8.21 (m, | |
| | (trifluoromethyl)thiazol-2- | 3H); 7.61 (m, 2H); 5.03 (m, | |
| | yl)pyridin-3-yl)-4-oxo-1,4- | 1H); 4.24 (m, 4H); 4.04 (q, | |
| | dihydroquinoline-3-carboxylate | 2H); 3.21 (m, 2H); 1.98 (m, | |
| | 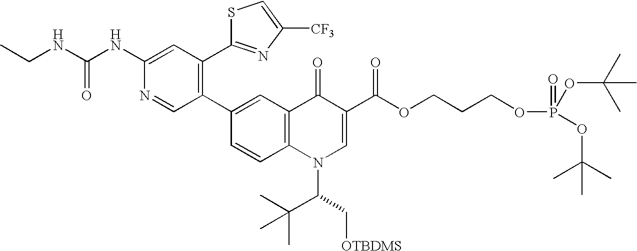 | 2H); 1.38 (s, 18H); 1.11 (t, 3H); 1.02 (br s, 9H); 0.63 (s, 9H); −0.01 (s, 3H); −0.06 (s, 3H). | |
| 4 | (S)-2-(di-tert- | LC/MS (ES⁺)[(M + H)⁺]: 954 | Inter- |
| | butoxyphosphoryloxy)ethyl 1-(1- | for C₄₄H₆₃F₃N₅O₉PSSi | mediate |
| | (tert-butyldimethylsilyloxy)-3,3- | | 6 |
| | dimethylbutan-2-yl)-6-(6-(3- | | |
| | ethylureido)-4-(4- | | |
| | (trifluoromethyl)thiazol-2- | | |
| | yl)pyridin-3-yl)-4-oxo-1,4- | | |
| | dihydroquinoline-3-carboxylate | | |
| | 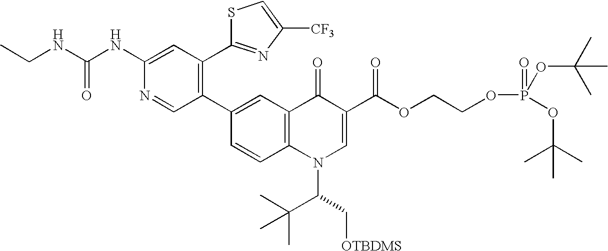 | | |

### Intermediates 5-6

The compounds in the following table were synthesized by the general procedure described below.

(S)-1-(1-(tert-butyldimethylsilyloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylic acid (Intermediate 7, 1.789 g, 2.49 mmol) was dissolved in N,N-dimethylacetamide (10 mL). Potassium carbonate (1.033 g, 7.48 mmol) was added. The reaction mixture was placed in an oil bath preheated to 70° C. After 30 min, 2 to 4 equivalents of 2-bromoethanol or 3-bromo-1-propanol was added. The reaction mixture was stirred at 70° C. overnight. Upon cooling to RT, water was added, and the resultant solid was collected and then purified by silica gel chromatography.

| Int | Compound | Data | SM |
|---|---|---|---|
| 5 | (S)-3-hydroxypropyl 1-(1-(tert- | LC/MS (ES⁺)[(M + H)⁺]: 776 for | 3-Bromo-1- |
| | butyldimethylsilyloxy)-3,3- | C₃₇H₄₈F₃N₅O₆SSi | propanol |
| | dimethylbutan-2-yl)-6-(6-(3- | | |
| | ethylureido)-4-(4- | | |
| | (trifluoromethyl)thiazol-2- | | |
| | yl)pyridin-3-yl)-4-oxo-1,4- | | |
| | dihydroquinoline-3-carboxylate | | |
| | 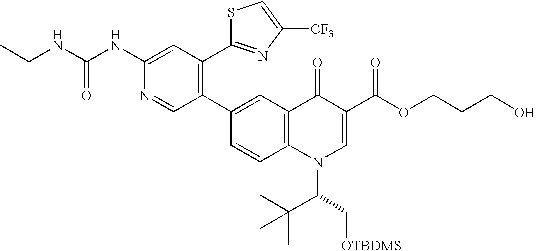 | | |
| 6 | (S)-2-hydroxyethyl 1-(1-(tert- | LC/MS (ES⁺)[(M + H)⁺]: 762 for | 2- |
| | butyldimethylsilyloxy)-3,3- | C₃₆H₄₆F₃N₅O₆SSi | Bromoethanol |
| | dimethylbutan-2-yl)-6-(6-(3- | ¹H NMR (DMSO-d₆): δ 9.44 (s, 1H); | |
| | ethylureido)-4-(4- | 8.66 (s, 1H); 8.54 (m, 1H); 8.33 (s, | |
| | (trifluoromethyl)thiazol-2- | 1H); 8.22 (m, 3H); 7.62 (m, 2H); | |
| | yl)pyridin-3-yl)-4-oxo-1,4- | 5.02 (m, 1H); 4.81 (t, 1H); 4.19 (m, | |
| | dihydroquinoline-3-carboxylate | 4H); 3.65 (q, 2H); 3.21 (m, 2H); 1.11 | |
| | 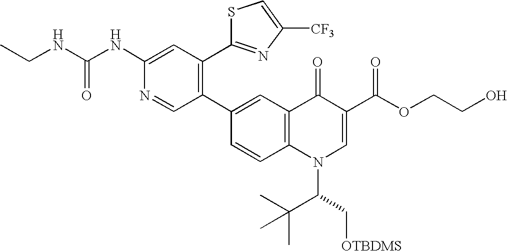 | (t, 3H); 1.01 (br s, 9H); 0.63 (br s, 9H); −0.01 (s, 3H), −0.05 (s, 3H). | |

### Intermediate 7

(S)-1-(1-(tert-butyldimethylsilyloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylic acid

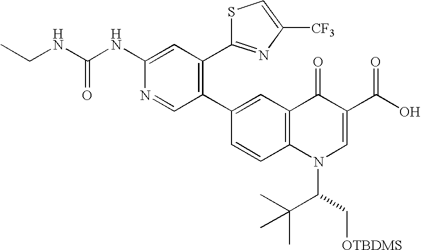

A solution of (S)-6-(6-(3-ethylureido)-4-(4-(trifluoromethyl)thiazol-2-yl)pyridin-3-yl)-1-(1-hydroxy-3,3-dimethylbutan-2-yl)-4-oxo-1,4-dihydroquinoline-3-carboxylic acid (Intermediate 8, 3.695 g, 6.12 mmol) in dichloromethane (14 mL) was cooled to 0° C. Triethylamine (2.389 ml, 17.14 mmol) was added, followed by the dropwise addition of tert-butyldimethylsilyl trifluoromethane-sulfonate (1.687 ml, 7.35 mmol). The reaction mixture was stirred at 0° C. for 2 h and then allowed to warm to RT. DMF (2 mL) was added and the reaction mixture was stirred for 2 d. The mixture was re-cooled to 0° C. and additional tert-butyldimethylsilyl trifluoromethane-sulfonate (10.5 mL) was added, followed by the addition off 2,6-lutidine (10.6 ml). The reaction mixture was stirred at RT overnight, then washed with saturated NH₄Cl and 0.5 M aq HCl. The organic layer was concentrated in vacuo to a crude residue which was dissolved in EtOAc and then washed with several portions of dilute HCl until the pH of the aqueous wash was approximately 3. The organic layer was dried over Na₂SO₄ and concentrated in vacuo. Purification by silica gel chromatography gave the title compound in 64% yield.

LC/MS (ES⁺)[(M+H)⁺]: 718 for C₃₄H₄₂F₃N₅O₅SSi

¹H NMR (DMSO-d₆): δ15.00 (s, 1H); 9.49 (s, 1H); 8.86 (s, 1H); 8.57 (m, 1H); 8.47 (d, 1H); 8.39 (s, 1H); 8.33 (m, 1H); 8.19 (s, 1H); 7.79 (dd, 1H); 7.63 (m, 1H); 5.24 (m, 1H); 4.25 (m, 2H); 3.21 (m, 2H); 1.11 (t, 3H); 1.02 (s, 9H); 0.60 (s, 9H); −0.02 (s, 3H); −0.06 (s, 3H).

### Intermediate 8

### 6-{6-[(ethylcarbamoyl)amino]-4-[4-(trifluoromethyl)-1,3-thiazol-2-yl]pyridin-3-yl}-1-[(1S)-1-(hydroxymethyl)-2,2-dimethylpropyl]-4-oxo-1,4-dihydroquinoline-3-carboxylic acid

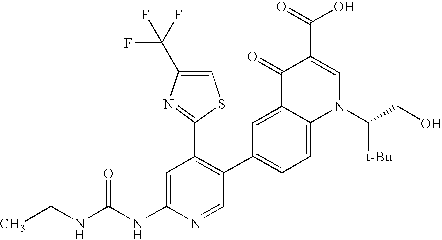

To a solution of (S)-ethyl 6-(6-(3-ethylureido)-4-(4-(trifluoromethyl)thiazol-2-yl)pyridin-3-yl)-1-(1-hydroxy-3,3-dimethylbutan-2-yl)-4-oxo-1,4-dihydroquinoline-3-carboxylate (Intermediate 9, 212.7 mg, 0.34 mmol) in tetrahydrofuran (1 mL) and methanol (1 mL) was added 2 M lithium hydroxide (0.253 mL, 0.51 mmol). The reaction mixture was heated at 100° C. for 15 min in a microwave reactor. The reaction mixture was cooled to room temperature and diluted with water. 1 N HCl was added until pH 3-4 was reached. The precipitate that formed was collected by filtration, washed with water and hexanes. Purification via column chromatography (silica, 95:5 methylene chloride/methanol) provided 6-{6-[(ethylcarbamoyl)amino]-4-[4-(trifluoromethyl)-1,3-thiazol-2-yl]pyridin-3-yl}-1-[(1S)-1-(hydroxymethyl)-2,2-dimethylpropyl]-4-oxo-1,4-dihydroquinoline-3-carboxylic acid as a light yellow solid.

MS (ESP): 604 (M+1) for C₂₈H₂₈F₃N₅O₅S.

¹H NMR (d₆-DMSO) δ 15.01 (s, 1H), 9.4 (s, 1H), 8.78 (s, 1H), 8.46 (s, 1H), 8.37 (d, 1H), 8.31 (s, 1H), 8.25 (d, 1H), 8.11 (s, 1H), 7.72 (dd, 1H), 7.5 (t, 1H), 5.12-5.08 (m, 1H), 4.09-3.96 (m, 2H), 3.17-3.08 (m, 2H), 1.02 (t, 3H), 0.89 (s, 9H).

### Intermediate 9

(S)-ethyl 6-(6-(3-ethylureido)-4-(4-(trifluoromethyl)thiazol-2-yl)pyridin-3-yl)-1-(1-hydroxy-3,3-dimethylbutan-2-yl)-4-oxo-1,4-dihydroquinoline-3-carboxylate

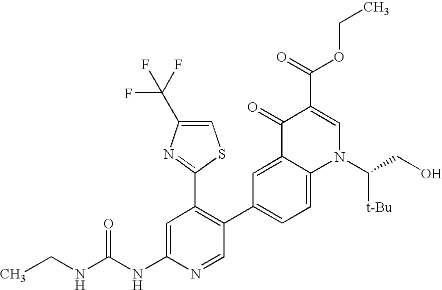

To a solution of palladium (II) acetate (28.0 mg, 0.12 mmol, 0.1 equiv.) and 1,1′-bis(di-t-butylphosphino)ferrocene (59.1 mg, 0.12 mmol, 0.1 equiv.) in acetonitrile (3 mL) was added (S)-ethyl 1-(1-hydroxy-3,3-dimethylbutan-2-yl)-6-iodo-4-oxo-1,4-dihydroquinoline-3-carboxylate (Intermediate 10, 500 mg, 1.25 mmol), followed by 6-(3-ethylureido)-4-(4-(trifluoromethyl)thiazole-2-yl)pyridine-3-ylboronic acid (Intermediate 12, 453 mg, 1.26 mmol) and a solution of potassium carbonate (258 mg, 1.87 mmol, 1.5 equiv.) in water (1 mL). The reaction mixture was stirred at 60° C. for 2 h, cooled to room temperature, and diluted with water (2 mL). The precipitate that formed was collected by filtration, washed with water and hexanes and dried to provide (S)-ethyl 6-(6-(3-ethylureido)-4-(4-(trifluoromethyl)thiazol-2-yl)pyridin-3-yl)-1-(1-hydroxypropan-2-yl)-4-oxo-1,4-dihydroquinoline-3-carboxylate as a grey solid (583 mg, 79%).

MS (ESP): 632 for C₃₀H₃₂F₃N₅O₅S

¹H NMR (d₆-DMSO) δ 9.45 (s, 1H), 8.65 (s, 1H), 8.53 (s, 1H), 8.34 (s, 1H), 8.21-8.17 (m, 3H), 7.63 (d, 2H), 5.12-5.09 (m, 1H), 4.97-4.92 (m, 1H), 4.29-4.2 (m, 2H), 4.05-4.0 (m, 2H), 3.26-3.18 (m, 2H), 1.28 (t, 3H), 1.11 (t, 3H), 0.97 (s, 9H).

### Intermediate 10

(S)-ethyl 1-(1-hydroxy-3,3-dimethylbutan-2-yl)-6-iodo-4-oxo-1,4-dihydroquinoline-3-carboxylate

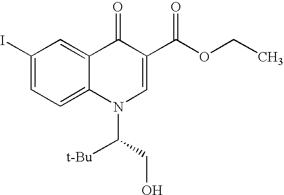

To a solution of ethyl 3-(dimethylamino)-2-(2-fluoro-5-iodobenzoyl)acrylate (Intermediate 11, 1 g, 2.56 mmol) dissolved in THF (10 mL) was added ((S)-2-amino-3,3-dimethylbutan-1-ol (300 mg, 2.56 mmol). The reaction mixture was stirred at 60° C. for 2 h. The reaction mixture was concentrated under reduced pressure, and the resulting residue was suspended in DMF (10 mL). Potassium carbonate was added (1.06 g, 7.67 mmol, 3 equiv.), and the reaction mixture was stirred at 70° C. for 18 h. The reaction mixture was cooled down to room temperature and quenched with 1 N HCl. The precipitate that formed was collected by filtration, washed with water and hexanes to provide (R)-ethyl 1-(1-hydroxypropan-2-yl)-6-iodo-4-oxo-1,4-dihydroquinoline-3-carboxylate (446 mg, 39%).

MS (ESP): 443 for C₁₈H₂₂INO₄

¹H NMR (d₆-DMSO) δ 8.62 (s, 1H), 8.53 (s, 1H), 8.01 (s, 1H), 5.07-4.99 (m, 1H), 4.92-4.85 (m, 1H), 4.26-4.20 (m, 2H), 4.02-3.95 (m, 2H), 1.28 (t, 3H), 0.95 (s, 9H).

### Intermediate 11

### Z)-Ethyl 3-(dimethylamino)-2-(2-fluoro-5-iodobenzoyl)acrylate

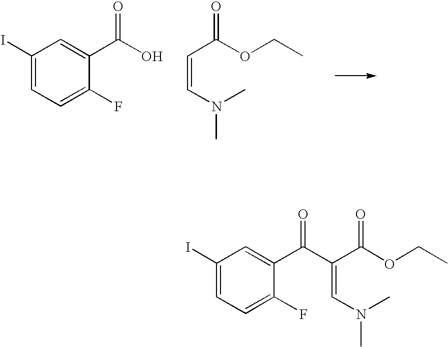

2-Fluoro-5-iodobenzoic acid (5.20 g, 19.55 mmol, Aldrich) was suspended in thionyl chloride (1.427 mL, 19.55 mmol, Aldrich) and heated to reflux for 30 min. The reaction mixture was cooled to room temperature and concentrated under reduced pressure to a tan solid. The solid was dissolved in toluene (30 mL) and concentrated under reduced pressure again. To a solution of the resulting solid (5.20 g, 19.55 mmol) in toluene (30 mL) was added triethylamine (2.72 mL, 19.55 mmol, Acros) and (Z)-ethyl 3-(dimethylamino)acrylate (3.64 mL, 25.41 mmol, Acros) and the reaction mixture was heated to 90° C. for 1 h. The reaction mixture was diluted with water and extracted three times with ethyl acetate. The combined organic extracts were dried over magnesium sulfate, filtered and concentrated under reduced pressure. Purification by Isco column (0%-100% ethyl acetate/dichloromethane) afforded the desired compound as a yellow solid (6.51 g).

MS (ES) (M+H)⁺: 392 for C₁₄H₁₅FINO₃

¹H NMR: 0.88 (t, 3H), 2.78 (s, 3H), 3.33 (s, 3H), 3.85 (q, 2H), 7.03 (dd, 1H), 7.65 (dd, 1H), 7.77 (s, 2H).

### Intermediate 12

### 1-Ethyl-3-(5-(4,4,5,5-tetramethyl-1,3,2-dioxaborolan-2-yl)-4-(4-(trifluoromethyl)thiazol-2-yl)pyridin-2-yl)urea

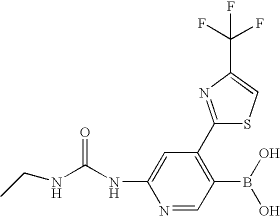

1-(5-Bromo-4-(4-(trifluoromethyl)thiazol-2-yl)pyridin-2-yl)-3-ethylurea (Intermediate 13, 200 mg, 0.51 mmol), 4,4,4′,4′,5,5,5′,5′-octamethyl-2,2′-bi(1,3,2-dioxaborolane) (386 mg, 1.52 mmol), potassium acetate (149 mg, 1.52 mmol), and 1,1′-bis(diphenylphosphino)ferrocene-palladium dichloride (20.72 mg, 0.03 mmol) were taken in a microwave vial and degassed with argon. DMSO (4 mL) was added to the vial and the solution was heated at 90° C. for 5 h. The reaction mixture was partitioned between water and ethyl acetate. The layers were separated and the organic layer was back extracted three times with ethyl acetate. The organic layers were combined and washed with water and brine, then dried over magnesium sulfate and concentrated under reduced pressure to give a light brown solid that was a mixture of 1-Ethyl-3-(5-(4,4,5,5-tetramethyl-1,3,2-dioxaborolan-2-yl)-4-(4-(trifluoromethyl)thiazol-2-yl)pyridin-2-yl)urea (Intermediate 12A, 35%), {6-{[(ethylamino)carbonyl]amino}-4-[4-(trifluoromethyl)-1,3-thiazol-2-yl]pyridin-3-yl}boronic acid (25%) and N-ethyl-N′-{4-[4-(trifluoromethyl)-1,3-thiazol-2-yl]pyridin-2-yl}urea (25%). The crude mixture was taken to the next step without further purification.

LC/MS (ES⁺)_{[}(M+H)⁺]: 361 for C₁₂HᵤBF₃N₄O₃S. ¹H NMR (300 MHz, d₆-DMSO): 1.09 (t, 3H), 3.12 (m, 2H), 7.82 (t, 1H), 7.95 (s, 1H), 8.17 (s, 2H), 8.31 (s, 1H), 8.65 (s, 1H), 9.35 (s, 1H).

In a subsequent step, the crude solid mixture was suspended in 2 L of water and 500 mL of 6N HCl, and heated to 100° C. Upon hydrolysis, the boronic acid dissolves in water at that temperature. Insoluble orange-yellow colored material was observed. This was removed by filtering the hot reaction mixture (Buchner funnel). The hydrolysis was monitored by LC/MS, cooled to room temperature, and adjusted to pH=5.5 with 20% NaOH. The pure boronic acid precipitated from the solution. The product (Intermediate 12) was filtered, washed with water, and then dried in vacuo at 100° C.

MS (ESP): 361 (MH⁺) for C₁₂H₁₂BF₃N₄O₃S

¹H NMR (d₆-DMSO): δ 1.11 (t, 3H), 3.16-3.22 (q, 2H), 7.71 (bt, 1H), 7.89 (d, 1H), 8.16 (s, 1H), 8.28 (s, 1H), 8.63 (s, 1H), 9.26 (s, 1H).

### Intermediate 13

### 1-(5-bromo-4-(4-trifluoromethylthiazol-2-yl)pyridin-2-yl)-3-ethylurea

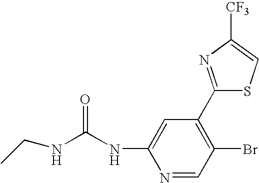

5-Bromo-2-(3-ethylureido)pyridine-4-carbothioamide (Intermediate 14, 6.06 g, 20 mmol) was suspended in acetonitrile (150 ml). 3-Bromo-1,1,1-trifluoroacetone (30 mmol) was added and the reaction mixture was heated at reflux for 16 h. The reaction was cooled to 0° C. and the suspension was filtered. The solid was washed with acetonitrile (50 ml), collected, and dried in a vacuum oven at 50° C. for 4 hours to give an off white solid.

MS (ESP): 395 (M+H⁺) for C₁₂H₁₀BrF₃N₄OS

¹H NMR: 1.1 (t, 3H), 3.20 (q, 2H), 7.23 (s, 1H), 8.40 (s, 1H), 8.60 (s, 1H), 8.83 (s, 1H), 9.40 (s, 1H).

### Intermediate 14

### 5-Bromo-2-(3-ethylureido)pyridine-4-carbothioamide

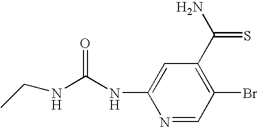

A solution of 5-bromo-2-(3-ethylureido)isonicotinamide (Intermediate 15, 52.17 g, 181.73 mmol), Lawesson's Reagent (73.50 g, 181.73 mmol), and tetrahydrofuran (840 mL) was prepared and stirred with heating at reflux overnight. After approximately 12 h, it was confirmed by LCMS that the reaction had gone to completion; therefore, stirring was stopped and a bright yellow precipitate was allowed to settle to ease filtration. The precipitate was then filtered and subsequently washed with 500 mL of additional tetrahydrofuran. The solid was then dried in the vacuum oven at 50° C. for 30 min, yielding 51 g of a bright yellow solid (92.5%).

MS (ESP): 304 (M+H⁺) for C₉H₁₁BrN₄OS

¹H NMR (DMSO-d₆): δ 1.1 (t, 3H), 3.18 (q, 2H), 7.38 (s, 1H), 7.50 (s, 1H), 8.28 (s, 1H), 9.25 (s, 1H), 9.80 (s, 1H), 10.28 (s, 1H).

### Intermediate 15

### 5-Bromo-2-(3-ethylureido)isonicotinamide

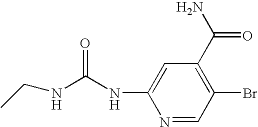

A solution of methyl 5-bromo-2-(3-ethylureido)isonicotinate (Intermediate 16, 56.22 g, 186.0 mmol) in 7N ammonia in methanol (1 L) was allowed to stir at room temperature in a sealed flask for 1½ d. The precipitate that formed was collected by filtration, rinsed with acetonitrile (500 mL), and then dried on the high vacuum pump overnight, yielding 50.8 g of a solid (95.1%).

MS (ESP): 287 (M+H⁺) for C₉H₁₁BrN₄O₂

¹H NMR (DMSO-d₆): δ 1.1 (t, 3H), 3.18 (q, 2H), 7.40 (s, 1H), 7.60 (s, 1H), 7.80 (s, 1H), 8.1 (s, 1H), 8.38 (s, 1H), 9.39 (s, 1H).

### Intermediate 16

### Methyl 5-bromo-2-(3-ethylureido)isonicotinate

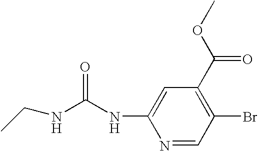

A solution of methyl 2-amino-5-bromoisonicotinate (50 g, 216.5 mmol) in chloroform (500 mL) was placed into a sealed tube. Ethyl isocyanate (51 mL, 649.4 mmol) was then added in two parts over the course of 6 hours. The sealed tube was insulated and heated at 40° C. for 3 d. The reaction mixture was then cooled to room temperature, concentrated under reduced pressure, and extracted with ethyl acetate (3 L) and water (1 L). The extracted organic layer was then dried with sodium sulfate, filtered, and concentrated under reduced pressure to yield 68.4 g of a pale yellow solid (96%).

MS (ESP): 302 (M+H⁺) for C₁₀H₁₂BrN₃O₃

¹H NMR (CDCl₃): δ 1.22 (t, 3H), 3.41 (q, 2H), 7.22 (s, 1H), 7.30 (s, 1H), 8.38 (s, 1H), 8.70 (s, 1H), 9.42 (s, 1H).

### Intermediate 17

1-((2S)-1-(tert-butoxy(hydroxy)phosphoryloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylic acid

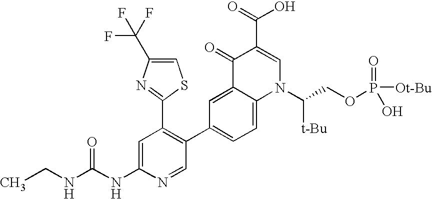

To a solution of (S)-ethyl 1-(1-(di-tert-butoxyphosphoryloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylate (Intermediate 22, 1.32 g, 1.6 mmol, 1 equiv.) in methanol (2.5 mL) and THF (2.5 mL) was added 2 M lithium hydroxide (1.6 mL, 3.2 mmol, 2 equiv.). This was stirred in a microwave at 100° C. for 15 min. The reaction mixture was diluted with water, and 1 N HCl (1 mL) was added. The reaction mixture was partitioned between water (10 mL) and ethyl acetate (10 mL). The aqueous layer was extracted with ethyl acetate (2×10 mL), and the organic layers were dried over sodium sulfate then concentrated to provide 1-((2S)-1-(tert-butoxy(hydroxy)phosphoryloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylic acid as a yellow solid (645 mg, 54%).

Calcd for C₃₂H₃₇F₃N₅O₈PS [M+H]⁺: 740.02.

¹H NMR (d₆-DMSO) δ 14.94 (s, 1H), 9.48 (s, 1H), 8.82 (s, 1H), 8.52 (s, 1H), 8.37 (s, 1H), 8.31 (d, 1H), 8.18 (s, 1H), 7.81 (dd, 1H), 7.6 (t, 1H), 5.44-5.4 (m, 1H), 4.54-4.38 (m, 2H), 3.24-3.15 (m, 2H), 1.16 (s, 9H), 1.09 (t, 3H), 0.98 (s, 9H).

### Intermediates 18-21

The following Intermediates were prepared according to the procedure described for Intermediate 17 from the indicated starting material.

| Int | Compound | Data | SM |
|---|---|---|---|
| 18 | 1-((2S)-1-(tert- | Calcd for C₃₁H₃₅F₃N₅O₈PS | Intermediate |
| | butoxy(hydroxy)phosphoryloxy)-3- | [M + H]⁺: 726.13 | 23 |
| | methylbutan-2-yl)-6-(6-(3-ethylureido)-4- | ¹H NMR (d₆-DMSO) δ | |
| | (4-(trifluoromethyl)thiazol-2-yl)pyridin-3- | 14.97 (s, 1H), 9.46 (s, 1H), | |
| | yl)-4-oxo-1,4-dihydroquinoline-3- | 8.97 (s, 1H), 8.5 (s, 1H), | |
| | carboxylic acid | 8.39 (s, 1H), 8.38 (d, 1H), | |
| | 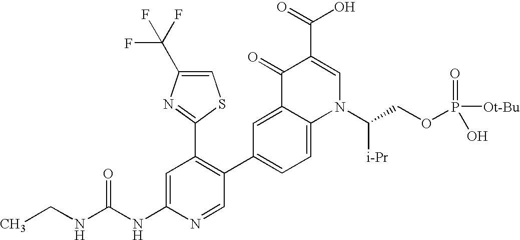 | 8.33 (d, 1H), 8.21 (s, 1H), 7.87 (dd, 1H), 7.59 (s, 1H), 5.22-5.15 (m, 1H), 4.54- 4.45 (m, 1H), 4.23-4.18 (m, 1H), 3.26-3.17 (m, 2H), 1.23 (s, 9H), 1.15 (d, 3H), 1.11 (t, 3H), 0.75 (d, 3H) | |
| 19 | 1-((2S)-1-(tert- | Calcd for C₃₀H₃₃F₃N₅O₈PS | Intermediate |
| | butoxy(hydroxy)phosphoryloxy)butan-2- | [M + H]⁺: 712.02 | 24 |
| | yl)-6-(6-(3-ethylureido)-4-(4- | ¹H NMR (d₆-DMSO) δ | |
| | (trifluoromethyl)thiazol-2-yl)pyridin-3-yl)- | 15.01 (s, 1H), 9.48 (s, 1H), | |
| | 4-oxo-1,4-dihydroquinoline-3-carboxylic | 8.92 (s, 1H), 8.5 (d, 1H), | |
| | acid | 8.37 (s, 1H), 8.34 (d, 1H), | |
| | 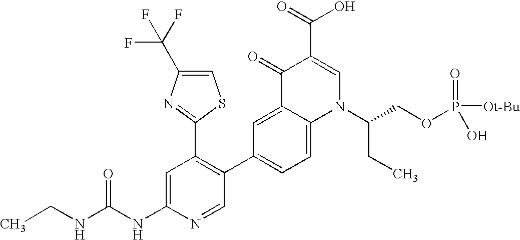 | 8.22 (s, 1H), 7.9 (dd, 1H), 7.61 (t, 1H), 5.4-5.35 (m, 1H), 4.41-4.32 (m, 1H), 4.2-4.14 (m, 1H), 3.25-3.17 (m, 2H), 2.11-2.01 (m, 2H), 1.21 (s, 9H), 1.11 (t, 3H), 0.89 (t, 3H) | |
| 20 | 1-((2R)-1-(tert- | Calcd for C₃₀H₃₃F₃N₅O₈PS | Intermediate |
| | butoxy(hydroxy)phosphoryloxy)butan-2- | [M + H]⁺: 712.02 | 25 |
| | yl)-6-(6-(3-ethylureido)-4-(4- | ¹H NMR (d₆-DMSO) δ | |
| | (trifluoromethyl)thiazol-2-yl)pyridin-3-yl)- | 15.03 (s, 1H), 9.49 (s, 1H), | |
| | 4-oxo-1,4-dihydroquinoline-3-carboxylic | 8.91 (2, 1H), 8.5 (s, 1H), | |
| | acid | 8.37 (s, 1H), 8.32 (d, 1H), | |
| | 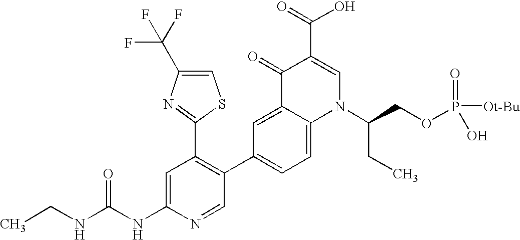 | 8.3 (d, 1H), 8.22 (s, 1H), 7.85 (dd, 1H), 7.63 (t, 1H), 5.38-5.34 (m, 1H), 4.27- 4.09 (m, 2H), 3.25-3.17 (m, 2H), 2.09-1.92 (m, 2H), 1.17 (s, 9H), 1.11 (t, 3H), 0.88 (t, 3H) | |
| 21 | 1-((2S)-1-(tert- | Calcd for C₃₂H₃₇F₃N₅O₈PS | Intermediate |
| | butoxy(hydroxy)phosphoryloxy)-4- | [M + H]⁺: 739.94 | 26 |
| | methylpentan-2-yl)-6-(6-(3-ethylureido)-4- | ¹H NMR (d₆-DMSO) δ | |
| | (4-(trifluoromethyl)thiazol-2-yl)pyridin-3- | 14.95 (s, 1H), 9.46 (s, 1H), | |
| | yl)-4-oxo-1,4-dihydroquinoline-3- | 8.91 (s, 1H), 8.5 (s, 1H), | |
| | carboxylic acid | 8.38 (s, 1H), 8.36 (d, 1H), | |
| | 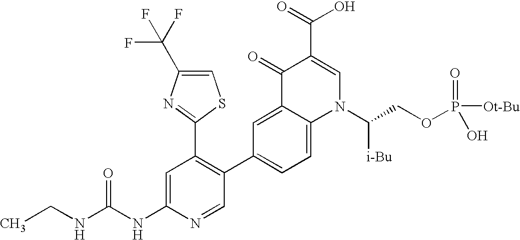 | 8.33 (d, 1H), 8.22 (s, 1H), 7.9 (dd, 1H), 7.58 (t, 1H), 5.47-5.43 (m, 1H), 4.35- 4.27 (m, 1H), 4.19-4.14 (m, 1H), 3.25-3.17 (m, 2H), 2.09-2.0 (m, 1H), 1.93-1.89 (m, 1H), 1.48-1.43 (m, 1H), 1.22 (s, 9H), 1.11 (t, 3H), 0.92 (d, 3H), 0.87 (d, 3H) | |

### Intermediate 22

(S)-ethyl 1-(1-(di-tert-butoxyphosphoryloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylate

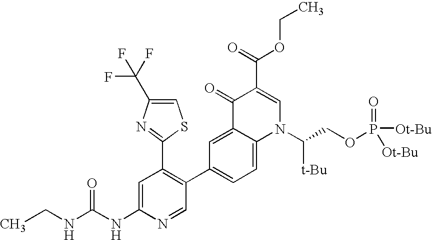

To a solution of (S)-ethyl 6-(6-(3-ethylureido)-4-(4-(trifluoromethyl)thiazol-2-yl)pyridin-3-yl)-1-(1-hydroxy-3,3-dimethylbutan-2-yl)-4-oxo-1,4-dihydroquinoline-3-carboxylate (Intermediate 9, 1.475 g, 2.34 mmol, 1 equiv.) in DMF (12 mL) was added 1H-tetrazole (10.38 mL, 4.67 mmol, 2 equiv.) followed by di-tert-butyl diethylphosphoramidite (1.37 mL, 4.9 mmol, 2.1 equiv.). The reaction was stirred at room temperature for 4 hr. The reaction was cooled down to −10° C. and 30% hydrogen peroxide (0.52 mL, 4.9 mmol, 2.15 equiv.) was added. The reaction was stirred at room temperature for 3 hr, then cooled to 0° C. and a solution of sodium metabisulfite (2.7 g, 14.2 mmol, 6 equiv.) in water (4 mL) was added. The reaction was stirred at room temperature for 30 min, then partitioned between water (20 mL) and ethyl acetate (20 mL). The aqueous layer was extracted with ethyl acetate (2×20 mL), and the organic layers were washed with brine (20 mL), dried over sodium sulfate, and concentrated. The compounds was purified via silica gel chromatography and concentrated to provide (S)-ethyl 1-(1-(di-tert-butoxyphosphoryloxy)-3,3-dimethylbutan-2-yl)-6-(6-(3-ethylureido)-4-(4-(trifluoromethyl)thiazol-2-yl)pyridin-3-yl)-4-oxo-1,4-dihydroquinoline-3-carboxylate as a orange solid (1.32 g, 69%).

Calcd for C₃₈H₄₉F₃N₅O₈PS [M+H]⁺: 824.05.

¹H NMR (d₆-DMSO) δ 9.46 (s, 1H), 8.63 (s, 1H), 8.55 (s, 1H), 8.33 (s, 1H), 8.27 (d, 1H), 8.19 (s, 1H), 8.18 (d, 1H), 7.68 (dd, 1H), 7.61 (t, 1H), 5.26-5.22 (m, 1H), 4.56-4.41 (m, 2H), 4.27-4.2 (m, 2H), 3.26-3.17 (m, 2H), 1.27 (t, 3H), 1.26 (s, 18H), 1.11 (t, 3H), 1.02 (s, 9H).

### Intermediate 23-26

The following Intermediates were prepared according to the procedure described for Intermediate 22 from the indicated starting material.

| Int | Compound | Data | SM |
|---|---|---|---|
| 23 | (S)-ethyl 1-(1-(di-tert- | Calcd for C₃₇H₄₇F₃N₅O₈PS | Intermediate |
| | butoxyphosphoryloxy)-3-methylbutan-2- | [M + H]⁺: 810.0 | 27 |
| | yl)-6-(6-(3-ethylureido)-4-(4- | ¹H NMR (d₆-DMSO) δ 9.44 | |
| | (trifluoromethyl)thiazol-2-yl)pyridin-3- | (s, 1H), 9.36 (s, 1H), 8.65 (s, | |
| | yl)-4-oxo-1,4-dihydroquinoline-3- | 1H), 8.52 (s, 1H), 8.33 (s, | |
| | carboxylate | 1H), 8.2 (s, 1H), 8.17 (d, 1H), | |
| | 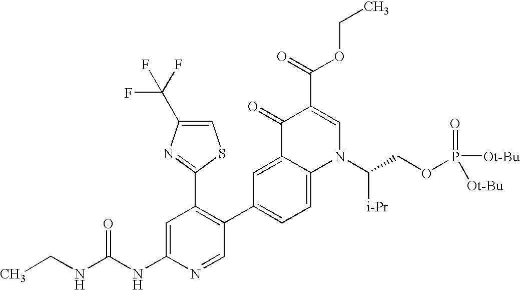 | 7.7 (dd, 1H), 7.61 (t, 1H), 5.0-4.5 (m, 1H), 4.55-4.4 (m, 1H), 4.27-4.2 (m, 2H), 4.22- 4.15 (m, 1H), 3.26-3.19 (m, 2H), 2.43-2.32 (m, 1H), 1.28 (s, 18H), 1.27 (t, 3H), 1.16 (d, 3H), 1.11 (t, 3H), 0.78 (d, 3H) | |
| 24 | (S)-ethyl 1-(1-(di-tert- | Calcd for C₃₆H₄₅F₃N₅O₈PS | Intermediate |
| | butoxyphosphoryloxy)butan-2-yl)-6-(6- | [M + H]⁺: 796.13 | 28 |
| | (3-ethylureido)-4-(4- | ¹H NMR (d₆-DMSO) δ 9.45 | |
| | (trifluoromethyl)thiazol-2-yl)pyridin-3- | (s, 1H), 8.62 (s, 1H), 8.51 (s, | |
| | yl)-4-oxo-1,4-dihydroquinoline-3- | 1H), 8.31 (s, 1H), 8.21 (s, | |
| | carboxylate | 1H), 8.17 (d, 1H), 8.12 (d, | |
| | 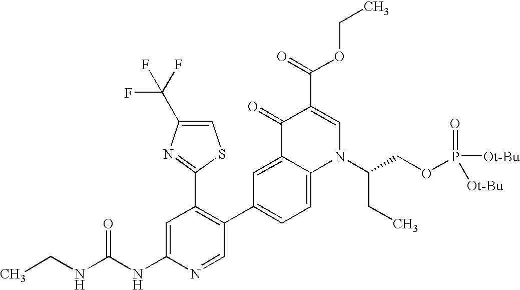 | 1H), 7.73 (dd, 1H), 7.63 (t, 1H), 5.23-5.19 (m, 1H), 4.34- 4.29 (m, 1H), 4.27-4.2 (m, 2H), 4.2-4.14 (m, 1H), 3.23- 3.19 (m, 2H), 2.08-1.91 (2H), 1.28 (t, 3H), 1.26 (s, 18H), 1.11 (t, 3H), 0.89 (t, 3H) | |
| 25 | (R)-ethyl 1-(1-(di-tert- | Calcd for C₃₆H₄₅F₃N₅O₈PS | Intermediate |
| | butoxyphosphoryloxy)butan-2-yl)-6-(6- | [M + H]⁺: 796.21 | 29 |
| | (3-ethylureido)-4-(4- | | |
| | (trifluoromethyl)thiazol-2-yl)pyridin-3- | | |
| | yl)-4-oxo-1,4-dihydroquinoline-3- | | |
| | carboxylate | | |
| | 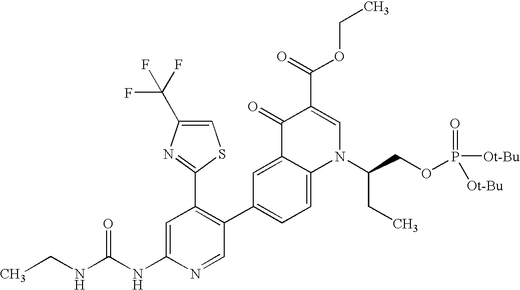 | | |
| 26 | (S)-ethyl 1-(1-(di-tert- | Calcd for C₃₈H₄₉F₃N₅O₈PS | Intermediate |
| | butoxyphosphoryloxy)-4-methylpentan-2- | [M + H]⁺: 824.11 | 30 |
| | yl)-6-(6-(3-ethylureido)-4-(4- | ¹H NMR (d₆-DMSO) δ 9.45 | |
| | (trifluoromethyl)thiazol-2-yl)pyridin-3- | (s, 1H), 8.61 (s, 1H), 8.61 (s, | |
| | yl)-4-oxo-1,4-dihydroquinoline-3- | 1H), 8.52 (s, 1H), 8.32 (s, | |
| | carboxylate | 1H), 8.2 (s, 1H), 8.17 (d, 1H), | |
| | 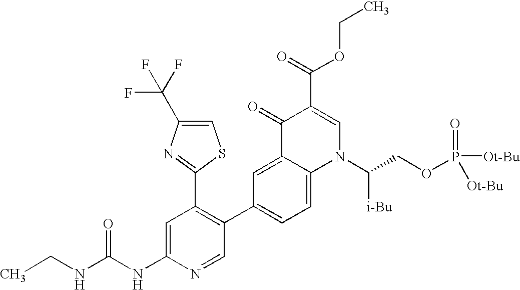 | 8.14 (d, 1H), 7.73 (dd, 1H), 7.62 (t, 1H), 5.3-5.24 (m, 1H), 4.27-4.2 (m, 2H), 4.19- 4.12 (m, 2H), 3.25-3.16 (m, 2H), 2.04-1.94 (m, 1H), 1.85- 1.78 (m, 1H), 1.49-1.43 (m, 1H), 1.28 (t, 3H), 1.26 (s, 18H), 1.11 (t, 3H), 0.92 (d, 3H), 0.87 (d, 3H) | |

### Intermediates 27-30

The following Intermediates were prepared according to the procedure described for Intermediate 9 from the starting materials indicated.

| Int | Compound | Data | SM |
|---|---|---|---|
| 27 | (S)-ethyl 6-(6-(3-ethylureido)-4-(4- | MS (ESP): 618 for | Intermediate |
| | (trifluoromethyl)thiazol-2-yl)pyridin- | C₂₉H₃₀F₃N₅O₅S | 31 and |
| | 3-yl)-1-(1-hydroxy-3-methylbutan-2- | NMR (d₆-DMSO) δ 9.44 (s, | Intermediate |
| | yl)-4-oxo-1,4-dihydroquinoline-3- | 1H), 8.72 (s, 1H), 8.51 (s, 1H), | 12A |
| | carboxylate | 8.33 (s, 1H), 8.2 (s, 1H), 8.17 | |
| | 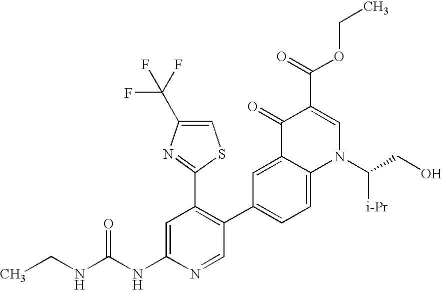 | (d, 1H), 8.11 (d, 1H), 7.62 (t, 2H), 5.22-5.17 (m, 1H), 4.68 (s, 1H), 4.27-4.2 (m, 2H), 3.95- 3.91 (m, 1H), 3.8-3.74 (m, 1H), 3.24-3.15 (m 4H), 2.4-2.3 (m, 1H), 1.27 (t, 3H), 1.1 (t, 3H), 1.08 (d, 3H), 0.75 (d, 3H) | |
| 28 | (S)-ethyl 6-(6-(3-ethylureido)-4-(4- | MS (ESP): 604 for | Intermediate |
| | (trifluoromethyl)thiazol-2-yl)pyridin- | C₂₈H₂₈F₃N₅O₅S | 32 and |
| | 3-yl)-1-(1-hydroxybutan-2-yl)-4-oxo- | NMR (d₆-DMSO) δ 9.45 (s, | Intermediate |
| | 1,4-dihydroquinoline-3-carboxylate | 1H), 8.66 (s, 1H), 8.51 (s, 1H), | 12A |
| | 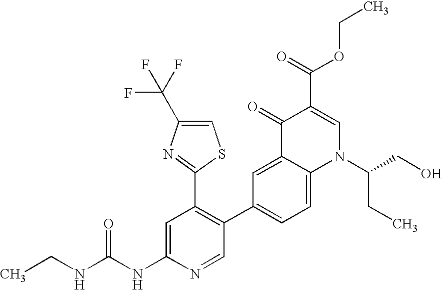 | 8.33 (s, 1H), 8.22 (s, 1H), 8.18 (d, 1H), 8.08 (d, 1H), 7.69 (dd, 1H), 7.66 (t, 1H), 5.23-5.18 (m, 1H), 4.91 (s, 1H), 4.27-4.2 (m, 2H), 3.83-3.75 (m, 2H), 3.25- 3.17 (m, 2H), 2.07-1.9 (m, 2H), 1.28 (t, 3H), 1.11 (t, 3H), 0.88 (t, 3H) | |
| 29 | (R)-ethyl 6-(6-(3-ethylureido)-4-(4- | MS (ESP): 604 for | Intermediate |
| | (trifluoromethyl)thiazol-2-yl)pyridin- | C₂₈H₂₈F₃N₅O₅S | 33 and |
| | 3-yl)-1-(1-hydroxybutan-2-yl)-4-oxo- | NMR (d₆-DMSO) δ 9.45 (s, | Intermediate |
| | 1,4-dihydroquinoline-3-carboxylate | 1H), 8.66 (s, 1H), 8.51 (s, 1H), | 12A |
| | 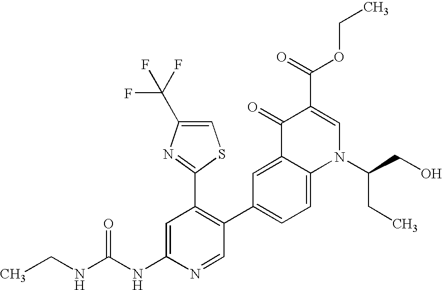 | 8.33 (s, 1H), 8.22 (s, 1H), 8.19 (d, 1H), 8.06 (d, 1H), 7.7-7.65 (m, 2H), 5.23-5.18 (m, 1H), 4.91 (s, 1H), 4.28-4.21 (m, 2H), 3.85-3.73 (m, 2H), 3.25-3.17 (m, 2H), 2.0-1.9 (m, 2H), 1.28 (t, 3H), 1.11 (t, 3H), 0.89 (t, 3H) | |
| 30 | (S)-ethyl 6-(6-(3-ethylureido)-4-(4- | MS (ESP): 632 for | Intermediate |
| | (trifluoromethyl)thiazol-2-yl)pyridin- | C₃₀H₃₂F₃N₅O₅S | 34 and |
| | 3-yl)-1-(1-hydroxy-4-methylpentan-2- | NMR (d₆-DMSO) δ 9.45 (s, | Intermediate |
| | yl)-4-oxo-1,4-dihydroquinoline-3- | 1H), 8.64 (s, 1H), 8.52 (s, 1H), | 12A |
| | carboxylate | 8.33 (s, 1H), 8.21 (s, 1H), 8.18 | |
| | 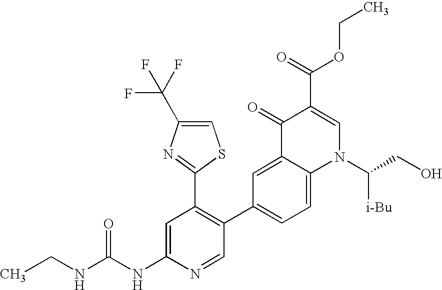 | (d, 1H), 8.1 (d, 1H), 7.69-7.61 (m, 2H), 5.21-5.18 (m, 1H), 5.02 (s, 1H), 4.27-4.21 (m, 2H), 3.78-3.71 (m, 2H), 3.25-3.17 (m, 2H), 1.92-1.84 (m, 1H), 1.78-1.74 (m, 1H), 1.49-1.42 (m, 1H), 1.28 (t, 3H), 1.11 (t, 3H), 0.92 (d, 3H), 0.86 (d, 3H) | |

### Intermediates 31-34

The following Intermediates were prepared according to the procedure described for Intermediate 10 from the starting materials indicated.

| Int | Compound | Data | SM |
|---|---|---|---|
| 31 | (S)-ethyl 1-(1-hydroxy-3-methylbutan- | MS (ESP): 430 for C₁₇H₂₀INO₄ | Intermediate |
| | 2-yl)-6-iodo-4-oxo-1,4- | ¹H NMR (d₆-DMSO) δ 8.69 (s, | 11 & (S)-2- |
| | dihydroquinoline-3-carboxylate | 1H), 8.53 (d, 1H), 8.04 (dd, | amino-3- |
| | 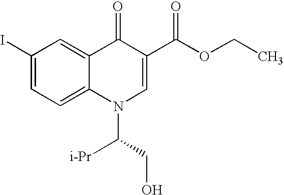 | 1H), 7.9 (d, 1H), 5.25-5.15 (m, 1H), 4.65-4.58 (m, 1H), 4.26- 4.19 (m, 2H), 3.92-3.86 (m, 1H), 3.77-3.72 (m, 1H), 2.4-2.3 (m, 1H), 1.27 (t, 3H), 1.1 (d, 3H), 0.71 (d, 3H) | methylbutan- 1-ol |
| 32 | (S)-ethyl 1-(1-hydroxy-3-methylbutan- | MS (ESP): 416 for | Intermediate |
| | 2-yl)-6-iodo-4-oxo-1,4- | C₁₆H₁₈INO₄ | 11 & (S)-2- |
| | dihydroquinoline-3-carboxylate | ¹H NMR (d₆-DMSO) δ 8.62 (s, | aminobutan- |
| | 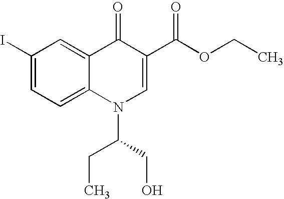 | 1H), 8.53 (s, 1H), 8.06 (d, 1H), 7.85 (d, 1H), 4.83 (s, 1H), 4.25- 4.2 (m, 2H), 3.78-3.71 (m, 2H), 1.99-1.84 (m, 2H), 1.27 (t, 3H), 0.84 (t, 3H) | 1-ol |
| 33 | (R)-ethyl 1-(1-hydroxy-3- | MS (ESP): 416 for | Intermediate |
| | methylbutan-2-yl)-6-iodo-4-oxo-1,4- | C₁₆H₁₈INO₄ | 11 & (R)-2- |
| | dihydroquinoline-3-carboxylate | NMR (d₆-DMSO) δ 8.62 (s, | aminobutan- |
| | 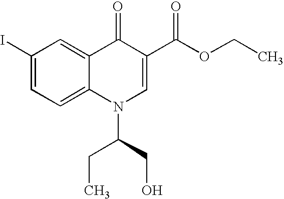 | 1H), 8.53 (s, 1H), 8.03 (dd, 1H), 7.85 (d, 1H), 4.83 (s, 1H), 4.26-4.19 (m, 2H), 3.77-3.73 (m, 2H), 1.99-1.83 (m, 2H), 1.28 (t, 3H), 0.84 (t, 3H) | 1-ol |
| 34 | (S)-ethyl 1-(1-hydroxy-4- | MS (ESP): 444 for C₁₈H₂₂INO₅ | Intermediate |
| | methylpentan-2-yl)-6-iodo-4-oxo-1,4- | H¹NMR (d₆-DMSO) δ 8.69 (s, | 11 & (S)-2- |
| | dihydroquinoline-3-carboxylate | 1H), 8.53 (d, 1H), 8.07 (dd, | amino-4- |
| | 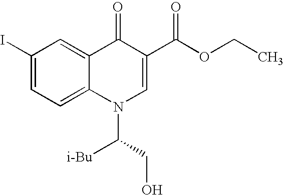 | 1H), 7.89 (d, 1H), 4.95-4.91 (m, 1H), 4.26-4.19 (m, 2H), 3.71 (d, 2H), 1.86-1.8 (m, 1H), 1.75-1.72 (m, 1H), 1.46-1.39 (m, 1H), 1.27 (t, 3H), 0.89 (d, 3H), 0.84 (d, 3H) | methylpentan- 1-ol |

### Intermediate 35

### Benzyl 2-(di-tert-butoxyphosphoryloxy)ethylcarbamate

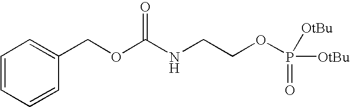

A solution of di-tert-butyl diisopropylphosphoramidite (12.59 g, 45.4 mmol) in dry tetrahydrofuran (50 mL) was treated with 1H-tetrazole (4.13 g, 59 mmol) and allowed to stir for 40 min. at room temperature. A solution of benzyl 2-hydroxyethylcarbamate (5 g, 25.6 mmol) in tetrahydrofuran (12 mL) was added dropwise rapidly and the mixture stirred for 2 h. The solution was cooled to 10° C. and treated with a solution of 70% aqueous t-butyl peroxide (12 mL) for 30 min to give a clear solution. The reaction mixture was diluted with ethyl acetate and the solution washed with 10% sodium thiosulfate solution (20 mL) and saturated sodium bicarbonate (2×15 mL) then dried over sodium sulfate, filtered and the organic layer was concentrated to a crude product. The crude product was purified by Analogix (ethyl acetate: heptane) to give the title compound.

MS (ESP): 388 (M+H⁺) for C₁₈H₃₀NO₆P

¹H NMR (300 MHz, CDCl₃): δ 1.47 (s, 18H), 3.47 (m, 2H), 4.03 (m, 2H), 4.11 (s, 2H), 5.45 (brs, 1H) 7.33 (m, 5H).

### Intermediate 36

The following Intermediate was prepared according to the procedure described for Intermediate 35 from the indicated starting material.

| Int | Compound | Data | SM |
|---|---|---|---|
| 36 | 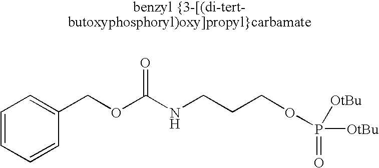 | MS (ESP): 424 (M + Na⁺) for C₁₉H₃₂NO₆P | benzyl 3- hydroxy- propyl- carbamate |

### Intermediate 37

### 2-aminoethyl di-tert-butyl phosphate

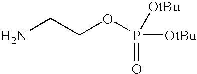

A solution of benzyl 2-(di-tert-butoxyphosphoryloxy)ethylcarbamate (Intermediate 35, 1.0 g, 2.58 mmol) was dissolved in methanol (20 mL), 10% palladium on carbon (˜5 mol %) was added and the reaction was shaken under an H₂ atmosphere at 50 PSI until the reaction was complete. The mixture was filtered through Celite, and the organics evaporated. The residue was re-dissolved in dichloromethane and filtered through a PFTE filter (4 um, ID 20 mm) and the filtrate was evaporated to give 550 mg of product.

MS (ESP): 254 (M+H⁺) for C₁₀H₂₄NO₄P.

### Intermediate 38

The following Intermediate was prepared according to the procedure described for Intermediate 37 from the indicated starting material.

| Int | Compound | Data | SM |
|---|---|---|---|
| 38 | 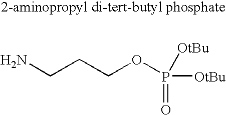 | MS (ESP): 268 (M + H⁺) for C₁₁H₂₆NO₄P ¹H NMR (300 MHz, CDCl₃): δ 1.47 (s, 18 H), 1.91 (m, 2 H), 2.88 (m, 2 H), 4.03 (m, 2 H) | Intermediate 36 |

### Intermediate 39

### ethyl 1-(1,3-dimethoxypropan-2-yl)-6-iodo-4-oxo-1,4-dihydroquinoline-3-carboxylate

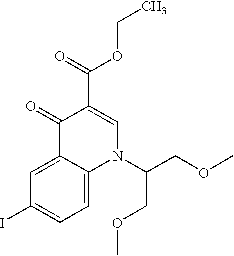

1,3-Dimethoxypropan-2-amine (1.3 g, 11.0 mmol) was added to a solution of (Z)-ethyl 3-(dimethylamino)-2-(3-iodobenzoyl)acrylate (Intermediate 11, 0.30M in THF, 30 mL, 10 mmol) and the mixture was heated at 40° C. for 3 h. The solvent was removed in vacuo and the residue was dried in a vacuum oven for 1.5 h. The residue was then dissolved in dimethyl formamide (5 mL) and potassium carbonate powder (1.5 g, 11 mmol) was added. The reaction was heated at 70° C. for a further 3 h then cooled to room temperature. The crude was placed into a refrigerator for 3 h. The solid which precipitated out was filtered, washed with a small amount of dimethyl formamide and water, then was dried to give the desired product.

MS (ESP): 446.1 (M+H⁺) for C₁₇H₂₀INO₅

¹H NMR (300 MHz, CDCl₃): δ 1.41 (t, 3H), 3.38 (s, 6H), 3.83 (d, 4H), 4.01 (q, 2H), 4.85 (m, 1H), 7.35 (d, 1H), 7.93 (d, 1H), 8.78 (s, 1H), 8.85 (s, 1H).

### Intermediate 40

The following Intermediate was prepared according to the procedure described for Intermediate 39 from the indicated starting materials.

| Int | Compound | Data | SM |
|---|---|---|---|
| 40 | 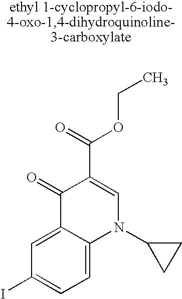 | MS (ESP): 384 (M + H⁺) for C₁₅H₁₄INO₃ ¹H NMR (300 MHz, DMSO-d₆): δ 1.10 (m, 2 H), 1.24 (m, 2 H), 1.27 (t, 3 H), 3.64 (m, 1 H), 4.22 (q, 2 H), 7.88 (d, 1 H), 8.12 (d, 1 H), 8.47 (s, 1 H), 8.50 (s, 1 H) | Intermediate 11 and cyclopropylamine |

### Intermediate 41

### ethyl 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylate

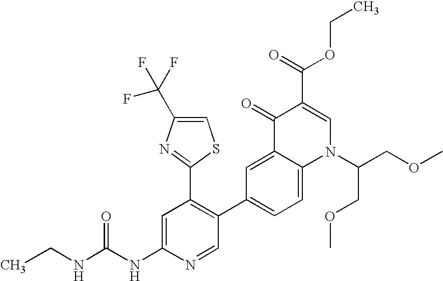

A suspension of ethyl 1-(1,3-dimethoxypropan-2-yl)-6-iodo-4-oxo-1,4-dihydroquinoline-3-carboxylate (Intermediate 39, 750 mg, 1.12 mmol) in dimethoxyethane (9 mL) in a microwave vial was stirred with heating to obtain a solution. The solution was then cooled to 30° C., trans dichlorobis(triphenylphosphine)palladium (II) (79 mg, 0.11 mmol) was added and the mixture was stirred for ˜10 min at room temperature. 6-(3-Ethylureido)-4-(4-(trifluoromethyl)thiazol-2-yl)pyridin-3-ylboronic acid (Intermediate 12, 1.3 eq) was added to the mixture followed by an aqueous solution of sodium bicarbonate (233 mg in 2.25 mL of water) and the vial was sealed. The mixture was stirred at 70° C. for ˜1 h in a heating block, then cooled to room temperature and the organic layer was passed through a PFTE filter (4 um, ID 20 mm) The volatiles were removed and the residue was re-dissolved in acetonitrile (3 mL) and placed into a refrigerator for a few hours. The solid precipitate was collected by filtration, washed with a small amount of dimethyl formamide, water and dried.

MS (ESP): 634 (M+H⁺) for C₂₉H₃₀F₃N₅O₆S.

### Intermediate 42

The following Intermediate was prepared according to the procedure described for Intermediate 41 from the starting material indicated.

| Int | Compound | Data | SM |
|---|---|---|---|
| 42 | 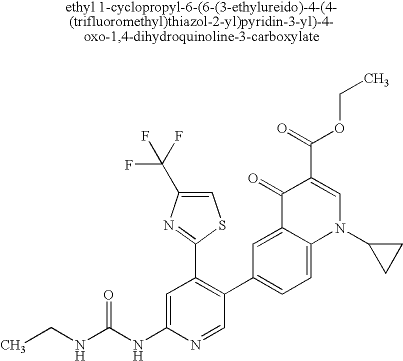 | MS (ESP): 572.2 (M + H⁺) for C₂₇H₂₄F₃N₅O₄S | Intermediate 40 and Intermediate 12A |

### Intermediate 43

### 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylic acid

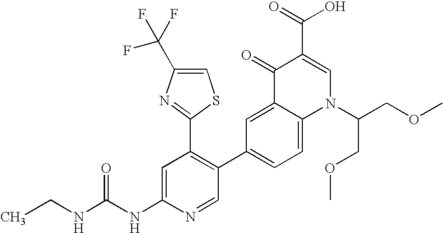

A solution of ethyl 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylate (Intermediate 41, 100 mg, 0.16 mmol) was dissolved in tetrahydrofuran and treated with 1M lithium hydroxide (240 μL) and the mixture stirred at room temperature. A precipitate started to develop and mixture was left to stir overnight. The solids were collected by filtration and washed with ethyl acetate to give 85 mg of product after drying.

MS (ESP): 606.3 (M+H⁺) for C₂₇H₂₆F₃N₅O₆S

¹H NMR (300 MHz, dmso-d₆): δ 1.12 (t, 3H), 3.22 (m, 2H), 3.26 (s, 6H), 3.85 (m, 2H), 3.94 (m, 2H), 5.58 (brs, 1H), 7.59 (t, 1H), 7.84 (d, 1H), 8.20 (s, 1H), 8.27 (d, 1H), 8.33 (s, 1H), 8.38 (s, 1H), 8.52 (s, 1H), 8.98 (s, 1H), 9.47 (s, 1H).

### Intermediate 44

The following Intermediate was prepared according to the procedure described for Intermediate 43 from the indicated starting material.

| Int | Compound | Data | SM |
|---|---|---|---|
| 44 | 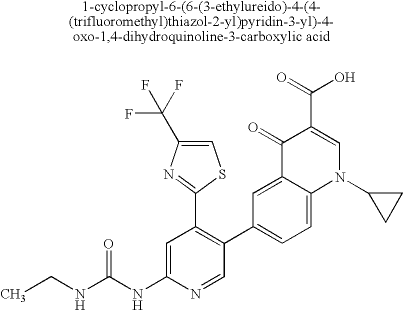 | MS (ESP): 544 (M + H⁺) for C₂₅H₂₀F₃N₅O₄S ¹H NMR (300 MHz, CD₃OD): δ 1.20 (m, 2 H), 1.22 (t, 3 H), 1.33 (m, 2 H), 3.34 (q, 2 H), 3.62 (m, 1 H), 7.63 (br, 1 H), 7.90 (s, 1 H), 8.13 (s, 1 H), 8.18 (d, 1 H), 8.31 (s, 1 H), 8.39 (s, 1 H), 8.65 (s, 1 H) | Intermediate 42 |

### Intermediate 45

### 1-(5-bromo-4-(4-cyclopropylthiazol-2-yl)pyridin-2-yl)-3-ethylurea

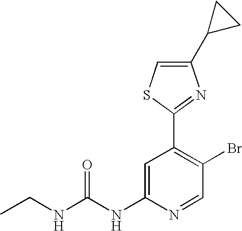

In a 25 mL flask, 5-bromo-2-(3-ethylureido)pyridine-4-carbothioamide (Intermediate 14, 6.1 g, 20.12 mmol) and 2-bromo-1-cyclopropylethanone (3.28 g, 20.12 mmol) were suspended in ethanol (40 mL). The reaction mixture was heated at 80° C. for 3 h, cooled to room temperature and concentrated to one half the volume under reduced pressure. The resulting solids were filtered and washed with acetonitrile. Isolation gave 6.1 g of the title compound as an off-white solid.

LC/MS (ES⁺)[(M+H)⁺]: 367, 369 for C₁₄H₁₅BrN₄OS.

¹H NMR (300 MHz, d₆-DMSO): 0.88 (m, 2H), 0.99 (m, 2H), 1.08 (t, 3H), 2.19 (m, 1H), 3.17 (m, 2H), 7.35 (m, 1H), 7.62 (s, 1H), 8.30 (s, 1H), 8.48 (s, 1H), 9.32 (s, 1H).

### Intermediate 46

### 4-(4-cyclopropylthiazol-2-yl)-6-(3-ethylureido)pyridin-3-ylboronic acid

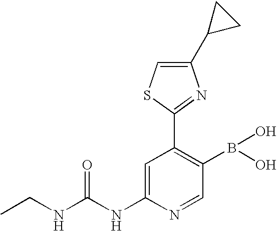

In a 100 mL glass round bottom flask, 1-(5-bromo-4-(4-cyclopropylthiazol-2-yl)pyridin-2-yl)-3-ethylurea (Intermediate 45, 2 g, 5.45 mmol) and bis(triphenylphosphine)palladium chloride (0.382 g, 0.54 mmol) were dissolved in 1,4-dioxane (30 mL). The solution was degassed and purged with nitrogen then heated to 40° C. for 10 min. 4,4,4′,4′,5,5,5′,5′-Octamethyl-2,2′-bi(1,3,2-dioxaborolane) (4.15 g, 16.34 mmol) was added in a single portion and the temperature was raised to 100° C. Triethylamine (2.27 mL, 16.34 mmol) and potassium acetate (1.603 g, 16.34 mmol) were added and the solution was heated at 100° C. for 16 h. The reaction mixture was cooled to room temperature, filtered through a pad of Celite and partitioned between water and ethyl acetate. The layers were separated and the aqueous layer was back extracted three times with ethyl acetate. The organic layers were combined and washed with water, brine, dried over sodium sulfate, concentrated under reduced pressure, and purified by silica gel flash column chromatography (9:1 CH₂Cl₂/MeOH). Isolation gave 1.05 g of the title compound as an off-white solid.

LC/MS (ES⁺)[(M+H)⁺]: 333 for C₁₄H₁₇BN₄O₃S.

### Intermediate 47

(S)-ethyl 6-(4-(4-cyclopropylthiazol-2-yl)-6-(3-ethylureido)pyridin-3-yl)-1-(1-hydroxy-4-methylpentan-2-yl)-4-oxo-1,4-dihydroquinoline-3-carboxylate

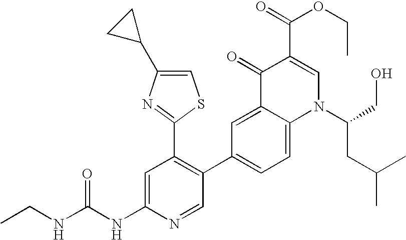

In a 50 mL pear-shaped flask, 4-(4-cyclopropylthiazol-2-yl)-6-(3-ethylureido)pyridin-3-ylboronic acid (Intermediate 46 0.167 g, 0.50 mmol), (S)-ethyl 1-(1-hydroxy-4-methylpentan-2-yl)-6-iodo-4-oxo-1,4-dihydroquinoline-3-carboxylate (Intermediate 34, 0.22 g, 0.50 mmol), 1,1′-bis(di-t-butylphosphino)ferrocene (0.024 g, 0.05 mmol), and palladium(II) acetate (0.011 g, 0.05 mmol) were combined and suspended in acetonitrile (3 mL). Potassium carbonate (0.103 g, 0.74 mmol), dissolved in 1 mL water, was then added. The suspension was degassed and purged with nitrogen, then heated at 100° C. for 60 minutes. The reaction was partitioned between water and ethyl acetate and the layers were separated. The organic phase was washed with NaHCO₃ (sat.), water, and brine, then dried over magnesium sulfate, filtered and concentrated under reduced pressure. The resulting solids were triturated with water, then collected by filtration and dried in vacuo. Isolation gave 240 mg of the title compound as an off-white solid.

LC/MS (ES⁺)[(M+H)₊]: 604 for C₃₂H₃₇N₅O₅S.

### Intermediate 48

### (S)-6-(4-(4-cyclopropylthiazol-2-yl)-6-(3-ethylureido)pyridin-3-yl)-1-(1-hydroxy-4-methylpentan-2-yl)-4-oxo-1,4-dihydroquinoline-3-carboxylic acid

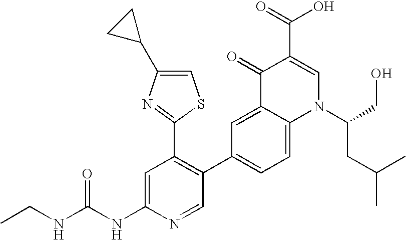

1N LiOH (1 mL) was added to a mixture of (S)-ethyl 6-(4-(4-cyclopropylthiazol-2-yl)-6-(3-ethylureido)pyridin-3-yl)-1-(1-hydroxy-4-methylpentan-2-yl)-4-oxo-1,4-dihydroquinoline-3-carboxylate (Intermediate 47, 0.24 g, 0.40 mmol) in MeOH (1 mL). The resulting solution was heated at reflux for one hour. The reaction mixture was cooled, diluted with water, acidified with 1N HCl, and then partitioned between EtOAc and water. The layers were separated and the aqueous layer was extracted with EtOAc. The organic layers were combined and washed with brine, then dried over sodium sulfate and concentrated under reduced pressure. The concentrate was purified by silica gel flash column chromatography (9:1 CH₂Cl₂/MeOH) to give 33 mg of the title compound as an off-white solid.

LC/MS (ES⁺)[(M+H)⁺]: 576 for C₃₀H₃₃N₅O₅S.

¹H NMR (300 MHz, d₆-DMSO): 0.4 (m, 2H), 0.7 (m, 2H), 0.88 (m, 6H), 1.11 (t, 3H), 1.47 (m, 1H), 1.86 (m, 1H), 1.94 (m, 2H), 3.21 (m, 2H), 3.79 (m, 2H), 5.22 (m, 2H), 7.35 (s, 1H), 7.68 (t, 1H), 7.78 (m, 1H), 8.06 (s, 1H), 8.25 (m, 3H), 8.9 (s, 1H), 9.39 (s, 1H), 15.12 (s, 1H).

### Intermediate 49

### (S)-6-(4-(4-cyclopropylthiazol-2-yl)-6-(3-ethylureido)pyridin-3-yl)-1-(1-(di-tert-butoxyphosphoryloxy)-4-methylpentan-2-yl)-4-oxo-1,4-dihydroquinoline-3-carboxylic acid

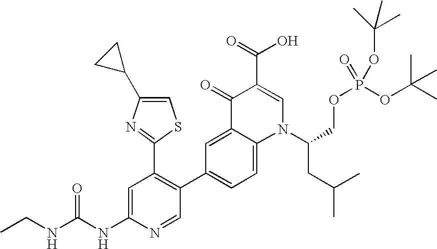

In a 25 mL round-bottom flask, (S)-6-(4-(4-cyclopropylthiazol-2-yl)-6-(3-ethylureido)pyridin-3-yl)-1-(1-hydroxy-4-methylpentan-2-yl)-4-oxo-1,4-dihydroquinoline-3-carboxylic acid (Intermediate 48, 0.32 g, 0.56 mmol) was dissolved in DMF (3 mL). 1H-tetrazole (2.471 mL, 1.11 mmol) and di-tert-butyl diethylphosphoramidite (0.356 mL, 1.28 mmol) were added. The reaction mixture was stirred at room temperature overnight. The reaction was cooled to −10° C. and H₂O₂ (0.122 mL, 1.20 mmol) was added. The reaction was warmed up to room temperature and left to stir for 1 h. The reaction was cooled to 0° C. and a solution of sodium metabisulfite (0.4 g) in water (1.5 ml) was added. The reaction was warmed to room temperature and stirred overnight. The reaction mixture was partitioned between EtOAc and water. The layers were separated and the aqueous layer was extracted with EtOAc. The organic layers were combined and washed with brine, then dried over sodium sulfate and concentrated under reduced pressure. The concentrate was purified by silica gel flash column chromatography (6% MeOH in CH₂Cl₂) to give 102 mg of the title compound as an off-white solid.

LC/MS (ES⁺)[(M+H)⁺]: 768 for C₃₈H₅₀N₅O₈PS.

### Intermediate 50

### 3-Aminopropyl dihydrogen phosphate

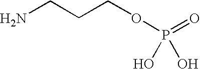

A solution of phosphorousoxychloride (13.71 mL, 146.6 mmol) in dichloromethane (120 mL) was cooled to 0° C. under nitrogen atmosphere. In another round bottomed flask, a solution 3-aminopropan-1-ol (10.0 g, 133.3 mmol), triethylamine (37.14 mL, 266.6 mmol) in dichloromethane (30 mL) was prepared and the solution was added slowly to the above reaction mixture over 30 min at 0° C. After the completion of the addition, the reaction mixture was slowly allowed to room temperature and stirred for 30 min. Then the reaction mixture was filtered and the filtrate was concentrated under reduced pressure to a residue. The residue was cooled to 0° C., then water (60 mL) and concentrated hydrochloric acid solution (0.6 mL) were added under constant stirring. The reaction mixture was refluxed for 2 h then cooled to room temperature and neutralized with triethylamine and stirred at room temperature for further 1 h. Ethanol (150 mL) was added slowly to the above mixture and the precipitated solid was filtered and dried to afford 8.0 g (38.4%) of 3-aminopropyl dihydrogen phosphate as white solid.

¹H NMR (400 MHz, D₂O): δ 1.22 (m, 1H), 1.95 (m, 2H), 3.13 (m, 2H), 3.95 (m, 2H).

### Intermediate 51

### Ethyl 7-bromo-1-[(5-methyl-1,2,4-oxadiazol-3-yl)methyl]-4-oxo-1,4-dihydroquinoline-3-carboxylate

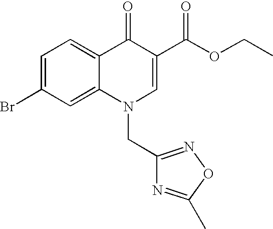

To a stirred solution of ethyl 7-bromo-4-oxo-1,4-dihydroquinoline-3-carboxylate (Intermediate 55, 3.5 g, 11.82 mmol) in dry dimethylformamide (40 mL), potassium carbonate (4.90 g, 85.47 mM) was added and stirred at room temperature. To the above reaction mixture 3-chloromethyl-5-methyl-1,2,4-oxadiazole (3.5 mL, 26.60 mmol) was added and the mixture was stirred at 80° C. for 2 h. After the completion of the reaction, the reaction mixture was filtered and the filtrate was concentrated to dryness. The residue was triturated with petroleum ether (40 mL) to obtain solid compound which was filtered and dried to afford 4.1 g (89.13%) of ethyl 7-bromo-1-[(5-methyl-1,2,4-oxadiazol-3-yl)methyl]-4-oxo-1,4-dihydroquinoline-3-carboxylate as pale brown solid.

¹H NMR (400 MHz, DMSO-d₆): δ 1.29 (t, 3H), 2.50 (s, 3H), 4.22-4.27 (q, 2H), 5.89 (s, 2H), 7.63-7.65 (m, 1H), 7.97 (d, 1H), 8.12-8.14 (d, 1H), 8.85 (s, 1H).

LC-MS: m/z 392.1 (M+H).

### Intermediate 52

### Ethyl 7-{6-[(ethylcarbamoyl)amino]-4-[4-(trifluoromethyl)-1,3-thiazol-2-yl]pyridin-3-yl}-1-[(5-methyl-1,2,4-oxadiazol-3-yl)methyl]-4-oxo-1,4-dihydroquinoline-3-carboxylate

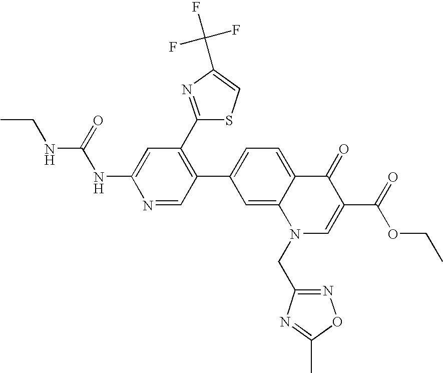

In a round bottomed flask ethyl 7-bromo-1-[(5-methyl-1,2,4-oxadiazol-3-yl)methyl]-4-oxo-1,4-dihydroquinoline-3-carboxylate (Intermediate 51, 1.0 g, 2.55 mmol), 1-ethyl-3-{5-(4,4,5,5-tetramethyl-1,3,2-dioxaborolan-2-yl)-4-[4-(trifluoromethyl)-1,3-thiazol-2-yl]pyridin-2-yl}urea (Intermediate 12A, 0.589 g, 0.51 mmol) and cesium carbonate (1.65 g, 5.10 mmol) were combined and suspended in dioxane:water (8:2). The above reaction mixture was purged with Argon gas for 30 min. Tetrakis (triphenylphosphine) palladium (0.589 g, 0.51 mM) was added under argon atmosphere and the reaction mixture was heated to 80-90° C. for 5 h. After completion of the reaction, the reaction mixture was cooled to room temperature, filtered through celite, and the filtrate was concentrated to dryness to give a residue. Water (30 mL) was added to the residue, and the water layer was extracted with ethyl acetate (2×50 mL). The organic layers were combined and dried over sodium sulfate then concentrated to give crude compound which was subjected to flash column chromatography (0-5% methanol/dichloromethane) to afford 0.5 g (33.3%) of ethyl 7-{6-[(ethylcarbamoyl)amino]-4-[4-(trifluoromethyl)-1,3-thiazol-2-yl]pyridin-3-yl}-1-[(5-methyl-1,2,4-oxadiazol-3-yl)methyl]-4-oxo-1,4-dihydroquinoline-3-carboxylate.

¹H NMR (400 MHz, DMSO-d₆): δ 1.10 (t, 3H), 1.30 (t, 3H), 3.19-3.22 (m, 2H), 4.23-4.29 (q, 2H), 5.82 (s, 2H), 7.32-7.42 (m, 2H), 7.54-7.59 (m, 2H), 7.73 (s, 1H), 8.24-8.27 (m, 2H), 8.43 (s, 1H), 8.88 (s, 1H), 9.43 (s, 1H).

LC-MS: m/z 627.1 (M+H).

### Intermediate 53

### 7-{6-[(ethylcarbamoyl)amino]-4-[4-(trifluoromethyl)-1,3-thiazol-2-yl]pyridin-3-yl}-1-[(5-methyl-1,2,4-oxadiazol-3-yl)methyl]-4-oxo-1,4-dihydroquinoline-3-carboxylic acid

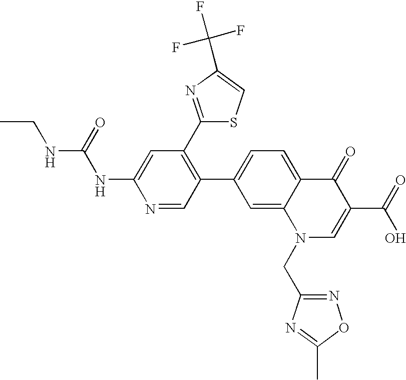

To a stirred solution of ethyl 7-{6-[(ethylcarbamoyl)amino]-4-[4-(trifluoromethyl)-1,3-thiazol-2-yl]pyridin-3-yl}-1-[(5-methyl-1,2,4-oxadiazol-3-yl)methyl]-4-oxo-1,4-dihydroquinoline-3-carboxylate (Intermediate 52, 0.5 g, 0.637 mmol) in methanol (10 mL) was added 10% potassium hydroxide (2.5 mL) and the mixture was heated to 80° C. for 1 h. After the completion of the reaction, the reaction mixture was concentrated to dryness to give a residue which was triturated with ethyl acetate (30 mL) and decanted. The residue was acidified to pH 2 with 2N hydrochloric acid solution (2 mL) to obtain a solid which was collected by filtration and dried. The solid was taken in methanol (10 mL) and stirred for 15 min. then filtered and dried to get 0.37 (79.07%) of 7-{6-[(ethylcarbamoyl)amino]-4-[4-(trifluoromethyl)-1,3-thiazol-2-yl]pyridin-3-yl}-1-[(5-methyl-1,2,4-oxadiazol-3-yl)methyl]-4-oxo-1,4-dihydroquinoline-3-carboxylic acid as colorless solid.

¹H NMR (400 MHz, DMSO-d₆): δ 1.10 (t, 3H), 3.17-3.21 (q, 2H) 6.05 (s, 2H), 7.32-7.42 (m, 2H), 7.57-7.59 (m, 2H), 7.98 (s, 2H), 8.25-8.31 (d, 1H), 8.39-8.45 (m, 2H), 9.27 (s, 1H), 9.47 (s, 1H).

LC-MS: m/z 600 (M+H).

### Intermediate 54

### Diethyl {[(3-bromophenyl)amino]methylidene} propanedioate

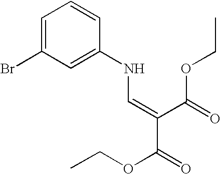

A mixture of 3-bromoaniline (10.0 g, 58.1 mmol) and diethyl (ethoxymethylidene)propanedioate (13.9 mL, 63.9 mmol) were heated to 110° C. for 1 h. After completion of the reaction, the reaction mixture was cooled to room temperature. The obtained solid was filtered and dried to afford 16.0 g 78% of diethyl {[(3-bromophenyl)amino]methylidene}propanedioate.

¹H NMR (400 MHz, CDCl₃): δ1.37 (m, 6H), 4.25 (m, 4H), 7.05 (d, 1H), 7.25 (m, 2H), 8.44 (d, 1H), 10.95 (d, 1H).

MASS (APCI+ve Scan): m/z 342.32 (M+H).

### Intermediate 55

### Ethyl 7-bromo-4-oxo-1,4-dihydroquinoline-3-carboxylate

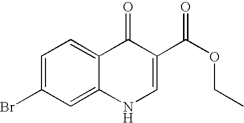

Dowtherm (50 mL) was taken in double neck round bottom flask (100 mL) and heated to 250° C. in sand bath. At this temperature diethyl {[(3-bromophenyl)amino]methylidene}propanedioate (Intermediate 54, 10.0 g, 29.2 mmol) was added and the mixture was maintained at 250° C. for another 1 h. After completion of the reaction, the reaction mixture was cooled to room temperature, and the solid was collected by filtration and dried to yield 6.0 g (69.3%) ethyl 7-bromo-4-oxo-1,4-dihydroquinoline-3-carboxylate as the predominant isomer.

¹H NMR (400 MHz, DMSO-d₆): δ 1.07 (t, 3H), 1.22 (s, 12H), 3.16 (q, 2H), 7.29 (m, 1H), 7.31 (d, 2H), 7.61 (s, 1H), 7.88 (s, 1H), 8.31 (s, 1H), 8.65 (s, 1H), 9.44 (s, 1H).

LC-MS: m/z 294 (M+H).

### Intermediate 56

### Ethyl 7-bromo-1-ethyl-4-oxo-1,4-dihydroquinoline-3-carboxylate

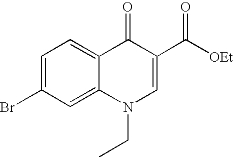

Ethyl 7-bromo-4-oxo-1,4-dihydroquinoline-3-carboxylate (Intermediate 55, 1.0 g, 3.3 mM) was dissolved in dimethylformamide (20 mL) and potassium carbonate (1.39 g, 10.1 mM) was added. After stirring the mixture for 10 min, ethyl bromide (0.76 mL, 10.1 mM) was added, and the reaction mixture was heated to 80° C. for 3 h. After completion of the reaction, the reaction mixture was cooled to room temperature, filtered through celite, and concentrated under reduced pressure to dryness. The residue was diluted with diethyl ether to obtain a solid which was filtered and dried to afford 750 mg (68.8%) of ethyl 7-bromo-1-ethyl-4-oxo-1,4-dihydroquinoline-3-carboxylate as white solid.

¹H NMR (400 MHz, DMSO-d₆):δ 1.26 (t, 3H), 1.34 (t, 3H), 4.21 (q, 2H), 4.42 (q, 2H), 7.64 (d, 1H), 8.06 (s, 1H), 8.25 (d, 1H), 8.68 (s, 1H).

LC-MS: m/z 324 (M+H).

### Intermediate 57

### Ethyl 1-ethyl-7-{6-[(ethylcarbamoyl)amino]-4-[4-(trifluoromethyl)-1,3-thiazol-2-yl]pyridin-3-yl}-4-oxo-1,4-dihydroquinoline-3-carboxylate

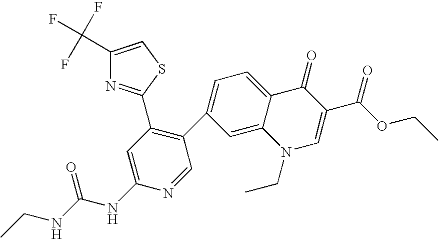

In a round bottomed flask ethyl 7-bromo-1-ethyl-4-oxo-1,4-dihydroquinoline-3-carboxylate (Intermediate 56, 200 mg, 0.61 mmol), 1-ethyl-3-{5-(4,4,5,5-tetramethyl-1,3,2-dioxaborolan-2-yl)-4-[4-(trifluoromethyl)-1,3-thiazol-2-yl]pyridin-2-yl}urea (Intermediate 12A, 32.7 mg, 0.74 mmol) and cesium carbonate (402 mg, 1.2 mmol) were combined and suspended in dioxane:water (8:2). Tetrakis (triphenylphosphine) palladium (142 mg, 0.2 mM) was added under argon atmosphere and the reaction mixture was heated to 80-90° C. for 2 h. After completion of the reaction, the reaction mixture was cooled to room temperature, filtered through celite, the organic solvent was concentrated under reduced pressure to give a residue. The residue was diluted with water and ethyl acetate to obtain solid which was filtered and dried to afford 140 mg (40.5%) of ethyl 1-ethyl-7-{6-[(ethylcarbamoyl)amino]-4-[4-(trifluoromethyl)-1,3-thiazol-2-yl]pyridin-3-yl}-4-oxo-1,4-dihydroquinoline-3-carboxylate.

¹H NMR (400 MHz, DMSO-d₆): δ 1.09 (t, 3H), 1.19 (t, 3H), 1.29 (t, 3H), 3.22 (q, 2H), 4.23 (q, 2H), 4.35 (q, 2H), 7.39 (m, 1H), 7.45 (d, 2H), 7.60 (m, 1H), 7.80 (s, 1H), 8.22 (s, 1H), 8.26 (d, 1H), 8.38 (s, 1H), 8.47 (s, 1H), 8.69 (s, 1H), 9.45 (s, 1H).

LC-MS: m/z 560 (M+H).

### Intermediate 58

### 1-ethyl-7-{6-[(ethylcarbamoyl)amino]-4-[4-(trifluoromethyl)-1,3-thiazol-2-yl]pyridin-3-yl}-4-oxo-1,4-dihydroquinoline-3-carboxylic acid

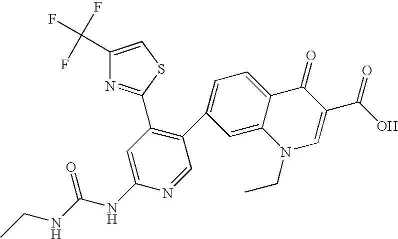

To a stirred suspension of ethyl 1-ethyl-7-{6-[(ethylcarbamoyl)amino]-4-[4-(trifluoromethyl)-1,3-thiazol-2-yl]pyridin-3-yl}-4-oxo-1,4-dihydroquinoline-3-carboxylate (Intermediate 57, 70 mg, 0.12 mmol) in methanol (5 mL) was added 10% potassium hydroxide (0.24 mL). The reaction mixture was heated to 60° C. for 1 h. After completion of the reaction, the reaction mixture was cooled to room temperature. The organic solvent was concentrated under reduced pressure to a residue. The residue was diluted with water and acidified with 2N HCl to pH 2-3 to obtain solid which was filtered, washed with water and dried to afford 1-ethyl-7-{6-[(ethylcarbamoyl)amino]-4-[4-(trifluoromethyl)-1,3-thiazol-2-yl]pyridin-3-yl}-4-oxo-1,4-dihydroquinoline-3-carboxylic acid 30 mg (44.7%).

¹H NMR (400 MHz DMSO-d₆): δ 1.11 (t, 3H), 1.24 (t, 3H), 3.22 (q, 2H), 4.57 (q, 2H), 7.60 (d, 1H), 8.05 (s, 1H), 8.22 (s, 1H), 8.40 (d, 2H), 8.49 (s, 1H), 9.08 (s, 1H), 9.49 (s, 1H).

LC-MS: m/z 532 (M+H).

### Intermediate 59

### Ethyl 7-bromo-1-(cyclopropylmethyl)-4-oxo-1,4-dihydroquinoline-3-carboxylate

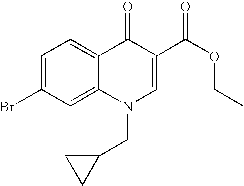

Intermediate 59 was prepared according to the procedure described for Intermediate 56 using Intermediate 55 and cyclopropylmethyl bromide as the starting material.

¹H NMR (400 MHz, CDCl₃): δ 0.48 (d, 2H), 0.79 (m, 2H), 1.39 (m, 1H), 1.42 (t, 3H), 3.98 (d, 2H), 4.42 (q, 2H), 7.53 (d, 1H), 7.65 (s, 1H), 8.40 (d, 1H), 8.54 (s, 1H).

LC-MS: m/z 350 (M+H).

### Intermediate 60

### Ethyl 1-(cyclopropylmethyl)-7-{6-[(ethylcarbamoyl)amino]-4-[4-(trifluoromethyl)-1,3-thiazol-2-yl]pyridin-3-yl}-4-oxo-1,4-dihydroquinoline-3-carboxylate

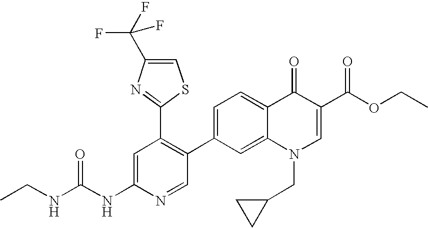

Intermediate 60 was prepared according to the procedure described for Intermediate 57 using Intermediate 59 and Intermediate 12A as the starting material.

¹H NMR (400 MHz, DMSO-d₆): δ 0.38 (m, 4H), 0.98 (m, 1H), 1.10 (t, 3H), 1.29 (t, 3H), 3.19 (q, 2H), 4.29 (q, 2H), 7.40 (d, 1H), 7.61 (s, 1H), 7.91 (s, 1H), 8.21 (s, 1H), 8.23 (d, 1H), 8.38 (s, 1H), 8.48 (s, 1H), 8.71 (s, 1H), 9.42 (s, 1H).

LC-MS: m/z 586 (M+H).

### Intermediate 61

### 1-(cyclopropylmethyl)-7-{6-[(ethylcarbamoyl)amino]-4-[4-(trifluoromethyl)-1,3-thiazol-2-yl]pyridin-3-yl}-4-oxo-1,4-dihydroquinoline-3-carboxylic acid

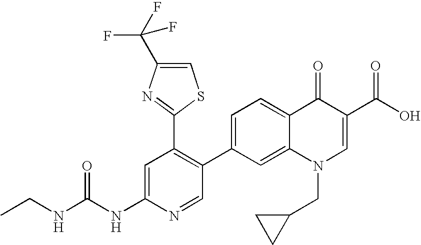

Intermediate 61 was prepared according to the procedure described for Intermediate 58 using Intermediate 60 as the starting material.

¹H NMR (400 MHz, DMSO-d₆): δ 0.44 (m, 4H), 1.09 (m, 1H), 1.11 (t, 3H), 3.22 (m, 3H), 4.42 (d, 2H), 7.61 (m, 2H), 8.13 (s, 1H), 8.21 (s, 1H), 8.40 (s, 1H), 8.42 (s, 1H), 9.08 (s, 1H), 9.50 (s, 1H).

LC-MS: m/z 558 (M+H).

### Intermediate 62

### Ethyl 7-bromo-1-(3-methylbutyl)-4-oxo-1,4-dihydroquinoline-3-carboxylate

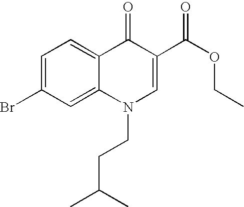

Intermediate 62 was prepared according to the procedure described for Intermediate 56 using Intermediate 55 and 3-methylbutylbromide as the starting material.

¹H NMR (400 MHz, CDCl₂): δ 1.04 (d, 6H), 1.41 (t, 3H), 1.78 (q, 3H), 4.14 (q, 2H), 4.40 (q, 2H), 7.52 (s, 1H), 7.56 (d, 1H), 8.39 (s, 1H), 8.42 (d, 1H).

### Intermediate 63

### Ethyl 7-{6-[(ethylcarbamoyl)amino]-4-[4-(trifluoromethyl)-1,3-thiazol-2-yl]pyridin-3-yl}-1-(3-methylbutyl)-4-oxo-1,4-dihydroquinoline-3-carboxylate

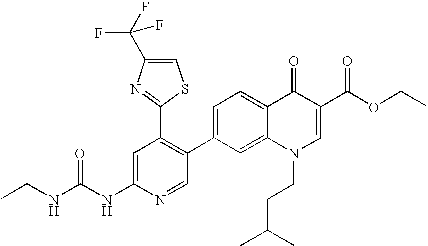

Intermediate 63 was prepared according to the procedure described for Intermediate 57 using Intermediate 62 and Intermediate 12A as the starting material.

¹H NMR (400 MHz, DMSO-d₆): δ 0.91 (d, 6H), 1.12 (t, 3H), 1.31 (t, 3H), 1.39 (d, 2H), 1.56 (m, 1H), 3.21 (q, 2H), 4.21 (q, 2H), 4.31 (t, 2H), 7.48 (d, 1H), 7.58 (t, 2H), 7.68 (s, 1H), 8.24 (s, 1H), 8.30 (d, 1H), 8.38 (s, 1H), 8.51 (s, 1H), 8.68 (s, 1H).

LC-MS: m/z 602 (M+H).

### Intermediate 64

### 7-{6-[(ethylcarbamoyl)amino]-4-[4-(trifluoromethyl)-1,3-thiazol-2-yl]pyridin-3-yl}-1-(3-methylbutyl)-4-oxo-1,4-dihydroquinoline-3-carboxylic acid

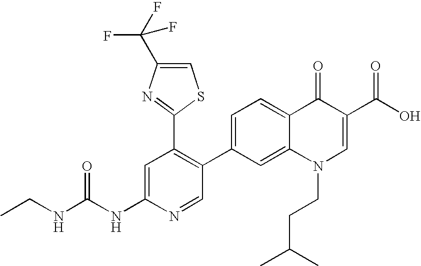

Intermediate 64 was prepared according to the procedure described for Intermediate 58 using Intermediate 63 as the starting material.

¹H NMR (400 MHz, DMSO-d₆): δ 0.83 (d, 6H), 1.13 (t, 3H), 1.44 (q, 2H), 1.55 (m, 1H), 3.21 (q, 2H), 4.52 (t, 2H), 7.56 (m, 1H), 7.63 (d, 1H), 7.93 (s, 1H), 8.25 (s, 1H), 8.40 (s, 1H), 8.43 (d, 1H), 8.51 (s, 1H), 9.06 (s, 1H), 9.48 (s, 1H).

LC-MS: m/z 574.2 (M+H).

### Intermediate 65

### Ethyl 7-bromo-4-oxo-1-propyl-1,4-dihydroquinoline-3-carboxylate

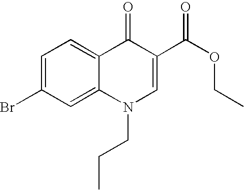

Intermediate 65 was prepared according to the procedure described for Intermediate 56 using Intermediate 55 and 3-bromopropane as the starting material.

¹H NMR (400 MHz, DMSO-d₆): δ 0.90 (t, 3H), 1.75 (q, 2H), 4.24 (q, 2H), 4.35 (q, 2H), 7.63 (d, 1H), 8.07 (s, 1H), 8.14 (d, 1H), 8.66 (s, 1H).

LC-MS: m/z 338 (M+H).

### Intermediate 66

### Ethyl 7-{6-[(ethylcarbamoyl)amino]-4-[4-(trifluoromethyl)-1,3-thiazol-2-yl]pyridin-3-yl}-4-oxo-1-propyl-1,4-dihydroquinoline-3-carboxylate

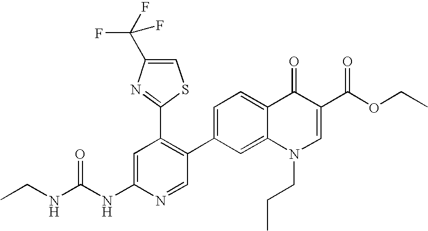

Intermediate 66 was prepared according to the procedure described for Intermediate 57 using Intermediate 65 and Intermediate 12A as the starting material.

¹H NMR (400 MHz, DMSO-d₆): δ 0.91 (t, 3H), 1.16 (t, 3H), 1.56 (m, 2H), 3.21 (q, 2H), 3.34 (s, 2H), 4.26 (q, 2H), 7.41 (d, 1H), 7.60 (s, 1H), 7.78 (s, 1H), 8.21 (s, 1H), 8.28 (d, 1H), 8.52 (s, 2H), 8.68 (s, 1H), 9.42 (s, 1H).

LC-MS: m/z 574 (M+H).

### Intermediate 67

### 7-{6-[ethylcarbamoyl)amino]-4-[4-(trifluoromethyl)-1,3-thiazol-2-yl]pyridin-3-yl}-4-oxo-1-propyl-1,4-dihydroquinoline-3-carboxylic acid

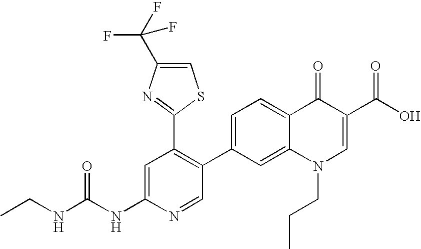

Intermediate 67 was prepared according to the procedure described for Intermediate 58 using Intermediate 66 as the starting material.

¹H NMR (400 MHz, DMSO-d₆): δ 0.79 (t, 3H), 1.11 (t, 3H), 1.55 (q, 2H), 3.21 (q, 2H), 4.48 (t, 2H), 7.60 (m, 2H), 8.00 (s, 1H), 8.21 (s, 1H), 8.41 (t, 2H), 8.51 (s, 1H), 9.06 (s, 1H), 9.49 (s, 1H).

LC-MS: m/z 546 (M+H).

### Intermediate 68

### di-tert-butyl hydrogen phosphate

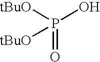

A solution of di-tert-butylphosphite (15.6 g, 80.8 mmol) and potassium hydrogencarbonate (4.84 g, 48.4 mmol) in deionized ultra-filtered (DIUF) water (70 mL) was cooled in an ice bath (see US20030195169). To this was added potassium permanganate (8.92 g, 56.5 mmol) in three equal portions over 1 h then the reaction was allowed to continue at room temperature for 30 min. Decolorizing carbon (−1 g) was added and the mixture heated to 60° C. for 15 min. The material was then cooled and filtered through Celite then washed with DIUF water to give a clear solution. If color persisted, the charcoal treatment was repeated. The solution was cooled in an ice bath and concentrated HCl was added (˜1 eq) slowly dropwise. The precipitate was collected by filtration and dried to give 7.2 g of white solid.

¹H NMR (300 MHz, CDCl₃): δ 1.48 (s, 18H), 11.4 (br s, 1H).

### Intermediate 69

### tetrabutyl ammonium di-tert-butyl phosphate

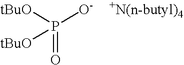

A solution of di-tert-butyl hydrogen phosphate (Intermediate 68, 7.2 g, 34.2 mmol) in acetone (60 mL) was cooled in an ice/salt bath to −5 to −10° C. To this was added dropwise a solution of tetrabutylammonium hydroxide (40% in water, 22.2 mL, 34.2 mmol). The mixture was stirred ˜30 min after addition then the solvents were removed by evaporation and the material was dried under vacuum to a semi-solid. The product was triturated with ether and filtered to give 13.6 g (37%) of product as a white solid.

¹H NMR (300 MHz, CDCl₃): δ 1.02 (t, 12H), 1.40 (s, 18H), 1.45 (m, 8H), 1.65 (m, 8H), 3.35 (m, 8H).

### Intermediate 70

### di-tert-butyl 2-hydroxyethyl phosphate

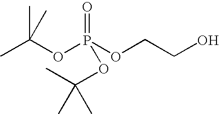

2-Bromoethanol was pretreated with potassium carbonate and filtered (syringe filter) before use. A solution of 2-bromoethanol (1.58 mL, 22.2 mmol) and tetrabutyl ammonium di-tert-butyl phosphate (Intermediate 69, 10 g, 22.2 mmol) in dimethoxyethane was heated at 80-85° C. for 3 h. The reaction was cooled and the solution diluted with 5-6 equivalents of ether. The solids were removed by filtration and the filtrate evaporated to give 6.6 g (>100%) of product as clear oil. The material was chromatographed on silica gel (20-100% ethyl acetate: heptanes) although it was not be entirely stable to prolonged exposure.

MS (ESP): 277.2 (M+Na⁺) for C₁₀H₂₃O₅P

¹H NMR (300 MHz, CDCl₃): δ 1.50 (s, 18H), 3.15 (br, 1H), 3.81 (m, 2H), 4.10 (m, 2H).

### Intermediate 71

### 2-(di-tert-butoxyphosphoryloxy)ethyl 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylate

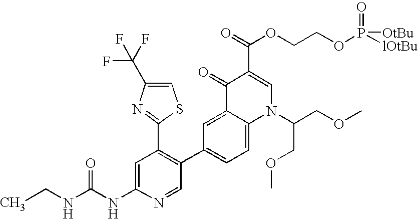

To a solution of 1-(1,3-dimethoxypropan-2-yl)-6-(6-(3-ethylureido)-4-(4-(trifluoromethyl)thiazol-2-yl)pyridin-3-yl)-4-oxo-1,4-dihydroquinoline-3-carboxylic acid (Intermediate 43, 3.49 g, 5.77 mmol), di-tert-butyl 2-hydroxyethyl phosphate (Intermediate 70, 1.75 g, 6.92 mmol) and triethyl amine (4 mL, 28.8 mmol) in dimethyl formamide (40 mL) was added diethyl cyanophosphonate (1 mL<6.92 mmol) and the mixture stirred at room temperature for 3 h. The reaction was incomplete so more diethyl cyanophosphonate (1 mL) was added and after stirring for several more hours no starting material remained. The mixture was diluted with water and ethyl acetate and the layers separated. The ethyl acetate layer was washed with water (3×) and the aqueous layer was backwashed with ethyl acetate. The combined organic layers were dried over sodium sulfate and the solvent was evaporated to give 9.4 g of crude reddish oil. The crude was chromatographed (2×) on an Analogix 115 g column (0-70% tetrahydrofuran: ethyl acetate) to remove most of the residual dimethyl formamide and some impurities followed the further purified on a C18, 55 g Analogix column in batches (dry loaded on Celite, elute 0-98% methanol in water) to give 1.2 g (25%) of the desired product (which had some trace impurities).

MS (ESP): 842 (M+H⁺) for C₃₇H₄₇P₃N₅O₁₀PS.

### Intermediates 72-76

The following Intermediates were prepared according to the procedure described for Intermediate 10 from the starting materials listed in the table.

| Int | Compound | Data | SM |
|---|---|---|---|
| 72 | 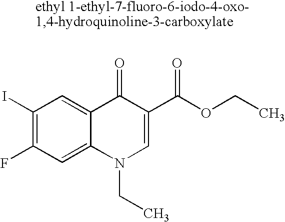 | Calcd for C₁₄H₁₃FINO₃ [M + H]⁺: 389.97 | Intermediate 83 and ethylamine |
| 73 | 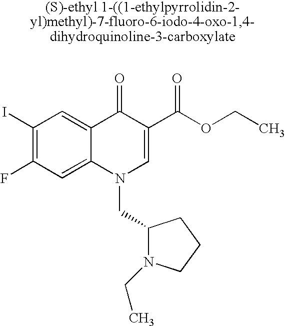 | Calcd for C₁₇H₁₈FIN₂O₃ [M + H]⁺: 472.98 | Intermediate 83 and (S)-(1-ethylpyrrolidin- 2-yl)methanamine |
| 74 | 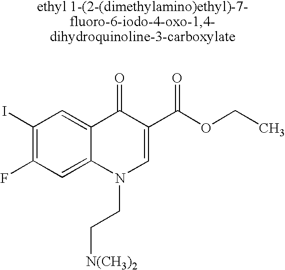 | Calcd for C₁₆H₁₈FIN₂O₃ [M + H]⁺: 432.99 | Intermediate 83 and N,N-dimethylethane- 1,2-diamine |
| 75 | 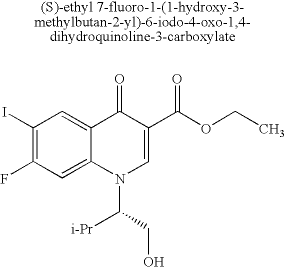 | Calcd for C₁₈H₂₁FINO₄ [M + H]⁺: 461.97. NMR (d₆-DMSO) δ 8.63 (d, 1 H), 8.58 (s, 1 H), 8.10 (d, 1H), 5.18-5.08 (m, 1 H), 4.93-4.85 (m, 1 H), 4.26-4.20 (m, 2 H), 3.71- 3.67 (m, 2 H), 1.92-1.68 (m, 2 H), 1.47-1.40 (m, 1 H), 1.27 (t, 3 H), 0.90 (d, 3 H), 0.85 (d, 3 H) | Intermediate 83 and (S)-2-amino-4- methylpentan-1-ol |
| 76 | 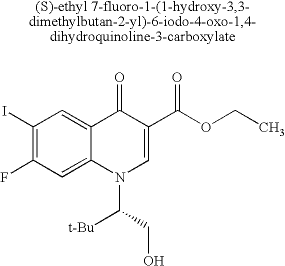 | NMR (d₆-DMSO) δ 8.64 (d, 1 H), 8.61 (s, 1 H), 8.24 (d, 1 H), 5.15-5.02 (m, 1 H), 4.83-4.78 (m, 1 H), 4.29-4.18 (m, 2 H), 3.99 (d, 2 H), 1.27 (t, 3 H), 0.96 (s, 9 H) | Intermediate 83 and (S)-2-amino-3,3- dimethylbutan-1-ol |

### Intermediate 77

### 1-ethyl-6-iodo-7-(4-methylpiperazin-1-yl)-4-oxo-1,4-dihydroquinoline-3-carboxylic acid

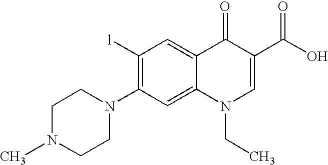

To a solution of 1-ethyl-7-fluoro-6-iodo-4-oxo-1,4-dihydroquinoline-3-carboxylic acid (Intermediate 78, 122 mg, 0.34 mmol, 1 equiv.) in DMF (1.5 mL) was added 1-methylpiperazine (75 μL, 0.34 mmol, 1 equiv.) followed by triethylamine (0.14 mL, 1.01 mmol, 3 equiv.). The reaction mixture was stirred at 70° C. for 8 h. The reaction mixture was cooled to room temperature, quenched with water (0.5 mL), and concentrated under reduced pressure. The resulting solid was purified (silica gel chromatography) and concentrated under reduced pressure to provide 1-ethyl-6-iodo-7-(4-methylpiperazin-1-yl)-4-oxo-1,4-dihydroquinoline-3-carboxylic acid as a solid (149 mg, >99%).

C₁₇H₂₀IN₃O₃ [M+H]⁺: 441.91.

### Intermediate 78

### 1-ethyl-7-fluoro-6-iodo-4-oxo-1,4-dihydroquinoline-3-carboxylic acid

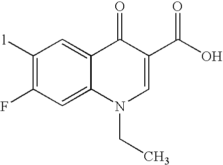

To a solution of ethyl 1-ethyl-7-fluoro-6-iodo-4-oxo-1,4-dihydroquinoline-3-carboxylate (Intermediate 72, 1.18 g, 3.03 mmol, 1 equiv.) in ethanol (12 mL) was added 2 N sodium hydroxide (3 mL, 6.06 mmol, 2 equiv.). The reaction mixture was stirred at 60° C. for 15 min. The reaction mixture was cooled to room temperature. 1 N HCl was added until pH 3-4 was reached. The precipitate was washed with ethanol and dried to provide 1-ethyl-7-fluoro-6-iodo-4-oxo-1,4-dihydroquinoline-3-carboxylic acid as a solid (1.05 g, 96%).

C₁₂H₉FINO₃ [M+H]⁺: 361.90.

### Intermediates 79-80

The following Intermediates were prepared according to the procedure described for Intermediate 78 from the starting materials indicated in the table.

| Int | Compound | Data | SM |
|---|---|---|---|
| 79 | 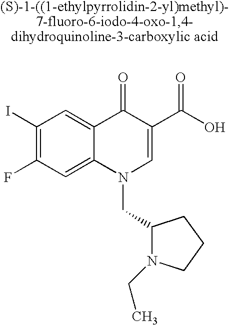 | Calcd for C₁₇H₁₈FIN₂O₃ [M + H]⁺: 444.98 | Intermediate 73 |
| 80 | 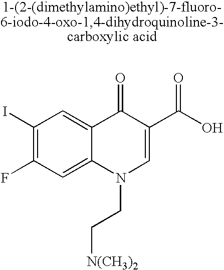 | Calcd for C₁₈H₁₄FIN₂O₃ [M + H]⁺: 404.95 | Intermediate 74 |

### Intermediate 81

### (S)-7-(2-(dimethylamino)ethoxy)-1-((1-ethylpyrrolidin-2-yl)methyl)-6-iodo-4-oxo-1,4-dihydroquinoline-3-carboxylic acid

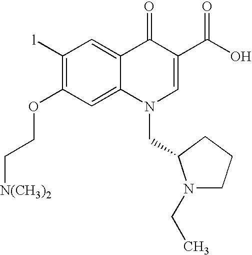

To a suspension of sodium hydride (279 mg, 6.98 mmol, 3 equiv.) in THF (12 mL) cooled to 0° C. was added 2-(dimethylamino)ethanol (0.247 mL, 2.46 mmol, 1.05 equiv.). This reaction mixture was stirred for 5 min. (S)-1-((1-ethylpyrrolidin-2-yl)methyl)-7-fluoro-6-iodo-4-oxo-1,4-dihydroquinoline-3-carboxylic acid (Intermediate 79, 1.03 g, 2.33 mmol, 1 equiv.) was subsequently added. This was warmed up to 40° C. and stirred for 8 h. The reaction was cooled to 0° C. and quenched with saturated ammonium chloride (2 mL). The reaction was partitioned between water (20 mL) and dichloromethane (20 mL). The aqueous layer was extracted with dichloromethane (2×20 mL), and the organics were concentrated. The compound was purified (silica gel chromatography) and concentrated to provide (S)-7-(2-(dimethylamino)ethoxy)-1-((1-ethylpyrrolidin-2-yl)methyl)-6-iodo-4-oxo-1,4-dihydroquinoline-3-carboxylic acid as a solid (1.2 g, >99%).

Calcd for C₂₁H₂₈IN₃O₄ [M+H]⁺: 514.03.

NMR (d₆-DMSO) δ 15.12 (s, 1H), 8.85 (s, 1H), 8.68 (s, 1H), 7.37 (s, 1H), 4.64-4.59 (dd, 1H), 4.45-4.29 (m, 3H), 3.08-2.99 (m, 2H), 2.78 (t, 2H), 2.30 (s, 6H), 2.20-2.09 (m, 3H), 1.84-1.79 (m, 1H), 1.72-1.67 (m, 2H), 1.53-1.46 (m, 1H), 0.73 (t, 3H).

### Intermediate 82

The following Intermediate was prepared according to the procedure described for Intermediate 81 from the starting materials described in the table.

| Int | Compound | Data | SM |
|---|---|---|---|
| 82 | 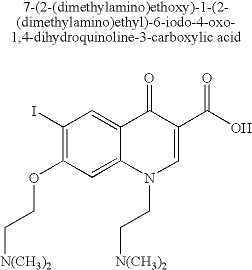 | Calcd for C₁₈H₂₄IN₃O₄ [M + H]⁺: 474.01 | Intermediate 81 |

### Intermediate 83

The following Intermediate was prepared according to the procedure described for Intermediate 11 from the starting material listed in the table.

| Int | Compound | Data | SM |
|---|---|---|---|
| 83 | 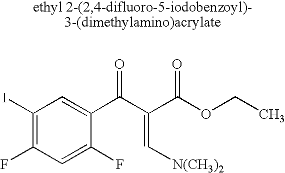 | Calcd for C₁₄H₁₄F₂INO₃ [M + H]⁺: 410.02. H¹NMR (d₆-DMSO) δ 7.82 (t, 1 H), 7.77 (s, 1 H), 7.34 (dd, 1 H), 3.93-3.86 (m, 2 H), 3.29 (s, 3 H), 2.78 (s, 3 H), 0.92 (t, 3 H) | Intermediate 84 |

### Intermediate 84

### 2,4-difluoro-5-iodobenzoic acid

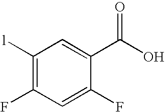

To a solution of 2,4-difluorobenzoic acid (25 g, 158.13 mmol, 1 equiv.) in sulfuric acid (315 mL) cooled to 0° C. was added N-iodosuccinimide (35.6 g, 158.13 mmol, 1 equiv.) portion-wise. The reaction was stirred at 0° C. for 4 h. The reaction was warmed up to room temperature and poured onto water (600 mL). 1 N sodium thiosulfate (200 mL) was added. The precipitate was washed with water and dried. The compound was dissolved in hot 1:1 ethanol:water. The yellow precipitate was discarded. Additional water was added, and the white precipitate was collected and dried to provide 2,4-difluoro-5-iodobenzoic acid as a solid (36 g, 80%).

Calcd for C₇H₃F₂IO₂ [M+H]⁺: 284.92.

NMR (d₆-DMSO) δ 13.51 (s, 1H), 8.26 (t, 1H), 7.51-7.44 (m, 1H).

### Intermediate 85

### Ethyl 1-[2-(dimethylamino)ethyl]-6-iodo-4-oxo-1,4-dihydroquinoline-3-carboxylate

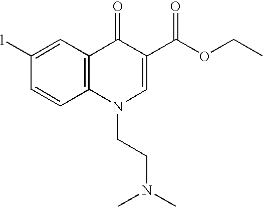

To a stirred solution of ethyl 3-(2-fluoro-5-iodophenyl)-3-oxopropanoate (Intermediate 102) (5 g, 14.92 mmol) in acetic anhydride (4.22 g, 44.77 mmol) was added methylorthoformate (4.8 g, 44.77 mmol) and heated to 140° C. for 16 h. After the completion of the reaction, excess acetic anhydride was concentrated to yield crude ethyl (2E)-2-[(2-fluoro-5-iodophenyl)carbonyl]-3-methoxyprop-2-enoate. This was dissolved in dichloromethane (100 mL), and N,N-dimethyl ethylamine (3.28 mL, 29.84 mmol) was added and stirred at room 1 h. After the completion of the reaction, the reaction mixture was quenched with water (200 mL). The organic layer was separated, dried and concentrated to yield crude ethyl (2E)-3-{[2-(dimethylamino)ethyl]amino}-2-[(2-fluoro-5-iodophenyl)carbonyl]prop-2-enoate, which was dissolved in acetonitrile (100 mL) and potassium carbonate (4.05 g, 29.84 mmol) was added. The reaction mixture was refluxed at 80° C. for 5 h. After the completion of the reaction, the reaction mixture was concentrated to dryness. Water (100 mL) was added and the reaction was stirred for 10 min. The precipitated solid was filtered and dried to obtain ethyl 1-[2-(dimethylamino)ethyl]-6-iodo-4-oxo-1,4-dihydroquinoline-3-carboxylate 3.3 g (53.27%)

¹H NMR (400 MHz, DMSO-d₆): δ 1.21 (t, 3H), 2.19 (s, 6H), 2.58 (t, 2H), 4.21 (q, 2H), 4.41 (t, 2H), 7.61 (d, 1H), 8.06 (d, 1H), 8.49 (s, 1H), 8.61 (d, 1H).

LC-MS: m/z 414(M+H)

### Intermediate 86-89

The following Intermediate was prepared according to the procedure described for Intermediate 85 from the starting material listed in the table.

| Int | Compound | Data | SM |
|---|---|---|---|
| 86 | 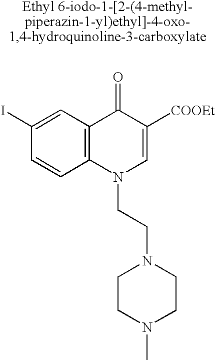 | ¹H NMR (400 MHz, DMSO-d₆): δ 1.26 (t, 3 H), 2.10 (s, 3 H), 2.21 (m, 4 H), 2.42 (m, 4 H), 2.60 (t, 2 H), 4.21 (q, 2 H), 4.41 (t, 2 H), 7.66 (d, 1 H), 8.60 (d, 1 H), 8.51 (m, 2 H). LC-MS: m/z 469 (M + H) | Intermediate 102 and 2-(4-methylpiperazin- 1-yl)ethanamine |
| 87 | 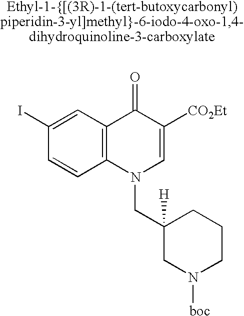 | ¹H NMR (400 MHz, DMSO-d₆): δ 1.41 (m, 15 H), 1.81 (m, 3 H), 2.61 (m, 2 H), 3.76 (m, 2 H), 4.21 (m, 3 H), 7.71 (d, 1 H), 8.02 (d, 1 H), 8.48 (s, 1 H), 8.68 (d, 1 H). LC-MS: m/z 540 (M + H) | Intermediate 102 and (R)-tert-butyl 3- (aminomethyl)piperidine- 1-carboxylate |
| 88 | 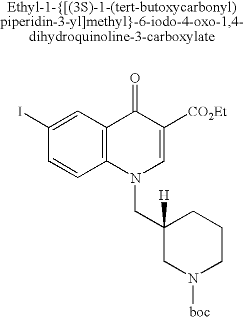 | ¹H NMR (400 MHz, DMSO-d₆): δ 1.41 (m, 9 H), 1.62 (m, 6 H), 2.16 (m, 1 H), 2.81 (s, 1 H), 2.98 (m, 1 H), 3.98 (m, 4 H), 4.18 (s, 1 H), 4.21 (q, 2 H), 7.18 (d, 1 H), 7.98 (d, 1 H), 8.41 (d, 1 H), 8.84 (s, 1 H). LC-MS: m/z 540 (M + H) | Intermediate 102 and (S)-tert-butyl 3- (aminomethyl)piperidine- 1-carboxylate |
| 89 | 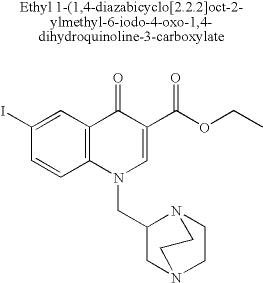 | ¹H NMR (400 MHz, DMSO-d₆): δ 1.27 (t, 3 H), 2.25 (m, 1 H), 2.44 (m, 2 H), 2.63 (m, 5 H), 2.94 (m, 2 H), 3.09 (m, 1 H), 4.22 (q, 2 H), 4.40 (m, 2 H), 7.69 (d, 1 H), 8.05 (d, 1 H), 8.54 (m, 2 H). LC-MS: m/z 468.3 (M + H) | Intermediate 102 and 1,4-diazabicyclo[2.2.2] octan-2-ylmethanamine |

### Intermediate 90

1-[2-(Dimethylamino)ethyl]-6-iodo-4-oxo-1,4-dihydroquinoline-3-carboxylic acid

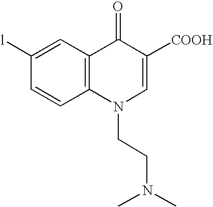

To a stirred solution of ethyl 1-[2-(dimethylamino)ethyl]-6-iodo-4-oxo-1,4-dihydroquinoline-3-carboxylate (Intermediate 85) (2 g, 4.83 mM) in methanol (50 mL) was added 10% potassium hydroxide solution (9.66 mL, 17.14 mM). The above reaction mixture was heated to 70° C. for 1 h. After the completion of the reaction, the reaction mixture was concentrated to dryness. The residue was diluted with water (50 mL). The aqueous layer was extracted with diethyl ether (2×80 mL) to remove organic impurities, if any, and was discarded. The aqueous layer was acidified to pH 2 using 2N hydrochloric acid solution (12 mL). The precipitated solid was filtered and dried to obtain 1.4 g (75.26%) of 1-[2-(dimethylamino)ethyl]-6-iodo-4-oxo-1,4-dihydroquinoline-3-carboxylic acid (Intermediate 90).

LC-MS: m/z 386 (M+H)

### Intermediate 91-94

The following Intermediates were prepared according to the procedure described for Intermediate 90 from the starting material listed in the table.

| Int | Compound | Data | SM |
|---|---|---|---|
| 91 | 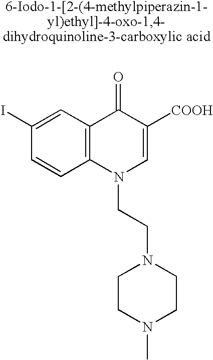 | ¹H NMR (400 MHz, DMSO-d₆): δ 2.42 (m, 2 H), 2.72 (m, 3 H), 2.81 (m, 4 H), 3.02 (d, 2 H) 3.33 (d, 2 H), 4.61 (s, 2 H), 7.82 (d, 1 H), 8.23 (d, 1 H), 8.62 (s, 1 H), 9.00 (s, 1 H), 10.55 (s, 1 H). LC-MS: m/z 441 (M + H) | Intermediate 86 |
| 92 | 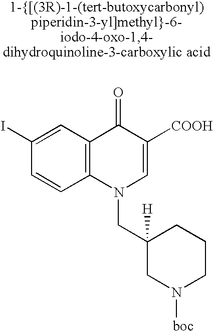 | ¹H NMR (400 MHz, DMSO-d₆): δ 1.21 (m, 11 H), 1.61 (s, 2 H), 1.96 (s, 1 H) 2.65 (m, 2 H), 3.78 (d, 1 H), 4.57 (s, 2 H), 7.95 (d, 1 H), 8.21 (d, 1 H), 8.61 (s, 1 H), 9.02 (d, 1 H), 14.85 (s, 1 H). LC-MS: m/z 513 (M + H) | Intermediate 87 |
| 93 | 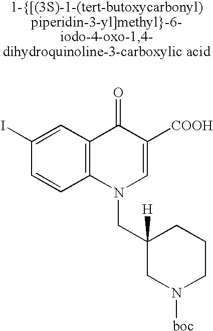 | ¹H NMR (400 MHz, DMSO-d₆): δ 1.39 (s, 11 H), 1.73 (m, 6 H), 2.12 (s, 1 H), 2.81 (m, 1 H), 2.98 (t, 4 H), 3.81 (m, 4 H), 4.01 (s, 2 H), 4.38 (s, 1 H), 7.38 (d, 1 H), 8.11 (d, 1 H), 8.62 (s, 1 H), 8.92 (s, 1 H), 14.58 (s, 1 H). LC-MS: m/z 513 (M + H) | Intermediate 88 |
| 94 | 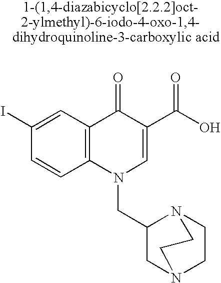 | ¹H NMR (400 MHz, DMSO-d₆): δ, 2.76-2.99 (m, 9 H), 3.30 (m, 2 H), 4.69 (m, 2 H), 7.94 (d, 1 H), 8.23 (d, 1 H), 8.64 (s, 1 H), 8.89 (s, 1 H), 14.9 (brs, 1 H). LC-MS: m/z 440.34 (M + H) | Intermediate 89 |

### Intermediate 95

### 1-[2-(Dimethylamino)ethyl]-6-iodo-N,N-dimethyl-4-oxo-1,4-dihydroquinoline-3-carboxamide

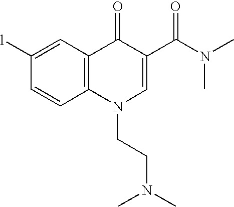

To a stirred solution of 1-[2-(dimethylamino)ethyl]-6-iodo-4-oxo-1,4-dihydroquinoline-3-carboxylic acid (Intermediate 90) (0.5 g, 1.295 mmol) in N,N-dimethyl formamide (20 mL), were added triethylamine (0.36 mL, 2.59 mmol) and HATU (0.738 g, 1.94 mmol). The reaction mixture was stirred at room temperature for 1 h. The reaction mixture was saturated with N, N-dimethyl amine gas at −20° C. The above reaction mixture was heated at 80° C. for 8 h. After the completion of the reaction, the reaction mixture was concentrated and purified by flash column chromatography (0-10% methanol in chloroform containing 2% triethylamine) to obtain 0.5 g (93.63%) of 1-[2-(dimethylamino)ethyl]-6-iodo-N,N-dimethyl-4-oxo-1,4-dihydroquinoline-3-carboxamide (Intermediate 95).

¹H NMR (400 MHz, DMSO-d₆): δ 2.61 (s, 6H), 2.81 (m, 5H), 2.98 (s, 2H), 3.21 (s, 2H), 4.61 (s, 2H), 7.79 (s, 1H), 8.22 (s, 1H), 8.51 (s, 1H), 8.81 (s, 1H).

LC-MS: m/z 413(M+H) in APCI mode.

### Intermediate 96-94

The following Intermediates were prepared according to the procedure described for Intermediate 95 from the starting material listed in the table.

| Int | Compound | Data | SM |
|---|---|---|---|
| 96 | 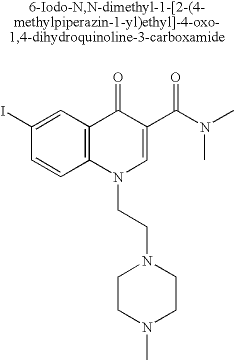 | ¹H NMR (400 MHz, DMSO-d₆): δ 2.62 (m, 9 H), 2.85 (m, 7 H), 3.24 (m, 3 H), 4.39 (s, 2 H), 7.62 (s, 1 H), 8.41 (m, 2 H), 8.95 (m, 1 H). LC-MS: m/z 468 (M + H) | Intermediate 91, HATU and N, N-dimethyl amine |
| 97 | 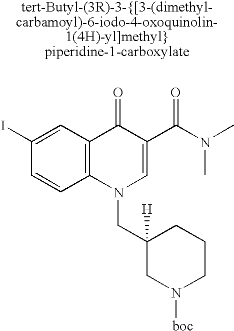 | ¹H NMR (400 MHz, DMSO-d₆): δ 1.21 (m, 10 H), 2.61 (m, 3 H), 2.98 (s, 3 H), 3.39 (m, 1 H), 3.79 (m, 1 H), 4.21 (s, 2 H), 7.62 (d, 1 H), 8.10 (d, 1 H), 8.21 (s, 1 H), 8.51 (s, 1 H), 14.85 (s, 1 H). LC-MS: m/z 540 (M + H) | Intermediate 92, HATU and N, N-dimethyl amine |
| 98 | 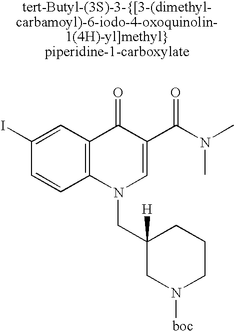 | ¹H NMR (400 MHz, DMSO-d₆): δ 1.41 (s, 11 H), 1.69 (m, 2 H), 2.11 (s, 1 H), 2.71 (m, 1 H), 2.99 (m, 3 H), 3.10 (m, 5 H), 3.73 (m, 3 H), 4.08 (s, 1 H), 7.19 (d, 1 H), 7.98 (m, 2 H), 8.78 (s, 1 H). LC-MS: m/z 540 (M + H) | Intermediate 93, HATU and N, N-dimethyl amine |
| 99 | 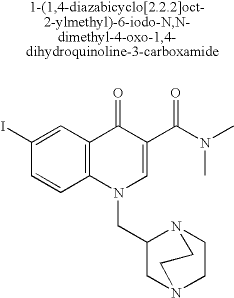 | ¹H NMR (400 MHz, DMSO-d₆): δ 2.25 (m, 2 H), 2.62 (m, 4 H), 2.95 (m, 6 H), 3.34 (m, 6 H), 4.34 (m, 2 H), 7.65 (d, 1 H), 8.03-8.10 (m, 2 H), 8.49 (s, 1 H). LC-MS: m/z 467.3 (M + H) | Intermediate 94, HATU and N, N-dimethyl amine |

### Intermediate 100-101

The following Intermediates were prepared according to the procedure described for Example 29 from the starting material listed in the table.

| Int | Compound | Data | SM |
|---|---|---|---|
| 100 | 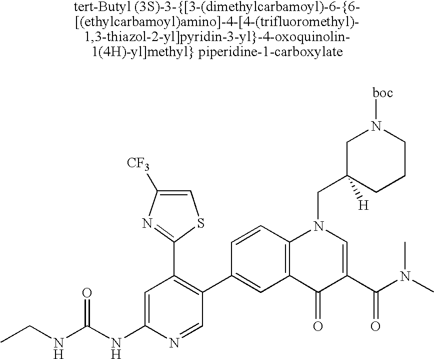 | ¹H NMR (400 MHz, DMSO-d₆): δ 1.11 (t, 3 H), 1.21 (s, 9 H), 1.61 (m, 2 H), 1.92 (s, 1 H), 2.62 (m, 2 H), 2.62 (s, 3 H), 2.82 (s, 3 H), 2.98 (s, 2 H), 3.21 (q, 2 H), 3.32 (m, 3 H), 3.37 (m, 1 H), 4.12 (q, 2 H), 7.61 (m, 2 H), 7.88 (d, 1 H), 8.18 (s, 2 H), 8.22 (s, 2 H), 8.48 (s, 1 H), 9.42 (s, 1 H). LC-MS: m/z 728 (M + H) | Intermediate 97 and 12. Pd(PPh3)4, Cs2CO3 |
| 101 | 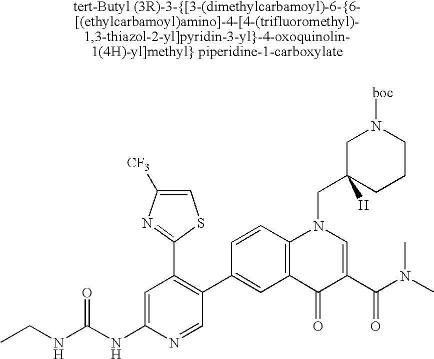 | ¹H NMR (400 MHz, DMSO-d₆): δ 1.18 (t, 3 H), 1.21 (s, 9 H), 1.62 (m, 2 H), 1.96 (s, 1 H), 2.68 (m, 2 H), 2.88 (m, 4 H), 2.98 (s, 3 H), 3.21 (q, 2 H), 3.78 (m, 1 H), 4.21 (q, 2 H), 7.61 (m, 2 H), 7.88 (d, 1 H), 8.14 (s, 1 H), 8.21 (s, 2 H), 8.34 (s, 1 H), 8.48 (s, 1 H), 9.42 (s, 1 H). LC-MS: m/z 728 (M + H) | Intermediate 98 and 12. Pd(PPh3)4, Cs2CO3 |

### Intermediate 102

### Ethyl 3-(2-fluoro-5-iodophenyl)-3-oxopropanoate

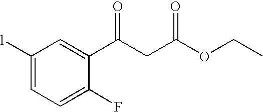

To a solution of 2-fluoro-5-iodobenzoic acid (200 g, 751.8 mmol, Aldrich) in dichloromethane (500 mL) thionyl chloride (500 mL) was added and refluxed for 3 h. Excess distilled thionyl chloride and dichloromethane were evaporated under reduce pressure to yield 2-fluoro-5-iodobenzoyl chloride, which was then dissolved in dry tetrahydrofuran (200 mL). Meanwhile, in another round bottom flask ethyl hydrogen malonoate (198 g, 1503 mmol) was dissolved in tetrahydrofuran (2 L) and cooled to 10° C. To this solution triethylamine (531 g, 5260 mmol) followed by magnesium chloride (285 g, 3000 mmol) were added and stirred for 6 h at room temperature To this reaction mixture the above acid chloride mixture was added at 0° C. stirred for 12 h at room temperature. After the completion of the reaction, the reaction mixture was acidified (3-4 pH) with 2 N hydrochloric acid and extracted with ethyl acetate (3×3L). The organic layers were combined and dried over anhydrous sodium sulphate, filtered and concentrated under reduced pressure to get crude compound, which was purified by flash column chromatography (0-2% ethyl acetate/pet ether) to afford 100 g (39%) of ethyl 3-(2-fluoro-5-iodophenyl)-3-oxopropanoate (Intermediate 102).

Mass (APCI+ve Scan): m/z 337 (M+H).

¹H NMR (400 MHz, CDCl₃): δ 1.23 (t, 3H), 3.99 (s, 2H), 4.23 (q, 2H), 6.93-6.97 (q, 1H), 7.83-7.85 (d, 1H), 8.2 (d, 1H).

### Intermediate 103-104

The following Intermediates were prepared according to the procedure described for Intermediate 85 from the starting material listed in the table.

| Int | Compound | Data | SM |
|---|---|---|---|
| 103 | 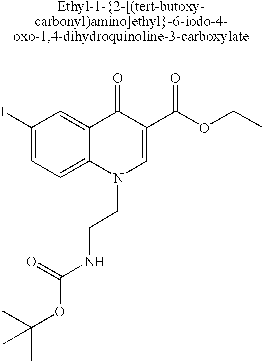 | ¹H NMR (400 MHz, DMSO-d₆): δ 1.24 (s, 9 H), 1.25-1.29 (t, 3 H), 3.34-3.74 (m, 2 H), 4.19-4.24 (q, 2 H), 4.35 (m, 2 H), 7.03 (s, 1 H), 7.66 (d, 1 h), 8.05 (d, 1 H), 8.48 (s, 2 H), Mass (APCI +ve Scan): m/z 487 (M + H). | Intermediate 102 and tert-butyl (2- aminoethyl)carbamate |
| 104 | 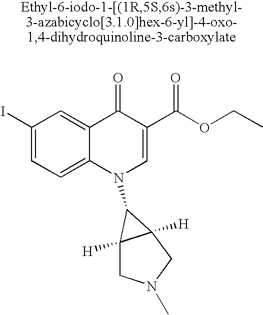 | ¹H NMR (400 MHz, DMSO-d₆): δ 1.28 (m, 2 H), 2.16 (s, 2 H), 2.26 (s, 3 H), 2.24 (m, 2 H), 3.27 (m, 2 H), 3.74 (m, 1 H), 4.20 (m, 2 H), 8.01 (m, 2 H), 8.51 (m, 2 H). LC-MS: m/z 439.33 (M + H) | Intermediate 102 and 110 |

### Intermediate 103-104

The following Intermediates were prepared according to the procedure described for Example 29 from the starting material listed in the table.

| Int | Compound | Data | SM |
|---|---|---|---|
| 105 | Ethyl 1-{2-[(tert- butoxycarbonyl)amino]ethyl}-6-{6- [(ethylcarbamoyl)amino]-4-[4- (trifluoromethyl)-1,3-thiazol-2- yl]pyridin-3-yl}-4-oxo-1,4- dihydroquinoline-3-carboxylate 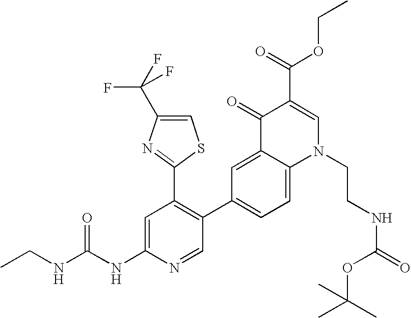 | ¹H NMR (400 MHz, DMSO- d₆): 1.11 (t, 6H), 1.28 (s, 9H), 3.22 (q, 2H), 3.43 (b, 2H), 4.21 (q, 2H), 4.41 (m, 2H), 7.12 (s, 1H), 7.65-7.79 (d, 2H), 7.95 (d, 1H), 8.15 (s, 1H), 8.26 (s, 1H), 8.35 (s, 1H), 8.48-8.52 (s, 2H), 9.52 (s, 1H). LC-MS: m/z 661 (M + H) | Intermediate 103 and 12. Pd(PPh3)4, Cs2CO3 |
| 106 | ethyl 6-{6-[(ethylcarbamoyl)amino]- 4-[4-(trifluoromethyl)-1,3-thiazol-2- yl]pyridin-3-yl}-1-[(1R,5S,6s)-3- methyl-3-azabicyclo[3.1.0]hex-6-yl]- 4-oxo-1,4-dihydroquinoline-3-carboxylate 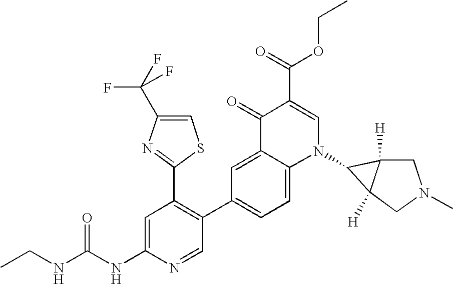 | ¹H NMR (400 MHz, DMSO- d₆): δ 1.16 (m, 3H), 1.28 (m, 3H), 2.30 (m, 5H), 2.45 (m, 2H), 3.18 (,. 3H), 3.76 (m, 2H), 4.21 (m, 2H), 7.60 (m, 2H), 7.98 (d, 1H), 8.11 (s, 1H), 8.24 (s, 1H), 8.30 (d, 1H), 8.56 (m, 2H), 9.45 (s, 1H) LC-MS: m/z 627.53 (M + H) | Intermediate 104 and 12. Pd(PPh3)4, Cs2CO3 |

### Intermediate 107

The following Intermediates were prepared according to the procedure described for Example 35 from the starting material listed in the table.

| Int | Compound | Data | SM |
|---|---|---|---|
| 107 | 1-{2-[(Tert- butoxycarbonyl)amino]ethyl}-6-{6- [(ethylcarbamoyl)amino]-4-[4- (trifluoromethyl)-1,3-thiazol-2- yl]pyridin-3-yl}-4-oxo-1,4- dihydroquinoline-3-carboxylic acid 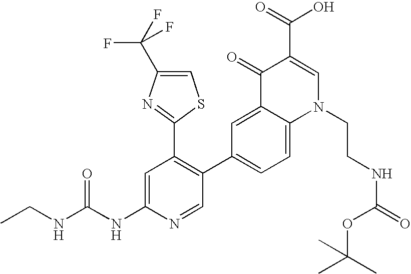 | ¹H NMR (400 MHz, DMSO- d₆): 1.09 (t, 3H), 1.23 (s, 9H), 3.31 (m, 2H), 3.37 (q, 2H), 4.60 (m, 2H), 7.08 (s, 1H), 7.63 (s, 1H), 7.90 (d, 1H), 8.15 (d, 1H), 8.25 (s, 1H), 8.31 (s, 1H), 8.39 (s, 1H), 8.49 (s, 1H), 8.88 (s, 1H), 9.49 (s, 1H). LC-MS: m/z 647 (M + H), (M + 1) | Intermediate 105 |

### Intermediate 108

### (1R,5S,6r)-3-methyl-6-nitro-3-azabicyclo[3.1.0]hexane-2,4-dione

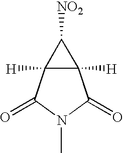

To a stirred solution of 1-methyl-1H-pyrrole-2,5-dione (10 g, 90 mmol) in acetonitrile (150 mL) was added potassium carbonate (12.42 g, 90 mmol) at room temperature and stirred for 15 min, bromonitromethane (12.6 g, 90 mmol) was added slowly at room temperature. Bromonitromethane (2.5 g, 18 mmol) was added for every 4 h for 3 times. The progress of the reaction was monitored by TLC. After completion of the reaction, the reaction mixture was passed through a bed of Celite and washed with acetonitrile. The filtrate was concentrated under reduced pressure, and purified by silica gel flash column chromatography (100-200 mesh) (10-20% ethyl acetate/petroleum ether). Isolation gave 3.5 g of the title compound (1R,5S, 6r)-3-methyl-6-nitro-3-azabicyclo [3.1.0] hexane-2,4-dione (Intermediate 108) as pale yellow solid in a 23% yield.

¹H NMR (400 MHz, CDCl₃): δ 2.92 (s, 3H), 3.38 (s, 2H), 4.62 (d, 1H).

MASS (APCI−ve Scan) m/z 169 (M−H)

### Intermediate 109

### (1R,5S,6s)-3-methyl-6-nitro-3-azabicyclo[3.1.0]hexane

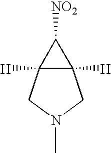

To a stirred solution of (1R,5S, 6r)-3-methyl-6-nitro-3-azabicyclo [3.1.0] hexane-2,4-dione (Intermediate 108) (3.5 g, 20.58 mmol) in tetrahydrofuran was added sodium borohydride (2.07 g, 55.58 mmol) portion-wise at room temperature for about 20 min. The reaction mixture was stirred at room temperature for 1 h. To this reaction mixture, borontriflouride-etherate (14.76 mL, 117.35 mmol) was added slowly at 0° C. and the reaction mixture was maintained at room temperature for 3 h. After completion of the reaction, the reaction mixture was quenched with ice-water. Methanol (2 mL) was added and heated to 80° C. for 10 h. The solvent was distilled off, diluted with water, basified with 6 N NaOH and the product was extracted with ethyl acetate (2×50 mL). The combined organic layer was washed with brine solution, dried over anhydrous sodium sulphate and concentrated under reduced pressure to afford 1.7 g of (1R,5S,6s)-3-methyl-6-nitro-3-azabicyclo[3.1.0]hexane (Intermediate 109) as colorless liquid in a 58% yield.

¹H NMR (400 MHz, CDCl₃): δ 2.28 (s, 3H), 2.42 (m, 4H), 3.13 (m, 2H), 4.55 (s, 1H).

MASS (APCI−ve Scan) m/z 143 (M+H)

### Intermediate 110

(1R,5S,6s)-3-methyl-3-azabicyclo[3.1.0]hexan-6-amine

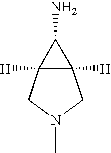

To a stirred solution of (1R,5S,6s)-3-methyl-6-nitro-3-azabicyclo[3.1.0]hexane (Intermediate 109) (1.7 g, 11.97 mmol) in methanol was added palladium on carbon (1.0 g) under nitrogen atmosphere in Parr hydrogenator vessel. The reaction mixture was maintained at room temperature under H₂ (50 psi) for 3 h. After completion of the reaction, the reaction mixture was filtered through a bed of Celite and washed with methanol. The filtrate was concentrated under reduced pressure to afford 900 mg of (1R,5S,6s)-3-methyl-3-azabicyclo[3.1.0]hexan-6-amine (Intermediate 110) as oily compound in a 67% yield.

¹H NMR (400 MHz, CDCl₃): δ 2.26 (s, 3H), 2.34 (m, 3H), 2.57 (m, 1H), 2.85 (m, 1H), 3.01 (m, 1H), 3.47 (s, 1H).

MASS (APCI+ve Scan) m/z 113 (M+H)

### Intermediate 111

(R)-ethyl 1-((1-(tert-butoxycarbonyl)pyrrolidin-3-yl)methyl)-6-iodo-4-oxo-1,4-dihydroquinoline-3-carboxylate:

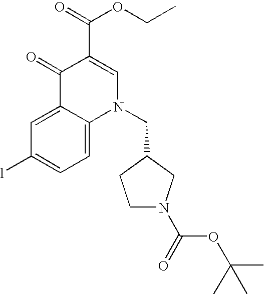

(R)-3-(Aminomethyl)-1-N-Boc-pyrrolidine (994 mg, 4.86 mmol) was added to the stock solution of (Z)-ethyl 3-(dimethylamino)-2-(3-iodobenzoyl)acrylate (Intermediate 11) (0.34M, 13 mL, 4.42 mmol). The mixture was stirred at room temperature overnight. The solvent was removed in vacuo and the residue was dried in a vacuum oven for 1.5 hours. The residue was then dissolved in dimethyl formamide (8 mL) and potassium carbonate powder (670 mg, 4.86 mmol) was added. The reaction was heated at 60° C. for overnight then cooled to room temperature. Water (50 mL) was added and the suspension was extracted with ethyl acetate (3×, 50 mL). The organic layers were dried over sodium sulfate, and the solvent was removed in vacuo to give quantitative (R)-ethyl 141-(tert-butoxycarbonyl)pyrrolidin-3-yl)methyl)-6-iodo-4-oxo-1,4-dihydroquinoline-3-carboxylate as a brown sticky solid.

MS (ESP): 527.1 (M+H⁺) for C₂₂H₂₇IN₂O₅

¹H NMR (300 MHz, DMSO-d₆): δ ppm 1.42 (t, 3H), 1.46 (s, 9H), 1.71 (m, 1H), 2.02 (bm, 1H), 2.78 (bs, 1H), 3.12 (bs, 1H), 3.25-3.62 (bm, 3H), 3.96-4.40 (bm, 2H), 4.42 (q, 2H), 7.19 (d, 1H), 7.92 (dd, 1H), 8.40 (s, 1H), 8.83 (s, 1H)

### Intermediate 112

The following Intermediates were prepared according to the procedure described for Intermediate 111 from the starting material listed in the table.

| Int | Compound | Data | SM |
|---|---|---|---|
| 112 | (S)-ethyl 1-((1-(tert- butoxycarbonyl)pyrrolidin-3- yl)methyl)-6-iodo-4-oxo-1,4- dihydroquinoline-3-carboxylate 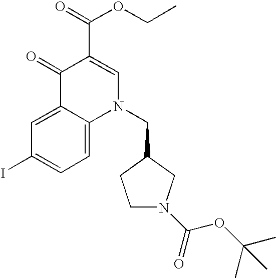 | MS (ESP): 527.1 (M + H⁺) for C₂₂H₂₇IN₂O₅ ¹H NMR (300 MHz, DMSO- d₆): δ ppm l.42 (t, 3H), 1.46 (s, 9H), 1.71 (m, 1H), 2.02 (bm, 1H), 2.78 (bs, 1H), 3.12 (bs, 1H), 3.25-3.62 (bm, 3H), 3.96- 4.40 (bm, 2H), 4.42 (q, 2H), 7.19 (d, 1H), 7.92 (dd, 1H), 8.40 (s, 1H), 8.83 (s, 1H) | Intermediate 11 and (S)-3- (Aminomethyl)- 1-N-Boc- pyrrolidine |

### Intermediate 113

### (R)-ethyl 6-iodo-4-oxo-1-(pyrrolidin-3-ylmethyl)-1,4-dihydroquinoline-3-carboxylate hydrochloride

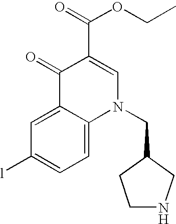

The crude (R)-ethyl 1-((1-(tert-butoxycarbonyl)pyrrolidin-3-yl)methyl)-6-iodo-4-oxo-1,4-dihydroquinoline-3-carboxylate (Intermediate 111) (˜4.4 mmol) was dissolved in 1,4-dioxane (20 mL) and 4.0M hydrogen chloride in 1,4-dioxane (20 mL) was added. The reaction was stirred at room temperature for 1 hour, and the solvent was removed in vacuo. The solid was then dried in a vacuum oven at 40° C. for 18 hours to give 2.3 g (quant. yield) of (R)-ethyl 6-iodo-4-oxo-1-(pyrrolidin-3-ylmethyl)-1,4-dihydroquinoline-3-carboxylate hydrochloride as a yellow solid.

MS (ESP): 427.1 (M+H⁺) for C₁₇H₁₉IN₂O₃

### Intermediate 114

The following Intermediates were prepared according to the procedure described for Intermediate 113 from the starting material listed in the table.

| Int | Compound | Data | SM |
|---|---|---|---|
| 114 | (S)-ethyl 6-iodo-4-oxo-1-(pyrrolidin- 3-ylmethyl)-1,4-dihydroquinoline-3- carboxylate hydrochloride. 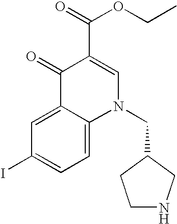 | MS (ESP): 427.1 (M + H⁺) for C₁₇H₁₉IN₂O₃ | Intermediate 112 and HCl |

### Intermediate 115

(R)-ethyl 6-(6-(3-ethylureido)-4-(4-(trifluoromethyl)thiazol-2-yl)pyridin-3-yl)-4-oxo-1-(pyrrolidin-3-ylmethyl)-1,4-dihydroquinoline-3-carboxylate.

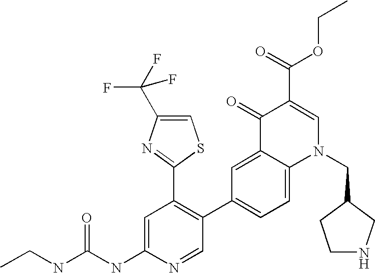

To a tall vial was charged (R)-ethyl 6-iodo-4-oxo-1-(pyrrolidin-3-ylmethyl)-1,4-dihydroquinoline-3-carboxylate hydrochloride (Intermediate 113) (1.0 g, 2.16 mmol), 6-(3-ethylureido)-4-(4-trifluoromethylthiazol-2-yl)pyridin-3-ylboronic acid (Intermediate 12) (940 mg, 2.61 mmol) and sodium bicarbonate (730 mg, 8.65 mmol) in dimethoxyethane (10 mL) and water (2 mL). The reaction mixture was purged with N₂ for about 5 minutes, then trans dichlorobis(triphenylphosphine)palladium (II) (160 mg, 0.228 mmol) was added. The reaction was heated for 3 hours at 80-85° C. The reaction went to completion based on LC. The mixture was cooled to room temperature and water (10 mL) was added. The precipitate was collected to give fairly clean (R)-ethyl 6-(6-(3-ethylureido)-4-(4-(trifluoromethyl)thiazol-2-yl)pyridin-3-yl)-4-oxo-1-(pyrrolidin-3-ylmethyl)-1,4-dihydroquinoline-3-carboxylate (1.1 g, 82.7%) as a light brown solid after drying under vacuum at 40° C. overnight.

MS (ESP): 615.3 (M+H⁺) for C₂₉H₂₉F₃N₆O₄S

### Intermediate 116

The following Intermediates were prepared according to the procedure described for Intermediate 115 from the starting material listed in the table.

| Int | Compound | Data | SM |
|---|---|---|---|
| 116 | (S)-ethyl 6-(6-(3-ethylureido)-4-(4- (trifluoromethyl)thiazol-2-yl)pyridin- 3-yl)-4-oxo-1-(pyrrolidin-3-ylmethyl)- 1,4-dihydroquinoline-3-carboxylate. 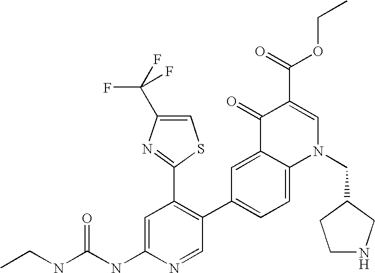 | MS (ESP): 615.3 (M + H⁺) for C₂₉H₂₉F₃N₆O₄S | Intermediate 114 and 12 |

### Intermediate 117

### (R)-ethyl 1-((1-(2-(4-(tert-butoxycarbonyl)piperazin-1-yl)ethyl)pyrrolidin-3-yl)methyl)-6-(6-(3-ethylureido)-4-(4-(trifluoromethyl)thiazol-2-yl)pyridin-3-yl)-4-oxo-1,4-dihydroquinoline-3-carboxylate

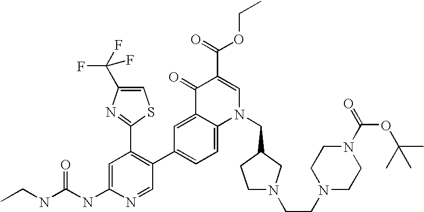

To a vial was charged (R)-ethyl 6-(6-(3-ethylureido)-4-(4-(trifluoromethyl)thiazol-2-yl)pyridin-3-yl)-4-oxo-1-(pyrrolidin-3-ylmethyl)-1,4-dihydroquinoline-3-carboxylate (Intermediate 115) (50 mg, 0.081 mmol), tert-butyl 4-(2-chloroethyl)piperazine-1-carboxylate (23 mg, 0.090 mmol) and potassium carbonate (15 mg, 0.11 mmol) in anhydrous acetonitrile (2 mL) and the resulting mixture was stirred at 60-65° C. for 1.5 hours. The mixture was then diluted with saturated sodium bicarbonate and the product extracted with ethyl acetate. The organics were dried over sodium sulfate, concentrated, and purified with another lot (˜1.3 mmol) by Analogix eluting with dichloromethane/methanol.

MS (ESP): 827.1 (MH⁺) for C₄₀H₄₉F₃N₈O₆S

### Intermediate 118

The following Intermediates were prepared according to the procedure described for Intermediate 117 from the starting material listed in the table.

| Int | Compound | Data | SM |
|---|---|---|---|
| 118 | (S)-ethyl 1-((1-(2-(4-(tert- butoxycarbonyl)piperazin-1- yl)ethyl)pyrrolidin-3-yl)methyl)-6-(6- (3-ethylureido)-4-(4- (trifluoromcthyl)thiazol-2-yl)pyridin- 3-yl)-4-oxo-1,4-dihydroquinoline-3- carboxylate. 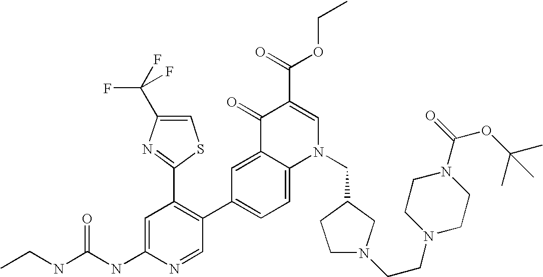 | MS (ESP): 827.1 (MH⁺) for C₄₀H₄₉F₃N₈O₆S | Intermediate 116 and tert- butyl 4-(2- chloroethyl) piperazine-1- carboxylate |

### Intermediate 119

### (R)-ethyl 6-(6-(3-ethylureido)-4-(4-(trifluoromethyl)thiazol-2-yl)pyridin-3-yl)-4-oxo-1-((1-(2-(piperazin-1-yl)ethyl)pyrrolidin-3-yl)methyl)-1,4-dihydroquinoline-3-carboxylate hydrochloride

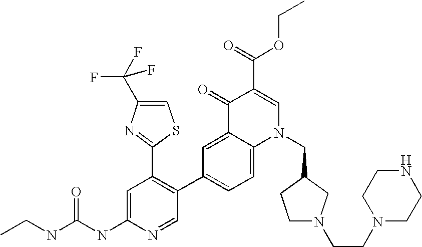

(R)-Ethyl 1-((1-(2-(4-(tert-butoxycarbonyl)piperazin-1-yl)ethyl)pyrrolidin-3-yl)methyl)-6-(6-(3-ethylureido)-4-(4-(trifluoromethyl)thiazol-2-yl)pyridin-3-yl)-4-oxo-1,4-dihydroquinoline-3-carboxylate (Intermediate 117) (200 mg) was dissolved in anhydrous 1,4-dioxane (20 mL) and 4M HCl in 1,4-dioxane (10 mL) was added. The solution was stirred at room temperature for 1-2 hours until complete and was concentrated to dryness to give a brown solid. The material was used ‘as is’ in the next reaction.

MS (ESP): 727.2 (MH⁺) for free base C₃₅H₄₂ClF₃N₈O₄S

### Intermediate 120

The following Intermediates were prepared according to the procedure described for Intermediate 119 from the starting material listed in the table.

| Int | Compound | Data | SM |
|---|---|---|---|
| 120 | (S)-ethyl 6-(6-(3-ethylureido)-4-(4- (trifluoromethyl)thiazol-2-yl)pyridin- 3-yl)-4-oxo-1-((1-(2-(piperazin-1- yl)ethyl)pyrrolidin-3-yl)methyl)-1,4- dihydroquinoline-3-carboxylate hydrochloride. 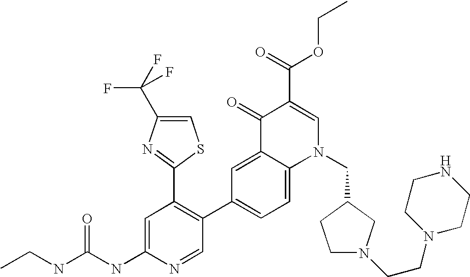 | MS (ESP): 727.2 (MH⁺) for free base C₃₅H₄₂ClF₃N₈O₄S | Intermediate 118 and HCl |

### Intermediate 121

### (R)-ethyl 6-(6-(3-ethylureido)-4-(4-(trifluoromethyl)thiazol-2-yl)pyridin-3-yl)-1-((1-(2-(4-methylpiperazin-1-yl)ethyl)pyrrolidin-3-yl)methyl)-4-oxo-1,4-dihydroquinoline-3-carboxylate

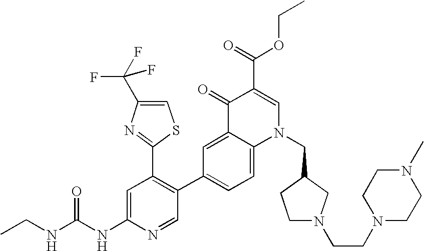

To a vial was charged (R)-ethyl 6-(6-(3-ethylureido)-4-(4-(trifluoromethyl)thiazol-2-yl)pyridin-3-yl)-4-oxo-1-((1-(2-(piperazin-1-yl)ethyl)pyrrolidin-3-yl)methyl)-1,4-dihydroquinoline-3-carboxylate hydrochloride (Intermediate 119) (350 mg, 0.459 mmol) in methanol (20 mL) and MP-cyanoborohydride (293 mg, 0.69 mmol) beads were added followed by formaldehyde (37% in water, 0.3 mL) and the resulting mixture was allowed to stir in a warm water bath for 2 hours. Reaction was incomplete so additional MP-cyanoborohydride and formaldehyde was added and stirring continued at room temperature overnight. The mixture was filtered and the beads were washed with more methanol. The filtrate was concentrated and used in the next step without further purification.

MS (ESP): 741.3 (MH⁺) for C₃₆H₄₃F₃N₈O₄S

### Intermediate 122

The following Intermediates were prepared according to the procedure described for Intermediate 121 from the starting material listed in the table.

| Int | Compound | Data | SM |
|---|---|---|---|
| 122 | (S)-ethyl 6-(6-(3-ethylureido)-4-(4- (trifluoromethyl)thiazol-2-yl)pyridin- 3-yl)-1-((1-(2-(4-methylpiperazin-1- yl)ethyl)pyrrolidin-3-yl)methyl)-4- oxo-1,4-dihydroquinoline-3- carboxylate. 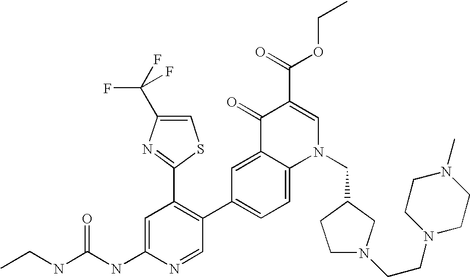 | MS (ESP): 741.0 (MH⁺) for C₃₆H₄₃F₃N₈O₄S | Intermediate 120 |

### Intermediates 123-124

The following intermediates were prepared according to the procedure described for Intermediate 81 from the starting materials listed in the table.

| Int | Compound | Data | SM |
|---|---|---|---|
| 123 | (R)-7-(2-(diethylamino)ethoxy)-1- ((1-ethylpyrrolidin-2-yl)methyl)-6- iodo-4-oxo-1,4-dihydroquinoline-3- carboxylic acid 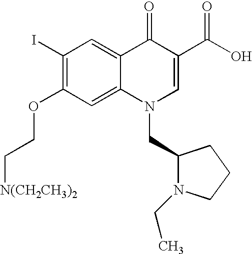 | Calcd for C₂₃H₃₂IN₃O₄ [M + H]⁺: 542.12. H¹NMR (d₆-DMSO) δ 15.14 (s, 1H), 8.87 (s, 1H), 8.68 (s, 1H), 7.36 (s, 1H), 4.64 (m, 1H), 4.38-4.3 (m, 2H), 3.09-3.06 (m, 2H), 2.93- 2.90 (m, 2H), 2.65-2.6 (m, 4H), 2.26-2.12 (m, 4H), 1.73-1.68 (m, 2H), 1.02 (t, 6H), 0.73 (t, 3H). | Intermediate 125 & 2- (diethylamino) ethanol |
| 124 | 7-(2-(dimethylamino)ethoxy)-1- ethyl-6-iodo-4-oxo-1,4- dihydroquinoline-3-carboxylic acid 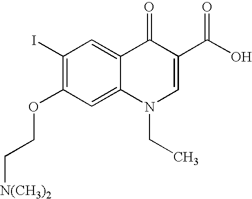 | Calcd for C₁₆H₁₉IN₂O₄ [M + H]⁺: 431. H¹NMR (d₆-DMSO) δ 15.16 (s, 1H),9.0 (s, 1H), 8.69 (s, 1H), 7.28 (s, 1H), 4.64-4.57 (m, 2H), 4.44- 4.41 (m, 2H), 2.88-2.85 (m, 2H), 2.37 (s, 6H), 1.42 (t, 3H | Intermediate 78 & 2- (dimethylamino) ethanol |

### Intermediates 125

The following intermediate was prepared according to the procedure described for Intermediate 78 from the starting materials listed in the table.

| Int | Compound | Data | SM |
|---|---|---|---|
| 125 | (R)-1-((1-ethylpyrrolidin-2- yl)methyl)-7-fluoro-6-iodo-4-oxo- 1,4-dihydroquinoline-3-carboxylic acid 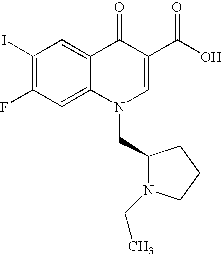 | Calcd for C₁₇H₁₈IN₂O₃ [M + H]⁺: 445.04. H¹NMR (d₆-DMSO) δ 14.78 (s, 1H), 8.93 (s, 1H), 8.76 (s, 1H), 8.15 (s, 1H), 4.58-4.56 (m, 1H), 3.1-2.93 (m, 2H), 2.27-1.87 (m, 4H), 1.7-1.68 (m, 2H), 0.7 (t, 3H). | Intermediate 126 |

### Intermediates 126

The following intermediate was prepared according to the procedure described for Intermediate 10 from the starting materials listed in the table.

| Int | Compound | Data | SM |
|---|---|---|---|
| 126 | (R)-ethyl 1-((1-ethylpyrrolidin-2- yl)methyl)-7-fluoro-6-iodo-4-oxo- 1,4-dihydroquinoline-3-carboxylate 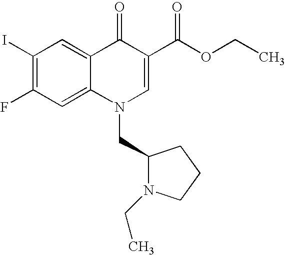 | Calcd for C₁₉H₂₂IN₂O₃ [M + H]⁺: 473.09. | Intermediate 83 & (R)-(1- ethylpyrrolidin- 2-yl) methanamine |

### Intermediates 127

The following intermediates were prepared according to the procedure described for Intermediate 81 from the starting materials listed in the table.

| Int | Compound | Data | SM |
|---|---|---|---|
| 127 | 1-(2-(dimethylamino)ethyl)-6- iodo-7-(2-morpholinoethoxy)-4- oxo-1,4-dihydroquinoline-3- carboxylic acid 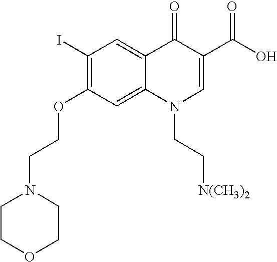 | Calcd for C₂₀H₂₆IN₃O₅ [M + H]⁺: 516. H¹NMR (d₆-DMSO) δ 15.15 (s, 1H), 8.86 (s, 1H), 8.69 (s, 1H), 7.29 (s, 1H), 4.68-4.64 (m, 2H), 4.44-4.4 (m, 2H), 3.61-3.58 (m, 4H), 2.87-2.83 (m, 2H), 2.65-2.62 (m, 2H), 2.59-2.56 (m, 4H), 2.2 (s, 6H). | Intermediate 80 & 2- morpholinoethanol |

### Intermediates 128-130

The following intermediates were prepared according to the procedure described for Intermediate 10 from the starting materials listed in the table.

| Int | Compound | Data | SM |
|---|---|---|---|
| 128 | ethyl 7-fluoro-6-iodo-1-(2- morpholinoethyl)-4-oxo-1,4- dihydroquinoline-3-carboxylat0e 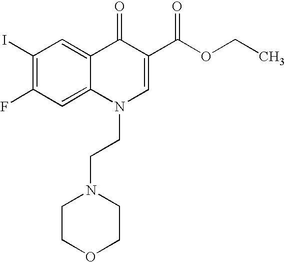 | Calcd for C₂₀H₂₆IN₃O₅ [M + H]⁺: Calcd for C₁₈H₂₀FIN₂O₄ [M + H]⁺: 475.07. H¹NMR (d₆-DMSO) δ 8.74 (s, 1H), 8.61 (s, 1H), 7.93 (s, 1H), 4.79-4.71 (m, 2H), 4.28-4.21 (m, 2H), 4.05-3.97 (m, 2H), 3.77-3.68 (m, 2H), 3.6-3.47 (m, 4H), 3.2-3.09 (m, 2H), 1.28 (t, 3H). | Intermediate 83 & 2morpholino- ethanamine |
| 129 | ethyl 1-(2-(dimethylamino)propyl)- 7-fluoro-6-iodo-4-oxo-1,4- dihydroquinoline-3-carboxylate 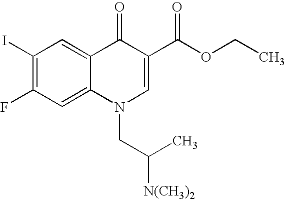 | Calcd for C₁₇H₂₀FIN₂O₃ [M + H]⁺: 468.94. H¹NMR (d₆-DMSO) δ 8.6 (d, 1H), 8.51 (s, 1H), 7.84 (d, 1H), 4.33- 4.14 (m, 2H), 2.99-2.9 (m, 1H), 2.12 (s, 6H), 1.27 (t, 3H), 0.96 (d, 3H). | Intermediate 83 & N2,N2- dimethylpropane- 1,2-diamine |
| 130 | (S)-ethyl 7-fluoro-1-(1-hydroxy-4- methylpentan-2-yl)-6-iodo-4-oxo- 1,4-dihydroquinoline-3-carboxylate 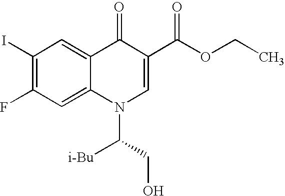 | Calcd for C₁₈H₂₁FINO₄ [M + H]⁺: 462.0. H¹NMR (d₆-DMSO) δ 8.63 (d, 1H), 8.58 (s, 1H), 8.09 (d, 1H), 5.13-5.1 (m, 1H), 4.27-4.2 (m, 2H), 3.71-3.68 (m, 2H), 1.89-1.84 (m, 1H), 1.75-1.68 (m, 1H), 1.48-1.36 (m, 1H), 1.27 (t, 3H), 0.9 (d, 3H), 0.85 (d, 3H). | Intermediate 83 & (S)- Leucinol |

### Intermediate 131

### 6-(4-(4-((R)-1-(benzyloxycarbonylamino)-2-methylpropyl)thiazol-2-yl)-6-(3-ethylureido)pyridin-3-yl)-1-((S)-1-hydroxy-3,3-dimethylbutan-2-yl)-4-oxo-1,4-dihydroquinoline-3-carboxylic acid

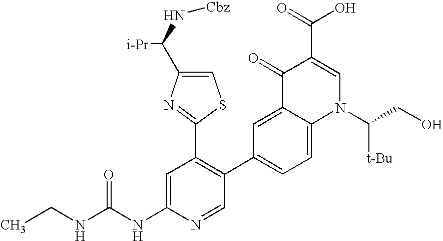

To a suspension of (S)-ethyl 1-(1-hydroxy-4-methylpentan-2-yl)-4-oxo-6-(4,4,5,5-tetramethyl-1,3,2-dioxaborolan-2-yl)-1,4-dihydroquinoline-3-carboxylate (230 mg, 0.52 mmol, 1 equiv.) (Intermediate 132) and (R)-benzyl 1-(2-(5-bromo-2-(3-ethylureido)pyridin-4-yl)thiazol-4-yl)-2-methylpropylcarbamate (Intermediate 136) (279 mg, 0.52 mmol, 1 equiv.) in 1,4-dioxane (2 mL) was added tetrakis(triphenylphosphine)palladium(0) (59.9 mg, 0.05 mmol, 0.1 equiv.) followed by a solution of cesium carbonate (338 mg, 1.04 mmol, 2 equiv.) in water (0.67 mL). The reaction was stirred at 100° C. for 4 h. 2N lithium hydroxide (0.52 mL) was added and stirred at 100° C. for 9 h. The reaction was cooled to 23° C. and diluted with water. 1 N HCl was added until pH 3-4 was reached. The precipitate was washed with water and hexanes and dried. The compound was purified via silica gel chromatography and concentrated to provide 6-(4-(4-((R)-1-(benzyloxycarbonylamino)-2-methylpropyl)thiazol-2-yl)-6-(3-ethylureido)pyridin-3-yl)-1-((S)-1-hydroxy-3,3-dimethylbutan-2-yl)-4-oxo-1,4-dihydroquinoline-3-carboxylic acid (461 mg, >99%).

Calcd for C₄₁H₄₈N₆O₇S [M+H]⁺: 741.21.

H¹ NMR (d₆-DMSO) δ 15.06 (s, 1H), 9.42 (s, 1H), 8.87 (s, 1H), 8.4 (s, 1H), 8.32 (s, 1H), 8.18 (s, 1H), 8.04 (s, 1H), 7.7 (t, 1H), 7.65-7.52 (m, 3H), 7.44-7.3 (m, 3H), 5.11-5.07 (m, 3H), 5.01-4.95 (m, 2H), 4.45 (m, 1H), 4.07 (m, 2H), 3.26-3.17 (m, 2H), 1.11 (t, 3H), 1.1-1.7 (m, 2H), 0.98 (s, 9H), 0.56 (m, 6H).

### Intermediate 132

### (S)-ethyl 1-(1-hydroxy-3,3-dimethylbutan-2-yl)-4-oxo-6-(4,4,5,5-tetramethyl-1,3,2-dioxaborolan-2-yl)-1,4-dihydroquinoline-3-carboxylate

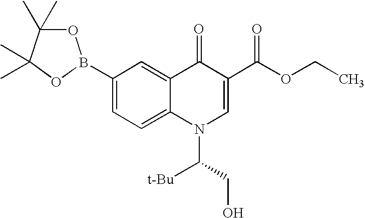

To a solution of (S)-ethyl 1-(1-hydroxy-3,3-dimethylbutan-2-yl)-6-iodo-4-oxo-1,4-dihydroquinoline-3-carboxylate (1 g, 2.26 mmol, 1 equiv.) (Intermediate 10) in 1,4-dioxane (10 mL) was added bis(triphenylphosphine)palladium chloride (0.158 g, 0.23 mmol, 0.1 equiv.). The solution was warmed up to 100° C. and 4,4,4′,4′,5,5,5′,5′-octamethyl-2,2′-bi(1,3,2-dioxaborolane) (1.719 g, 6.77 mmol, 3 equiv.) was added followed by triethylamine (0.943 mL, 6.77 mmol, 3 equiv.) and potassium acetate (0.664 g, 6.77 mmol, 3 equiv.). This was stirred at 100° C. for 8 h. The reaction was cooled to 23° C. and diluted with water. The precipitate was washed with water and hexanes and dried to afford (S)-ethyl 1-(1-hydroxy-3,3-dimethylbutan-2-yl)-4-oxo-6-(4,4,5,5-tetramethyl-1,3,2-dioxaborolan-2-yl)-1,4-dihydroquinoline-3-carboxylate (1 g, >99%). Calcd for C₂₄H₃₄BNO₆ [M+H]⁺: 444.08.

### Intermediate 133

### (Z)-Ethyl 3-(dimethylamino)-2-(2-fluoro-5-iodobenzoyl)acrylate

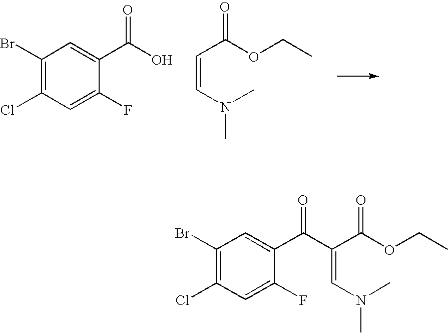

5-bromo-4-chloro-2-fluorobenzoic acid (5.00 g, 19.73 mmol, combi-blocks) was suspended in toluene and added thionyl chloride (7.2 mL, 98.64 mmol, Aldrich) and DMF (0.5 ml). Mixture was heated to reflux for 30 min. The reaction mixture was cooled to room temperature and concentrated under reduced pressure to a yellow residue. The residue was dissolved in toluene (30 mL) and concentrated under reduced pressure again. To a solution of the resulting residue in toluene (30 mL) was added triethylamine (4.10 mL, 29.59 mmol, Acros) and (Z)-ethyl 3-(dimethylamino)acrylate (3.67 mL, 25.65 mmol, Acros) and the reaction mixture was heated to 90° C. for 1 h. The reaction mixture was cooled to room temperature and diluted with water and extracted three times with ethyl acetate. The combined organic extracts were washed with 1NHCl, brine and water then dried over magnesium sulfate, filtered and concentrated under reduced pressure to give a dark oil. This oil was triturated with ether to give a tan solid that was filtered off and washed with ether. Recovered (Z)-Ethyl 3-(dimethylamino)-2-(2-fluoro-5-iodobenzoyl)acrylate (6.90 g, 92%).

MS (ES) (M+H)⁺: 379 for C₁₄H₁₄BrClFNO₃

### Intermediates 134

The following Intermediate was prepared by the procedure described in Intermediate 10 from the starting materials indicated.

| Int | Compound | Data | SM |
|---|---|---|---|
| 134 | ethyl 6-bromo-7-chloro-1- cyclopropyl-4-oxo-1,4- dihydroquinoline-3- carboxylate 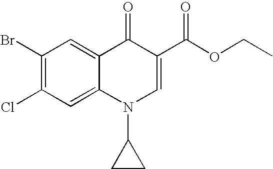 | MS (ES) (M + H)⁺: 371 for C₁₅H₁₃BrClNO₃ | Intermediate 133 and cyclo- propanamine |

### Intermediate 135

The following Intermediates were prepared according to the procedure described for Intermediate 9 from the starting materials indicated.

| Int | Compound | Data | SM |
|---|---|---|---|
| 135 | ethyl 7-chloro-1-cyclopropyl-6-(6-(3- ethylureido)-4-(4- (trifluoromethyl)thiazol-2-yl)pyridin- 3-yl)-4-oxo-1,4-dihydroquinoline-3- carboxylate 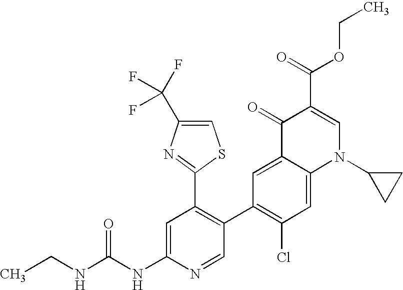 | MS (ES) (M + H)⁺: 607 for C₂₇H₂₃ClF₃N₅O₄S | Intermediate 134 and 12 |

### Intermediate 136

### Benzyl [(1R)-1-(2-{5-bromo-2-[(ethylcarbamoyl)amino]pyridin-4-yl}-1,3-thiazol-4-yl)-2-methylpropyl]carbamate

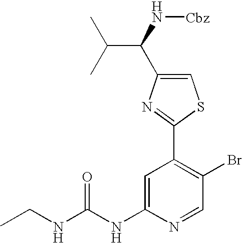

To a stirred solution of 5-bromo-2-[(ethylcarbamoyl)amino]pyridine-4-carbothioamide (Intermediate 15) (5.54 g, 18.34 mmol) in acetonitrile (600 mL) and ethanol (300 mL) was added benzyl [(3R)-1-bromo-4-methyl-2-oxopentan-3-yl]carbamate (Intermediate 137) (12 g, 36.69 mmol) and heated to reflux for 48 h. After completion of the reaction, the reaction mixture was cooled to room temperature and concentrated under reduced pressure. The crude product was purified by column chromatography by over neutral alumina to afford 5.5 g (56.4%) of benzyl [(1R)-1-(2-{5-bromo-2-[(ethylcarbamoyl)amino]pyridin-4-yl}-1,3-thiazol-4-yl)-2-methylpropyl]carbamate.

¹H NMR (400 MHz, DMSO d₆): δ 0.872 (m, 6H), 1.04 (t, 3H), 2.23 (m, 1H), 3.15 (q, 2H), 4.70 (t, 1H), 5.06 (d, 2H), 7.33 (m, 6H), 7.78 (m, 2H), 8.32 (s, 1H), 8.50 (s, 1H), 9.32 (s, 1H).

LC-MS: m/z 534.3 (M+2)

### Intermediate 137

### Benzyl [(3R)-1-bromo-4-methyl-2-oxopentan-3-yl]carbamate

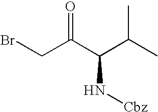

To a stirred solution of Benzyl [(3R)-1-diazo-4-methyl-2-oxopentan-3-yl]carbamate (Intermediate 138) (16 g, 58.18 mmol) in ether (1 L) was added 33% hydrogen bromide in acetic acid (7.97 g, 99.26 mmol), slowly at 0° C. and allowed to stir for 30 min at 0° C. The progress of the reaction was monitored by TLC. After completion of the reaction, the reaction mixture was diluted with water (500 mL) and the product was extracted with ether (4×50 mL). The combined organic layer was washed with saturated sodium bicarbonate solution, brine solution, dried over anhydrous sodium sulphate and concentrated under reduced pressure to afford 12 g Benzyl [(3R)-1-bromo-4-methyl-2-oxopentan-3-yl]carbamate.

### Intermediate 138

### Benzyl[(3R)-1-diazo-4-methyl-2-oxopentan-3-yl]carbamate

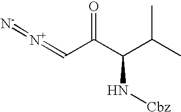

To a stirred solution of N-[(benzyloxy)carbonyl]-D-valine (Intermediate 139) (2 g, 7.96 mmol) in diethyl ether and tetrahydrofuran (20+20 mL) cooled to −20° C., triethylamine (0.80 g, 7.92 mmol) was slowly added followed by the addition of isobutyl chloroformate (1.08 g, 7.96 mmol) and stirred for 30 min at −20° C. The temperature was slowly raised to −10° C. and stirred for 20 min The progress of the reaction was monitored by TLC. After completion of the reaction, the (2-methylpropoxy)carbonyl N-[(benzyloxy)carbonyl]-D-valinate was slowly cannulated to freshly prepared diazomethane (1.3-1.6 g, 30.95-38.09 mmol) in ether at −60-−50° C. The temperature was slowly raised to room temperature and stirred for 2 h. The progress of the reaction was monitored by TLC. After completion of the reaction, the reaction mixture was washed with saturated sodium bicarbonate solution (100 mL), the aqueous phase was extracted with ether (2×50 mL). The combined organic layer was washed with brine solution, dried over anhydrous sodium sulphate and concentrated to afford 1.7 g, (77.6%) of benzyl [(3R)-1-diazo-4-methyl-2-oxopentan-3-yl]carbamate.

¹H NMR (400 MHz, CDCl₃): δ 0.90-0.98 (m, 6H), 2.03 (m, 1H), 4.05 (m, 2H), 5.10 (m, 2H), 5.39 (brs, 1H), 7.35 (m, 5H).

### Intermediate 139

### N-[(benzyloxy)carbonyl]-D-valine

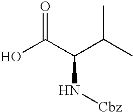

To a stirred solution of D-valine (600 mg, 5.12 mmol) in 1 N sodium hydroxide solution (205 mg, 5.12 mmol) benzyl chloroformate (1.65 g, 9.74 mmol) was added slowly in a dropwise manner at 0° C., (while adding benzyl chloroformate the pH was maintained at 8-9). The temperature of the reaction mixture was raised to room temperature and stirred for 2 h. The progress of the reaction was monitored by TLC. After completion of the reaction, the reaction mixture was washed with diethyl ether (20 mL), the organic layer was separated, the aqueous layer was cooled to 5-10° C. and the pH was adjusted to 1-2 with concentrated hydrochloric acid. The aqueous layer was extracted with ethyl acetate (2×50 mL), the combined organic layer was washed with brine solution, dried over anhydrous sodium sulphate and concentrated to afford 400 mg (33.3%) of N-[(benzyloxy) carbonyl]-D-valine.

¹H NMR (400 MHz, CDCl₃): δ 0.94 (d, 3H), 1.02 (d, 3H), 4.71 (m, 1H), 5.12 (s, 2H), 5.22 (d, 1H), 7.36 (m, 5H).

MASS (APCI−ve Scan): m/z 250.1 (M−H)

### Intermediate 140

### 1-(2-(dimethylamino)ethyl)-7-(2-(dimethylamino)ethylamino)-6-iodo-4-oxo-1,4-dihydroquinoline-3-carboxylic acid

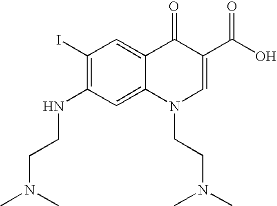

1-(2-(dimethylamino)ethyl)-7-fluoro-6-iodo-4-oxo-1,4-dihydroquinoline-3-carboxylic acid (Intermediate 80) (500 mg, 1.24 mmol) was dissolved in DMSO (2 mL). To the solution was added N1,N1-dimethylethane-1,2-diamine (0.162 mL, 1.48 mmol) and potassium carbonate (180 mg, 1.30 mmol). The solution was heated to 100° C. and monitored by LC/MS. Reaction is complete in about 2 hours, was cooled to room temperature, and was acidified with 2N HCl (pH=4-5). Collect the solids and dry over P₂O₅. Isolation gave 320 mg of the title compound as a yellow solid having a ¹H NMR consistent with the desired reaction product.

LC/MS (ES⁺)[(M+H)⁺]: 473 for C₁₈H₂₅IN₄O₃. ¹H NMR (300 MHz, d₆-DMSO): 2.23 (s, 6H), 2.38 (s, 6H), 2.69 (t, 2H), 2.74 (m, 2H), 3.50 (m, 2H), 4.60 (t, 2H), 6.09 (t, 1H), 6.70 (s, 1H), 8.56 (s, 1H), 8.75 (s, 1H).

### Intermediate 141

### Ethyl 6-iodo-1-(2-morpholinopropyl)-4-oxo-1,4-dihydroquinoline-3-carboxylate, hydrochloride salt

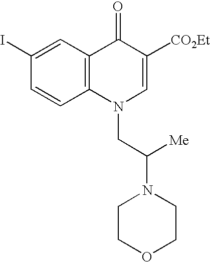

(Z)-Ethyl 3-(dimethylamino)-2-(2-fluoro-5-iodobenzoyl)acrylate (Intermediate 11) (0.500 g, 1.28 mmol) and 2-morpholinopropan-1-amine (0.184 g, 1.28 mmol) were diluted with THF (8 mL) and heated to 60° C. overnight. Potassium carbonate (0.530 g, 3.83 mmol) and DMF (4 mL) were added and the mixture was then heated to 70° C. for 2 days. The mixture was cooled to 0° C. and acidified to approx. pH 4 with 1 N aq HCl. The resultant solid was collected, washed with water, and used without additional purification.

LC/MS (ES⁺)[(M+H)⁺]: 471 for C₁₉H₂₃IN₂O₄

¹H NMR (DMSO-d₆+D₂O): δ 8.61 (s, 1H); 8.51 (d, 1H); 8.07 (dd, 1H); 7.54 (d, 1H); 4.61 (dd, 1H); 4.39 (dd, 1H); 4.19 (m, 2H); 3.75 (br s, 4H); 3.55 (m, 1H); 3.15 (m, 2H); 2.97 (m, 2H); 1.23 (t, 3H); 1.11 (d, 3H).

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

1. A compound of formula (IA): 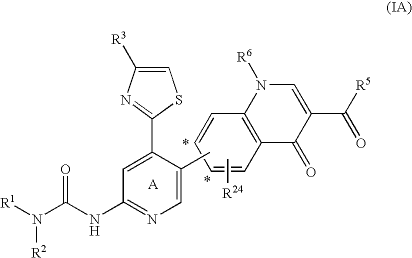
or a pharmaceutically acceptable salt thereof, wherein:
Ring A is attached to one of the carbon atoms indicated by “*”;
R¹ is selected from C₁₋₆alkyl, C₂₋₆alkenyl, C₂₋₆alkynyl or C₃₋₆cycloalkyl; wherein R¹ may be optionally substituted on carbon by one or more R⁷;
R² is selected from hydrogen or C₁₋₆alkyl; wherein said C₁₋₆alkyl may be optionally substituted by one or more groups independently selected from halo, cyano, hydroxy, nitro and amino;
or R¹ and R² together with the nitrogen to which they are attached form a heterocyclyl; wherein said heterocyclyl may be optionally substituted on one or more carbon atoms with one or more R⁸; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;
R³ is a C₁₋₆alkyl or a C₃₋₁₄carbocyclyl; wherein the alkyl or carbocyclyl may be optionally substituted on one or more carbon atoms by one or more R¹⁰;
R⁵ is —OH, a C₁₋₆alkoxy, an N—(C₁₋₆alkyl)amino, or N,N—(C₁₋₆alkyl)₂-amino; wherein the C₁₋₆alkoxy, an N—(C₁₋₆alkyl)amino, or N,N—(C₁₋₆alkyl)₂-amino may be optionally substituted on one or more carbon atoms with one or more, independently selected R¹⁴;
R⁶ is selected from the group consisting of hydrogen, C₁₋₁₀alkyl, C₃₋₁₄carbocyclyl-L-, and heterocycle-L-; wherein R⁶ is optionally substituted on one or more carbon atoms with one or more R¹⁶; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹⁷; provided that one of R⁵ or R⁶ is substituted with phosphonooxy, or R²⁴ is not H;
L is a direct bond or a C₁₋₆alkylene;
R⁷, R⁸, and R¹⁰ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆-carbocyclyl, and heterocyclyl;
wherein R⁷, R⁸, and R¹⁰, independently of each other may be optionally substituted on one or more carbon by one or more R¹⁹; and wherein if said heterocyclyl contains an —NH— moiety that nitrogen may be optionally substituted by a group selected from R²⁰; and wherein if said heterocyclyl contains an ═N— or a —S— moiety that nitrogen may be optionally substituted by one oxo group and that sulfur may be optionally substituted by one or two oxo groups;
R¹⁴ and R¹⁶ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆-carbocyclyl, heterocyclyl and phosphonooxy; wherein R¹⁴ and R¹⁶ independently of each other may be optionally substituted on one or more carbon by one or more R²¹; and wherein if said heterocyclyl contains an —NH— moiety that nitrogen may be optionally substituted by a group selected from R²²; and wherein if said heterocyclyl contains an ═N— or a —S— moiety that nitrogen may be optionally substituted by one oxo group and that sulfur may be optionally substituted by one or two oxo groups;
R⁹, R¹⁷, R²⁰, and R²², for each occurrence, are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹⁷, R²⁰, and R²², independently of each other, may be optionally substituted on carbon by one or more R²³; and
R¹⁹, R²¹, and R²³, for each occurrence, are independently selected from halo, nitro, cyano, hydroxy, trifluoromethoxy, trifluoromethyl, amino, carboxy, carbamoyl, mercapto, sulphamoyl, C1-6alkyl, C1-6alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
R²⁴ is selected from the group consisting of hydrogen, halo, nitro, cyano, hydroxy, amino, mercapto, heterocyclyl, C₁₋₆alkyl, C₂₋₆alkenyl, C₂₋₆alkynyl, C₁₋₆alkoxy, N—(C₁₋₆alkyl)amino, N,N—(C₁₋₆alkyl)₂-amino, and C₁₋₆alkylsulfanyl; wherein R²⁴ may be optionally substituted on one or more carbon by one or more one or more R²⁵; wherein if said heterocyclyl contains an —NH— moiety that nitrogen may be optionally substituted by C₁₋₆alkyl;
R²⁵ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆-carbocyclyl or heterocyclyl; wherein R²⁵ may be optionally substituted on one or more carbon by one or more R²⁶; and wherein if said heterocyclyl contains an —NH— moiety that nitrogen may be optionally substituted by a group selected from R²⁷; and wherein if said heterocyclyl contains an ═N— or a —S— moiety that nitrogen may be optionally substituted by one oxo group and that sulfur may be optionally substituted by one or two oxo groups;
R²⁶ and R²⁸, for each occurrence, are independently selected from halo, nitro, cyano, hydroxy, trifluoromethoxy, trifluoromethyl, amino, carboxy, carbamoyl, mercapto, sulphamoyl, C₁₋₆alkyl, C₁₋₆alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and
R²⁷, for each occurrence, is independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)₂-carbamoyl, benzyl, benzyloxycarbonyl, benzoyl and phenylsulphonyl;
wherein R²⁷ may be optionally substituted on carbon by one or more R²⁸.

2. The compound of claim 1, or a pharmaceutically acceptable salt thereof, wherein:
R¹ is a C₁₋₆alkyl;
R² is hydrogen;
R³ is a C₁₋₆alkyl or C₃₋₆carbocyclyl, wherein the alkyl or carbocycyl is optionally substituted with one or more halo;
R⁵ is —OH, a C₁₋₆alkoxy, or an N—(C₁₋₆alkyl)amino, wherein when R⁵ is an alkoxy or a N—(C₁₋₆alkyl)amino it is optionally substituted with phosphonooxy;
R⁶ is a C₁₋₆alkyl, a C₃₋₁₄carbocyclyl-L-, or a heterocyclyl-L-, wherein the alkyl, carbocyclyl-L and heterocyclyl are optionally substituted on one or more carbon atoms with one or more C₁₋₆alkly, C₁₋₆alkoxy, or phosphonooxy, provided that one of R⁵ or R⁶ is substituted with phosphonooxy.

3. The compound of claim 1 or 2, or a pharmaceutically acceptable salt thereof, wherein R¹ is a C₁₋₆alkyl.

4. The compound of claim 3, or a pharmaceutically acceptable salt thereof, wherein R¹ is ethyl.

5. The compound of any one of the preceding claims, or a pharmaceutically acceptable salt thereof, wherein R² is hydrogen.

6. The compound of any one of the preceding claims, or a pharmaceutically acceptable salt thereof, wherein R³ is trifluouromethyl or cyclopropyl.

7. The compound of any one of the preceding claims, or a pharmaceutically acceptable salt thereof, wherein R⁵ is selected from the group consisting of —OH, 2-phosphonooxy-ethylamino, and 3-phosphonooxy-propylamino.

8. The compound of any one of the preceding claims, or a pharmaceutically acceptable salt thereof, wherein R⁶ is a C₁₋₆alkyl which is substituted on one or more carbon atoms with one or more independently selected R¹⁶.

9. The compound of claim 8, or a pharmaceutically acceptable salt thereof, wherein R¹⁶, for each occurrence, is independently selected from hydroxy, a C₁₋₆alkoxy, a C₃₋₆carbocyclyl, a heterocyclyl, and phosphonooxy.

10. The compound of claim 9, or a pharmaceutically acceptable salt thereof, wherein R⁶ is ethyl, cyclopropylmethyl, isopentyl, propyl, 5-methyl-oxadiazol-3-ylmethyl, 1-phosphonooxy-4-methyl-pentan-2-yl, 1,3-dimethoxypropan-2-yl, 3,3-dimethylbutyl, 2-methoxyethyl, 1-phosphonooxy-butan-2-yl, 1-phosphonooxy-3,3-dimethyl-butan-2-yl, 1-phosphonooxy-3-methyl-butan-2-yl, or 1-methoxymethyl-2-methoxy-ethyl.

11. The compound of any one of claims 1-7, or a pharmaceutically acceptable salt thereof, wherein R⁶ is a C₃₋₁₄carbocyclyl-L- or a heterocycle-L-, wherein the carbocyclyl or the heterocyclyl is substituted on one or more carbon atoms with one or more independently selected R¹⁶.

12. The compound of claim 11, or a pharmaceutically acceptable salt thereof, wherein R⁶ is cyclopropylmethyl, 1-ethylpyrrolidin-2-yl)methyl, (1-methyl-1H-imidazol-4-yl)methyl, 2-morpholinopropyl, (2-(diethylamino)ethyl)piperidin-3-yl, cyclohexyl, 5-methyl-oxadiazol-3-ylmethyl, or cyclopropyl.

13. The compound of claim 1, or a pharmaceutically acceptable salts thereof, represented by the following formula:

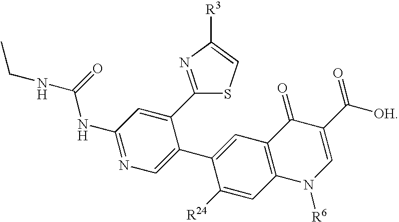

14. A pharmaceutical composition comprising a compound of any one of claims 1-13, or a pharmaceutically acceptable salt thereof, and a pharmaceutically acceptable excipient or carrier.

15. A method of inhibiting bacterial DNA gyrase and/or bacterial topoisomerase IV in a warm-blooded animal in need of such treatment, comprising administering to the animal an effective amount of a compound of any one of claims 1-13, or a pharmaceutically acceptable salt thereof.

16. A method of producing an antibacterial effect in a warm-blooded animal in need of such treatment, comprising administering to the animal an effective amount of a compound of any one of claims 1-13, or a pharmaceutically acceptable salt thereof.

17. A method of treating a bacterial infection in a warm-blooded animal in need thereof, comprising administering to the animal an effective amount of a compound of any one of claims 1-13, or a pharmaceutically acceptable salt thereof.

18. The method of claim 17, wherein the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

19. The method of any one of claims 15 through 18, wherein the warm-blooded animal is a human.

20. The use of a compound of any one of claims 1-13, or a pharmaceutically acceptable salt thereof, for the manufacture of a medicament for use in the production of an antibacterial effect in a warm-blooded animal.

21. The use of a compound of any one of claims 1-13, or a pharmaceutically acceptable salt thereof, for the manufacture of a medicament for use in inhibition of bacterial DNA gyrase and/or topoisomerase IV in a warm-blooded animal.

22. The use of a compound of any one of claims 1-13, or a pharmaceutically acceptable salt thereof, for the manufacture of a medicament for use the treatment of a bacterial infection in a warm-blooded animal.

23. The use of claim 22, wherein the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis and Vancomycin-Resistant Enterococci.

24. The use of any one of claims 20 through 23, wherein the warm-blooded animal is a human.

25. A compound of any one of claims 1-13, or a pharmaceutically acceptable salt thereof, for use in production of an anti-bacterial effect in a warm-blooded animal.

26. A compound of any one of claims 1-13, or a pharmaceutically acceptable salt thereof, for use in inhibition of bacterial DNA gyrase and/or topoisomerase IV in a warm-blooded animal.

27. A compound of any one of claims 1-13, or a pharmaceutically acceptable salt thereof, for use in the treatment of a bacterial infection in a warm-blooded animal.

28. A compound of any one of claims 1-13, or a pharmaceutically acceptable salt thereof, for use in the treatment of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis or Vancomycin-Resistant Enterococci.
